(19) United States
(12) Patent Application Publication
(10) Pub. No.: US 2024/0368233 A1
(43) Pub. Date: Nov. 7, 2024

(54) MODULAR RECONFIGURABLE ASYMMETRIC PROTEIN ASSEMBLIES

(71) Applicant: UNIVERSITY OF WASHINGTON, Seattle, WA (US)

(72) Inventors: David BAKER, Seattle, WA (US); Danny SAHTOE, Seattle, WA (US); Florian PRAETORIUS, Seattle, WA (US); Bart TIMMERMANS, Seattle, WA (US); Yang HSIA, Seattle, WA (US); Alexis COURBET, Seattle, WA (US); Natasha EDMAN, Seattle, WA (US)

(21) Appl. No.: 18/576,532

(22) PCT Filed: Jul. 11, 2022
(86) PCT No.: PCT/US2022/073589

§ 371 Date: Jan. 4, 2024

Related U.S. Application Data
(60) Provisional application No. 63/221,233, filed on Jul. 13, 2021.

(51) Int. Cl. C07K 14/435

(52) U.S. Cl. CPC C07K 14/435; C07K 2319/735

(57) ABSTRACT

Polypeptides and fusion proteins capable of heterodimer formation, methods for their use, and methods for their design are provided.

### CROSS REFERENCE

This application claims priority to U.S. Provisional Patent Application Ser. No. 63/221,233 filed Jul. 13, 2021, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Jul. 7, 2022 having the file name “21-0752-WO_SeqList.hml” and is 419 kb in size.

### BACKGROUND

Asymmetric multi-protein complexes that undergo subunit exchange play central roles in biology, but present a challenge for protein design. The individual components must contain interfaces enabling reversible addition to and dissociation from the complex, but be stable and well behaved in isolation. The design of reconfigurable asymmetric assemblies is a more difficult challenge, as there is no symmetry “bonus” favoring the target structure (as is attained for example in the closing of an icosahedral cage), and because the individual subunits must be stable and soluble proteins in isolation in order to reversibly associate or dissociate.

### SUMMARY

In one aspect, the disclosure provides polypeptides comprising an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-28, not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity. In one embodiment, 1, 2, 3, 4, 5, 6, 7, 8, 9, 10, 11, 12, 13, 14, 15, 16, 17, 18, 19, 20, 21, or all of identified interface amino acid residues are identical at that residue position to the reference polypeptide.

In another embodiment, the disclosure provides heterodimer-forming polypeptides, comprising the amino acid sequence selected from the group consisting of SEQ ID NOS: SEQ ID NOS:29-33, 35-55, and 190-191 or comprising the amino acid sequence of any one of SEQ ID NOS:29, 35-55, and 190-191, wherein any functional domains added fused to the polypeptides (whether N-terminal, C-terminal, or internal other than within the interface region), and wherein the 1, 2, 3, 4, or 5 N-terminal and/or C-terminal amino acid residues may be present or absent. In further embodiments, the disclosure provides heterodimer-forming polypeptides, comprising the amino acid sequence selected from the group consisting of SEQ ID NOS:56-77, or comprising the amino acid sequence of any one of SEQ ID NOS:56 and 60-77.

In another embodiment, the disclosure provides fusion proteins, comprising:
(a) the polypeptide of embodiment of the disclosure; and
(b) a second polypeptide; optionally including an amino acid linker between the polypeptide and the second polypeptide. In one embodiment, the second polypeptide comprises a repeat polypeptide. In another embodiment, the repeat protein comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:78-89.

In a further embodiment, the disclosure provides proteins, comprising an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 90-189 and 196-199, not including any functional domains added (whether N-terminal, C-terminal, or internal other than within the interface region), and wherein the 1, 2, 3, 4, or 5 N-terminal and/or C-terminal amino acid residues, as well as any N-terminal methionine residue, may be present or absent when considering the percent identity.

In other aspects, the disclosure provides nucleic acids encoding the polypeptide or fusion protein of any embodiment herein, expression vectors comprising the nucleic acid operatively linked to a suitable control sequence, and host cells comprising the nucleic acid or the expression vector.

In another embodiment, the disclosure provides heterodimers, comprising two polypeptides or fusion proteins according to embodiment herein, wherein the two polypeptides are capable of self-assembly to form a heterodimer. In one embodiment, the two polypeptides or fusion proteins are a Chain A and Chain B pair comprising an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of selected from the following pairs, not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity:
(a) one of SEQ ID NOS:1-5 and SEQ ID NO: 6;
(b) SEQ ID NO:7 and SEQ ID NO:8;
(c) SEQ ID NO:9 and SEQ ID NO: 10;
(d) SEQ ID NO:11 and SEQ ID NO: 12;
(e) SEQ ID NO:13 and SEQ ID NO: 14;
(f) SEQ ID NO:15 and SEQ ID NO: 16;
(g) SEQ ID NO:17 and SEQ ID NO: 18;
(h) SEQ ID NO:19 and SEQ ID NO:20;
(i) SEQ ID NO:21 and SEQ ID NO:22;
(j) SEQ ID NO:23 and SEQ ID NO:24;
(k) SEQ ID NO:25 and SEQ ID NO:26; and
(l) SEQ ID NO:27 and SEQ ID NO:28.

In another embodiment, the two polypeptides or fusion proteins are a Chain A and Chain B pair comprising the amino acid sequence selected from the following pairs:
(a) one of SEQ ID NOS:29-32 and SEQ ID NO:33;
(b) SEQ ID NO:190 and SEQ ID NO:191;
(c) SEQ ID NO:35 and SEQ ID NO:36;
(d) SEQ ID NO:37 and SEQ ID NO:38;
(e) SEQ ID NO:39 and SEQ ID NO:40;
(f) SEQ ID NO:41 and SEQ ID NO:42;
(g) SEQ ID NO:43 and SEQ ID NO:44;
(h) SEQ ID NO:46 and SEQ ID NO:47;
(i) SEQ ID NO:48 and SEQ ID NO:49;
(j) SEQ ID NO:50 and SEQ ID NO:51;
(k) SEQ ID NO:52 and SEQ ID NO:53;
(l) SEQ ID NO:54 and SEQ ID NO:55;
(m) one of SEQ ID NO:56-59 and SEQ ID NO:60;
(n) SEQ ID NO:61 and SEQ ID NO: 191;
(o) SEQ ID NO:62 and SEQ ID NO: 63;
(p) SEQ ID NO:64 and SEQ ID NO: 65;
(q) SEQ ID NO:66 and SEQ ID NO: 67;
(r) SEQ ID NO:68 and SEQ ID NO: 69;
(s) SEQ ID NO:70 and SEQ ID NO:71;
(t) SEQ ID NO:72 and SEQ ID NO:73;
(u) SEQ ID NO:74 and SEQ ID NO:75; and
(v) SEQ ID NO:76 and SEQ ID NO:77.

In another embodiment, the disclosure provides asymmetric hetero-oligomeric assemblies comprising a plurality (2, 3, 4, 5, 6, 7, 8, 9, 10, or more) of the heterodimers of any embodiment herein. In one embodiment, the assemblies comprise as provided in individual rows of Table 6 or 7, wherein each component comprises an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 90-189 and 196-199, or SEQ ID NOS: 90-189, not including any functional domains added (whether N-terminal, C-terminal, or internal other than within the interface region), and wherein the 1, 2, 3, 4, or 5 N-terminal and/or C-terminal amino acid residues, as well as any N-terminal methionine residue, may be present or absent when considering the percent identity.

The disclosure also provides methods for making the heterodimers of the disclosure, and for designing the heterodimers and heterodimer-forming polypeptides.

### DESCRIPTION OF THE FIGURES

FIG. 1A-E. Strategies for the design of asymmetric hetero-oligomeric complexes. (A) Many design efforts have focused on cooperatively assembling symmetric complexes (left) with little subunit exchange. Here instead we sought to create asymmetric hetero-oligomers from stable heterodimeric building blocks, which can modularly exchange subunits (right). (B,C,D) Schematic illustration of properties that can contribute to prevent self-association. (B) Protomers that have a substantial hydrophobic core (right rectangles) are less likely to form stable homo-oligomers than protomers of previously designed heterodimers lacking hydrophobic monomer cores. (C) In beta-sheet extended interfaces, most homodimer states that bury non h-bonding polar edge strand atoms are energetically inaccessible. Potential homodimers are more likely to form via beta sheet extension. These are restricted to only 2 orientations (parallel and antiparallel) and a limited number of offset registers. Arrows and ribbons represent strands and helices, respectively; thin lines indicate hydrogen bonds, stars indicate unsatisfied polar groups. (D) “Cross sectional” schematic view (helices as circles, beta strands as rectangles, star indicates steric clash) By modeling the limited number of beta sheet homodimers across the beta edge strand, structural elements may be designed that specifically block homodimer formation but still allow heterodimer formation. (E) Design workflow: Beta sheet motifs are docked to the edge strands of a library of hydrophobic core containing fold-it scaffolds. Minimized docked strands are incorporated into scaffolds by matching the strands to the scaffold library, yielding docked protein-protein complexes, followed by interface sequence design. Resulting docks are fused rigidly on their terminal helices to a library of DHRs.

FIG. 2A-B. Experimental characterization. (A) Top row, design models of six different heterodimers. Middle row, normalized SEC traces of individual protomers (A, B) and complexes (AB). Bottom row, kinetic binding traces with global kinetic fits of in vitro biolayer interferometry binding assays. (B): Crystal structures (in colors) of the designs LHD29, LHD29A53/B53 and LHD101A53/B4 overlayed on design models. Rectangles in the full models (top row) match the corresponding detailed views (bottom row).

FIG. 3A-F. Design of higher order hetero-oligomers. (A) Schematic overview of experimentally validated rigid fusion proteins comprising a designed helical repeat protein and a protomer for a heterodimer. (B) Schematic representation of the design-free alignment method used to generate bivalent connectors from two of the rigid fusions shown in A. (C) Top: Design model and schematic representation of a heterotrimer comprising the bivalent connector shown in B (“B”), and two of the rigid fusions shown in A (“A” and “C”). Bottom: SEC traces for all possible combinations of the trimer components. (D) Schematic representations of nine different bivalent connectors that were generated as shown in B and experimentally validated as shown in C (see FIG. 15). (E) Schematic representation of experimentally validated higher order assemblies (see FIG. 15-16). (F) Left: overlay of heterohexamer design model and nsEM density. Right: SEC traces of partial and full mixtures of the hexamer components. Absorbance was monitored at 473 nm to follow the GFP-tagged component C.

FIG. 4A-D. Design of branched and closed hetero-oligomeric assemblies. (A) Left: Schematic representation of a trivalent connector (“A”) that can bind three different binding partners (“B”, “C”, “D”). Center: SEC analysis of the trivalent connector, the binding partners, and the full assembly mixture. Right: Overlay of design model and nsEM density of the complex formed by the trivalent connector and all three binding partners. (B) From left to right: Schematic representation of a C3-symmetric “hub” that can bind three copies of one binding partner; SEC analysis of the C3-symmetric “hub” without (“A-”) and with (“AB”) binding partner; overlay of design model) and nsEM density of the C3-symmetric “hub”; overlay of design model and nsEM density of the C3-symmetric “hub” bound to three copies of its binding partner. (C): From left to right: Schematic representation of a C4-symmetric “hub” that can bind four copies of one binding partner; SEC analysis of the C4-symmetric “hub” without (“A-”) and with (“AB”) binding partner; design model (top) and representative nsEM class average (bottom) of the C4-symmetric “hub”; design model (top) and representative nsEM class average (bottom) of the C4-symmetric “hub” bound to 4 copies of the binding partner. (D) From left to right: Schematic representation of a C4-symmetric closed ring comprising two components (“A” and “B”); SEC analysis of the individual ring components (“A-” and “-B”) and the stoichiometric mixture (“AB”); design model of the C4-symmetric ring; representative nsEM class average.

FIG. 5A-B. Dynamically reconfigurable protein assemblies. (A) Exchange experiment in which a pre-assembled trimer (“ABC”) is incubated with a variant of one of the components (“C”). Top: Schematic representation, bottom: SEC traces of trimer mixture before and after addition of component C′. (B) Top: schematic representation of a split luciferase experiment in which two protomers (“A” and “C”) are fused to split luciferase parts. Bottom: Real-time luminescence measurement of two samples containing the mixture “ABC” shown on the left. Bar indicates addition of either buffer or component B′.

FIG. 6. SSM LHD101A yield designs with slower off-rates. Fitted biolayer interferometry kinetic traces comparing LHD101A and mutants of LHD101A. The off-rate becomes lower in the mutants indicating slower dissociation of the complex. On-rates hardly change

FIG. 7. Modification Fold-it scaffolds. Fold-it scaffold 2003333_0006 (left) was expanded with 2 additional helices (middle) on its C-terminus via blueprint-based backbone generation. After backbone generation, the scaffold sequence was designed and the best scaffolds were selected (right) based on per residue Rosetta™ energy and core packing.

FIG. 8A-E. Characterization LHD binding in vitro. A: Designed models heterodimers (top row). Middle row, SEC binding experiments performed on a Superdex™ 75 column. Bottom row, biolayer interferometry kinetic binding traces. B: Convoluted and deconvoluted native mass spectrums of the LHD29 heterodimer. C: Kinetic binding traces from BLI. Equilibrium responses were used to fit equilibrium binding curves D: Equilibrium binding curves of LHDs from biolayer interferometry binding assays with data from C. E: Equilibrium binding curves of unfused LHD101 protomers binding to rigid DHR fusions of LHD101B (DHR4 and 62) and LHD101A (DHR21). Biotinylated unfused protomers were immobilized on streptavidin coated biosensors.

FIG. 9. Oligomeric state of LHD protomers. SEC chromatograms of various LHD protomers titrated at indicated injection concentrations. All experiments were performed on a Superdex™ 200 column except for LHDs 275A, 278A, 284A, 289A, 298A and 317A. These were run on a Superdex™ 75 column.

FIG. 10A-F. Redesign of LHD29. A: Superposition of a redesigned version of LHD29 designated LHD274 and LHD29. Top, atomic view of interface 1 (B) region of LHD29 and interface 2 region (C). Bottom panels, Overlay view of LHD29 and LHD274 at the corresponding region. Thick sticks indicate hydrophobic to polar substitutions. D: SEC Superdex™ 200 titration of LHD29A and LHD274A fused to DHR53 at indicated concentrations. Fusion proteins were chosen for this assay for their enhanced absorbance at 230 nm compared to the much smaller unfused versions. E: SEC Superdex™ 200 titration of LHD29B and LHD274B fused to DHR53 at indicated concentrations. F: Titration of the 29 and 274 complexes.

FIG. 11A-G. Characterization of binding interactions with a split luciferase reporter assay. Protein interactions were characterized by monitoring the reconstitution of split luciferase activity (smBiT:lgBiT) upon binding in buffer (from purified components; A, G-H) or lysate (B-F). A Comparison between the observed association kinetics of LHDs and designed helical hairpins (DHD37, previous work) under pseudo first-order conditions (1 nM vs. 10 nM). Reactions were monitored by taking manual time-points over the course of a week. The data was fitted to a single exponential decay function (solid line; rates are reported in the figure legend). B Example kinetic traces for the association of LHD29 (left) and LHD101 (right) in lysate. Residuals to the fits are shown under each plot, and the rates are reported on top of each plot. C Summary statistics for association reactions performed under pseudo first-order conditions (1 nM vs. 10 nM) in lysate. Values are reported in Table 8. The shaded area indicates the limit of detection of the assay. D Example of equilibrium binding data collected in lysate (shown here for LHD101). Dashed lines are fits to the data, which includes a correction term to account for the intrinsic affinity of the split luciferase components (approximated by the shaded area). The binding curves (excluding the correction) are shown as solid black lines. The fitted K_{d} values are indicated in the figure legend. E Summary statistics for the equilibrium binding experiments performed in lysate. Values are reported in Table 9. F, G Equilibrium binding data (F) and simulation (G) for the ternary complex ABC. The data closely matches the prediction obtained from simulating the system with the affinities of each interface as measured in isolation (K_{d}(LHD101)=5 nM, K_{d}(LHD29)=50 nM), highlighting the modularity and transferability of LHD heterodimers.

FIG. 12A-B. Homodimer docking. A: Example of homodimer docking. Homodimeric interaction most likely will occur on the edgestrand that forms the heterodimer. Strands are docked to the interface edgestrand of a protomer of a given heterodimer. Another copy of the same protomer is then aligned along the docked edgestrand to create a homodimeric docked complex. Most complexes clash indicating homodimerization is unfavorable (top row). Some docks do not clash (bottom row) but have limited interaction surface area making homodimerization unlikely. In some cases homodimer docks i.e. LHD29 have similar interactions energies as the heterodimer (bottom right). These docks are likely to form homodimers. B: Homodimer docking of LHD317 protomers shows that secondary structure elements prevent LHD317A homodimerization via steric occlusion whereas 317B homodimers are more favorable. C: Designed secondary structure elements in both protomors of LHD321 prevent homodimerization

FIG. 13. LHD fusion binding assays. Superdex™ 200 binding assays of LHD fusion proteins.

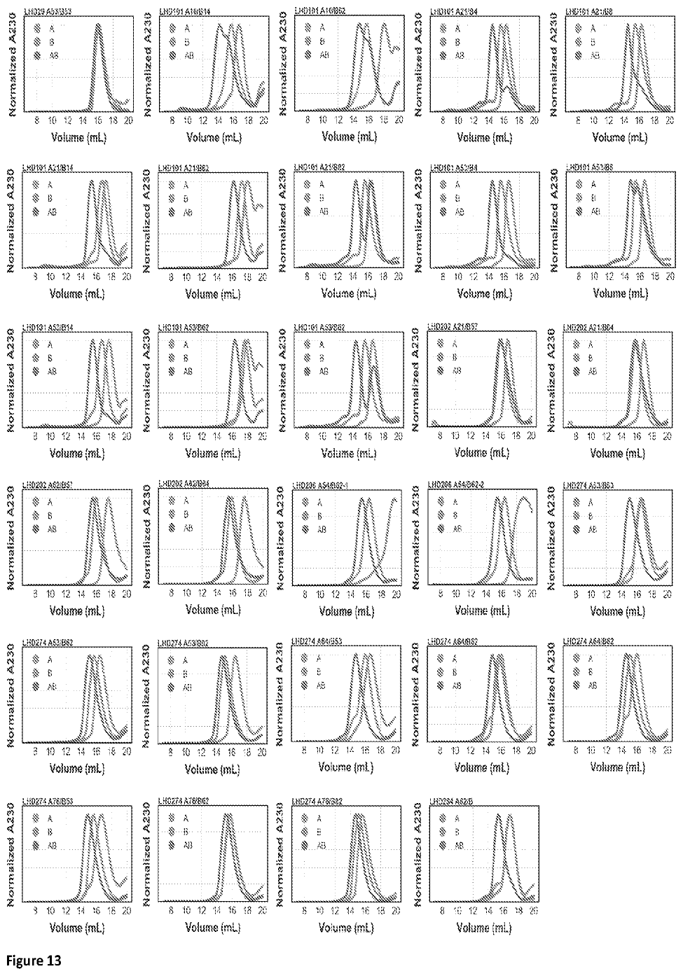

FIG. 14. Models LHD101 fusion complexes. Designed models of all possible 20 complexes involving LHD101 fusions. Combinations with unfused protomers (10 complexes) are not shown.

FIG. 15. SEC binding assays linear hetero-oligomers. Superdex™ 200 chromatograms of various linear assemblies and their control sub-assemblies. Designed models of the target assembly (black chromatogram) are shown right of the graphs

FIG. 16A-D. Negative stain EM class averages and 3D reconstructions hetero-oligomers. A: Heterotrimer (ABC) consisting of LHD274A53 (A), linear connector DFx (B) and LHD317B (C). B: Heteropentamer (ABCDE) consisting of 101B4 (A), DFA0 (B), DF206 (C), DF275A-1 (D) and 275B (E). C: Heterohexamer consisting of 284A82 (A), DF284B (B), DFA0 (C), DF206 (D), DF275A-1 (E) and 275B (F). D: Comparison between designed heteropentamer (left) and the Cull-Rbx1-Skp1-F box^{Skp2} SCF ubiquitin ligase complex (right).

FIG. 17A-D. Non-linearly arranged assemblies. A: Class averages and 3D reconstruction of a branched tetramer (ABCD) consisting of trivalent connector TF10 (A), LHD274A53 (B), LHD317B (C) and LHD101B62 (D). B: SEC and corresponding SDS-PAGE analysis of a branched tetramer consisting of trivalent connector TF3 (A), LHD274A53 (B), LHD275B (C) and LHD101B62. C and D: Class averages and 3D reconstruction of the C3-Hub bound to LHD101A53 and by itself.

FIG. 18A-B. Characterization of C4 hetero-oligomers. A: SEC traces of the C4-symmetric hub at different concentrations without binding partner (left) and with a constant concentration of binding partner (right). Concentrations are given per monomer (5 μM corresponds to 1.25 μM tetramer). B: Schematic representations (left; (C4 hub, binding partner) and negative stain EM class averages (right) of the C4-symmetric hub without (top, center) and with (bottom) binding partner. In absence of the binding partner, the C4 hub exists in equilibrium between a higher order complex (top) and the designed C4 complex (center).

FIG. 19A-B. Characterization of the closed C4-symmetric ring. A: Convoluted and deconvoluted native mass spectrums of the two component C4-symmetrical ring and constituent components. B: Negative stain EM class averages of the closed C4-symmetric ring shown in FIG. 4D

FIG. 20. Biolayer interferometry subunit exchange. Biotinylated LHD101 that is immobilized to streptavidin biosensors binds rigid fusion variant LHD101B62. Biosensors were next dipped into a solution containing equimolar amounts of LHD101B62 and unfused 101B at saturating concentrations. The binding response of this reaction is in between controls indicating subunit exchange takes place.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NO:1-28, or SEQ ID NOS: 1 and 6-28, not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity.

TABLE 1
Sequences of polypeptide-forming heterodimers, shown together with
their heterodimers pair. Interface residues are lower case,
non-interface residues areupper case

| SEQ ID | |
|---|---|
| NO: | Sequence |
| | LHD101.pdb |
| 1 | chainA |
| | GRQEKVLKSIEETVRKMGVTMETHRSGNEVKVVIKGLHikQqrqLyrDVrETSkKQG |
| | VeTeievegdTVTIVVRE |
| 2 | chainA >LHD101A_Q42M |
| | GRQEKVLKSIEETVRKMGVTMETHRSGNEVKVVIKGLHIKQMRQLYRDVRETSKKQG |
| | VETEIEVEGDTVTIVVRE |
| 3 | chainA >LHD101A_R43V |
| | GRQEKVLKSIEETVRKMGVTMETHRSGNEVKVVIKGLHIKQQVQLYRDVRETSKKOG |
| | VETEIEVEGDTVTIVVRE |
| | chainA >LHD101A_V69A |
| | GRQEKVLKSIEETVRKMGVTMETHRSGNEVKVVIKGLHIKQQRQLYRDVRETSKKQG |
| | VETEIEVEGDTQTIVVRE |
| 5 | chainA >LHD101A_T70W |
| | GRQEKVLKSIEETVRKMGVTMETHRSGNEVKVVIKGLHIKQQRQLYRDVRETSKKOG |
| | VETEIEVEGDTVWIVVRE |
| 6 | chainB: |
| | GRQEKVLKSIEETVRKMGVTMETHRSGNEVKVVIKGLHeSQqeqLleDvlrTaeKOG |
| | VrvrirfkgDTVTIvVRE |
| | LHD202.pdb |
| 7 | chainA: |
| | GRQEKVLKSIEETVRKMGVTMETHRSGNkVKVVIKGLHESQQEQLrKDvhETlrkqg |
| | vvavtqkhGDTVtiyVte |
| 8 | chainB: |
| | svefhivniSEEQRQRIEEYVRRISKKEGTEVRFEKRDGeLtIEVKNlHeKRlqEil |
| | eYieRVnk |
| | LHD206.pdb |
| 9 | chainA: |
| | TDELLERLRQLFEELHERGTEIVVEvHiNGrkteievqgidKrlLkiiLeviReeIE |
| | REGSSEVEVNVHSGGQTWTFNEK |
| 10 | chainB: |
| | GRQEKVLKSIEETVRKMGVTMETHRSGNEVKVVIKGLHkSQqeQLlkDVlkTanKQg |
| | vnvhisfrgDTVTIrVrE |
| | LHD274.pdb |
| 11 | chainA: |
| | ttnfhlingsEEaRQRIEEYVRRISKKEGTEVHFEKsdgtLeirVKNLHEKReREik |
| | EYieRVll |
| 12 | chainB: |
| | nthfivvhgSEEaRQRaEEYVRRISKKEGTEVRFEKkdgllsievKNISeERqrEiq |
| | eYlqRvqk |
| | LHD275.pdb |
| 13 | chainA: |
| | GRQEKVLKSIEETVRKMGVEMLTFRAGNAVIVVIRGLHpeQakqLlrDvsqtahkQg |
| | vtvtltfhgDVVfILVLVGASEEEqKHMqERiqELaRIIHEAKRRGVSEEQLREIAE |
| | KMAKEIQEWG |
| 14 | chainB: |
| | DVEWRYTNISeETqqkSaeFvleIalrAgtgvtfttrqgElqIqVhNLDELLAIAML |
| | CYTLGLLLGDHRVQELAKRAVEAWERGDEERVKKLLIEALKRLVETAEEVVRERPGS |
| | NLAKLALEIILRAAEALARAEDPESLKEAVKAAEKVVREQPGSNLAKKALEIILRAA |
| | EELAKLPDPEALKEAVKAAEKVVREQPGSELAKKALEIIERAAEELKKSPDPEAQKE |
| | AKKAEQKVREERPGS |
| | LHD278.pdb |
| 15 | chainA: |
| | GRQEKVLKSIEETVRKMGVRMLTHRGGNAVIVVIEGLHpSQikQLmqDVikTakKQg |
| | vtvtitvsgDIVVIMVVVGASdEEqeEarRLvqEIaRALqEAKRKGANEEQLEQLLR |
| | ELLERAEREG |
| 16 | chainB: |
| | TVTFDITNIDwkSaeLImlAVydIaqQEgTdvtfsfkeGeLqItVkNLHEKWKRLIE |
| | MLIEACRRAQDPDPESLKEAVRIAEELVRLHPGNPLARAALKVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |
| | LHD284.pdb |
| 17 | chainA: |
| | TDELLERLRQLFEELHERGtiIiVEVHINGErqtkylilapKEeLKKhLERIREKIE |
| | REGSSEVEVkVtSggttWTFNEK |
| 18 | chainB: |
| | phqfyvyqiDEHVAQLIEKFVRDISRREGTEVRFEKRDGqLEIEVKNLHeAQaIAig |
| | IYimILILHQSGTSEDEIAEEIAklIkgfiehLKreGSSYEVICEAVAAAVAAIVKA |
| | LKGCGTSEDEIAEIVARVISEVIRTLKESGSSYEVICECVARIVAEIVQALKESGTS |
| | EDEIAEIVARVISEVIRTLKESGSSYEVIKECVQRIVEEIVEALKRSGTSEDEINEI |
| | VRRVKSEVERTLKESGSS |
| | LHD289.pdb |
| 19 | chainA: |
| | GRQEKVLKSIEETVRKMGVTMLTHRHGNVVFVVILGLHkqQalQLlrDvhrTahKQg |
| | VtlsitfsgDIVVIAVTVGASEEEkKEVrKIvkEIaKQLrHAETEEEAKEIVORVIE |
| | EWQEEG |
| 20 | chainB: |
| | TVTFDITNIShEAieIIlygVlgIaamEgTevtfhserGQLqIeVkNLHEKQKRNIE |
| | KLIEAALRAQSPDPEDLKEAVRIAEELVRAHPGTpLAHAALQVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |
| | LHD29.pdb |
| 21 | chainA: |
| | twqwvliniSEEaRQRIEEYVRRISKKEGTEVHFEKddgvLhIrVKNLHEKRaREIh |
| | EYakRVil |
| 22 | chainB: |
| | ssifllsnvSEEARQRaEEYVRRISKKEGTEVRFEKdDgfltiEvKNISeERlrEia |
| | eYlwRvav |
| | LHD298.pdb |
| 23 | chainA: |
| | GRQEKVLKSIEETVRKMGVTMETHRSGntVKVVIKGLHESQQEQLhKDveETvqkeg |
| | vfvlvshhGDTVtIqVye |
| 24 | chainB: |
| | shsfilgqaSEEARQEIEEVVEAISRKLGTEVRFEKkDgtLhIEVKNIHdEYaqLia |
| | dAilLiiLAQESDDSEAKKVARLALEIVAQLPNTELAHEALKLAEEALKSTDSEALK |
| | VVYLALRIVQQLPDTELAREALELAKEAVKSTDQEALKSVYEALQRVODKPNTEEAR |
| | ESLERAKEDVKSTD |
| 25 | LHD317.pdb |
| | chainA: |
| | GRQEKVLKSIEETVRKMGVRMLTHRGGNAVIVVIEGLHpSQaeQLlrDvhrtakkqg |
| | vtvhlvftgdIVVIMVVVGASEEEqEEMhRLvrEIaeALhEAKRKGANEEQLEQLLR |
| | ELLERAEREG |
| 26 | chainB: |
| | DVEWRFTNVSeEEqeKLarFVlqVaqlAgtqvifttrpgElrIRVHNLDELLALAIE |
| | LYAQGLRLGDKHVQHLAKKAIEAILRGDRKLARFLLEAARAMSRATERPGSNLAKKA |
| | LEEILRLAEELAKDPDPESLKAAVHCAEFVVRYQPGSNLAKKALEIILRAAEELAKL |
| | PDPEALKEAVKAAEKVVREQPGSELAKKALEIIERAAEELKKSPDPEAQKEAKKAEQ |
| | KVREERPGS |
| | LHD321.pdb |
| 27 | chainA: |
| | TKEELKRAIEEAHRKGDKEKLKEVIKRAQEEGDEEVYREAIQALAKLIAEEAGVDDV |
| | RVEVHNGrVRLEIRgqSqAvvrVatevvtelgklgirvtvqlg |
| 28 | chainB: |
| | TVTFDITNIDdkStkliatavihIagrEgttvhfqghdGQlEIEVKNLHEKWKRLIE |
| | MLIEACRRAQDPDPESLKEAVRIAEELVRLHPGNmLAeAALKVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |

As described in the examples that follow, the inventors employed a set of implicit negative design principles to generate beta sheet mediated heterodimers, which enable the generation of a wide variety of structurally well-defined asymmetric assemblies. Crystal structures of the heterodimers are very close to the design models, and unlike previously designed orthogonal heterodimer sets, the subunits are stable, folded and monomeric in isolation and rapidly assemble upon mixing. Rigid fusion of individual heterodimer halves to repeat proteins yields central assembly hubs that can bind two or three different proteins across different interfaces. We use these connectors to assemble linearly arranged hetero-oligomers with up to 6 unique components, branched hetero-oligomers, closed C4-symmetric two-component rings, and hetero-oligomers assembled on a cyclic homo-oligomeric central hub, and demonstrate such complexes can readily reconfigure through subunit exchange. Thus, the polypeptides can be used, for example, to generate asymmetric reconfigurable protein systems. Such systems may, for example, include fusion to target proteins of interest to co-localize and position multiple copies of the same target fusion for any suitable purpose such as to target multiple copies of therapeutic proteins of interest for therapeutic treatment.

In one embodiment, 1, 2, 3, 4, 5, 6, 7, 8, 9, 10, 11, 12, 13, 14, 15, 16, 17, 18, 19, 20, 21, or all of identified interface amino acid residues in Table 1 are identical at that residue position to the reference polypeptide. Interface residues are shown in lower case in Table 1 for SEQ ID NOS:1 and 6-28, while the interface residues in SEQ ID NOS:2-5 are at the same positions as the interface residues in SEQ ID NO:1, as SEQ ID NOS: 2-5 are point mutations relative to SEQ ID NO:1 (specific point mutation identified in the name of the sequences).

In another embodiment, 1, 2, 3, 4, or 5 N-terminal and/or C-terminal amino acid residues are not included when determining the percent identity relative to the reference polypeptide. In another embodiment, all residues are included when determining the percent identity relative to the reference polypeptide.

In one embodiment, the polypeptides may comprise an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1, including 1, 2, 3, or all 4 of the following mutations relative to SEQ ID NO:1: Q42M, R43V, V69Q, and T70W.

In a further embodiment, amino acid substitutions relative to the reference polypeptide are conservative substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the disclosure provides heterodimer-forming polypeptides, comprising the amino acid sequence of any one of SEQ ID NOS:29-33, 35-55, and 190-191 or comprising the amino acid sequence of any one of SEQ ID NOS:29, 35-55, and 190-191, in which:
(a) all interface residues identified for a single heterodimer-forming polypeptide disclosed in Table 1, and
(b) any amino acid at each position of the of the same heterodimer-forming polypeptide that is identified as not being an interface residue;
wherein any functional domains added fused to the polypeptides (whether N-terminal, C-terminal, or internal other than within the interface region), and wherein the 1, 2, 3, 4, or 5 N-terminal and/or C-terminal amino acid residues may be present or absent.

As demonstrated by fusion of heterodimer-forming domains to designed helical repeat proteins (see examples), such fusion proteins retain the binding properties of the original heterodimer-forming components as long as the interface residues remain unchanged.

Moreover, there are many changes to the sequence in the core of the heterodimer-forming domains or in the non-interface surface regions that can be expected to have no effect on the heterodimerization properties. It can thus be concluded that the heterodimerization properties are directly linked to the residue identities at the interface.

In this embodiment, the interface residues of the heterodimer-forming polypeptides are held constant, while all other residues in the polypeptide are variable. By way of example, LHD101.pdb chain A (SEQ ID NO:1) is disclosed herein as one member of a heterodimer forming polypeptide pair. The LHD101.pdb chain A sequence is shown below

LHD101.pdb
chainA:
(SEQ ID NO: 1)
GRQEKVLKSIEETVRKMGVTMETHRSGNEVKVVIKGLHi
kQqrqLyrDVrETSkKQGVeTeievegdTVTIVVRE

In this embodiment, the corresponding sequence would be as follows, wherein X is any amino acid residue

chainA:
(SEQ ID NO: 29)
XXXXXXXXXXXXXXXXXXXXXXXXXXXXXXXXXXXXXX
ikXqrqXyrXXXXXXXXXXXeXeievegdXXXXXXXX

All sequences according to this embodiment are shown in Table 2.

TABLE 2

| SEQ ID | |
|---|---|
| NO | Sequence |
| | LHD101.pdb |
| 29 | chainA: |
| | XXXXXXXXXXXXXXXXXXXXXXXXXXXXXXXXXXXXXXikXqrqXyrXXrX |
| | XXkXXXXeXeievegdXXXXXXXX |
| 30 | chainA Q42M: |
| | XXXXXXXXXXXXXXXXXXXXXXXXXXXXXXXXXXXXXXikXmrqXyrXXrX |
| | XXkXXXXeXeievegdXXXXXXXX |
| 31 | chainA R43V: |
| | XXXXXXXXXXXXXXXXXXXXXXXXXXXXXXXXXXXXXXikXqvqXyrXXrX |
| | XXkXXXXeXeievegdXXXXXXXX |
| 32 | chainA Q42M and R43V: |
| | XXXXXXXXXXXXXXXXXXXXXXXXXXXXXXXXXXXXXXikXmvqXyrXXrX |
| | XXkXXXXeXeievegdXXXXXXXX |
| 33 | chainB |
| | XXXXXXXXXXXXXXXXXXXXXXXXXXXXXXXXXXXXXXeXXqeqXleXvlr |
| | XaeXXXXrvrirfkgXXXXXvXXX |
| | LHD202.pdb |
| 190 | chainA: |
| | XXXXXXXXXXXXXXXXXXXXXXXXXXXXkXXXXXXXXXXXXXXXXrXXvhX |
| | XlrkqgvvavtqkhXXXXtiyXte |
| 191 | chainB |
| | svefhivniXXXXXXXXXXXXXXXXXXXXXXXXXXXXXeXtXXXXXlXeXX |
| | lqXileXieXXnk |
| | LHD206.pdb |
| 35 | chainA: |
| | XXXXXXXXXXXXXXXXXXXXXXXXXvXiXXrkteievqgidXr1XkiiXev |
| | iXeeXXXXXXXXXXXXXXXXXXXXXXXXX |
| 36 | chainB: |
| | XXXXXXXXXXXXXXXXXXXXXXXXXXXXXXXXXXXXXXkXXqeXXlkXXlk |
| | XanXXgvnvhisfrgXXXXXrXrX |
| | LHD274.pdb |
| 37 | chainA: |
| | ttnfhlingsXXaXXXXXXXXXXXXXXXXXXXXXXXsdgtXeirXXXXXXX |
| | XeXXikEXieXXll |
| 38 | chainB: |
| | nthfivvhqXXXaXXXaXXXXXXXXXXXXXXXXXXXkdgllsievXXlXeX |
| | XqrXiqeXlqXvqk |
| | LHD275.pdb |
| 39 | chainA: |
| | XXXXXXXXXXXXXXXXXXXXXXXXXXXXXXXXXXXXXXpeXakqXlrXvsq |
| | tahkXgvtvtltfhgXXXfIXXXXXXXXXXqXXXqXXiqXXaXXXXXXXXXXXXXXX |
| | XXXXXXXXXXXXXXXX |
| 40 | chainB: |
| | XXXXXXXXXXeXXqqkXaeXvleXalrXgtgvtfttrqgXlqXqXhXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXX |
| | LHD278.pdb |
| 41 | chainA: |
| | XXXXXXXXXXXXXXXXXXXXXXXXXXXXXXXXXXXXXXpXXikXXmqXXik |
| | XakXXgvtvtitvsgXXXXXXXXXXXXdXXqeXarXXvqXIaXXXqXXXXXXXXXXX |
| | XXXXXXXXXXXXXXXX |
| 42 | chainB: |
| | XXXXXXXXXXwkXaeLImlXXydIaqQEgXdvtfsfkeXeXqXtXkXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXX |
| | LHD284.pdb |
| 43 | chainA: |
| | XXXXXXXXXXXXXXXXXXXtiIiXXXXXXXXrqtkylilapXXeXXXhXXX |
| | XXXXXXXXXXXXkXtSggttXXXXXX |
| 44 | chainB: |
| | phqfyvyqiXXXXXXXXXXXXXXXXXXXXXXXXXXXXXXqXXXXXXXXXeX |
| | XaXXigIYimXXlXXXXXXXXXXXXXXXXklIkqfiehXXreXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXX |
| | LHD289.pdb |
| 46 | chainA: |
| | XXXXXXXXXXXXXXXXXXXXXXXXXXXXXXXXXXXXXXkqXa1XXlrXvhr |
| | XahXXgXtlsitfsgXXXvXXXXXXXXXXXkXXXrXIvkXXaXXXrXXXXXXXXXXX |
| | XXXXXXXXXXXX |
| 47 | chainB: |
| | XXXXXXXXXXhXXieXXlygXlgXaamXgXevtfhserXXXqXeXkXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXX |
| | LHD29.pdb |
| 48 | chainA: |
| | twqwvliniXXXaXXXXXXXXXXXXXXXXXXXXXXXddgvXhIrXXXXXXX |
| | XaXXXhXXakXXil |
| 49 | chainB: |
| | ssifllsnvXXXXXXXaXXXXXXXXXXXXXXXXXXXdXgfltiXvXXlXeX |
| | XlrXiaeXlwXvav |
| | LHD298.pdb |
| 50 | chainA: |
| | XXXXXXXXXXXXXXXXXXXXXXXXXXXntXXXXXXXXXXXXXXXXhXXveX |
| | XvqkegvfvlvshhXXXXtXqXye |
| 51 | chainB: |
| | shsfilgqaXxxxXXXXXXXXXXXXXXXXXXXXXXXXXgtXhXXXXXlXfX |
| | XaqXiadXilXiiXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXX |
| | LHD317.pdb |
| 52 | chainA: |
| | XXXXXXXXXXXXXXXXXXXXXXXXXXXXXXXXXXXXXXXXaeXXlrXvhr |
| | takkqgvtvhlvftgdXXXXXXXXXXXXXXqXXXhXXvrXIaeXLhXXXXXXXXXXX |
| | XXXXXXXXXXXXXXXX |
| 53 | chainB: |
| | XXXXXXXXXXeXXqeXXarXXlqXaqlXgtqvifttrpgXlrXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXX |
| | LHD321.pdb |
| 54 | chainA: |
| | XXXXXXXXXXXXXXXXXXXXXXXXXXXXXXXXXXXXXXXXXXXXXXXXXXX |
| | XXXXXXXXXXXXXXXXXXgqXqXvvrXatevvtelgklgirvtvqlg |
| 55 | chainB: |
| | XXXXXXXXXXdkXtkliatavihXagrXgttvhfqghdXXlXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXX |

In another embodiment, the disclosure provides heterodimer-forming polypeptides, comprising the amino acid sequence of any one of SEQ ID NOS:56-77 and 191, or comprising the amino acid sequence of any one of SEQ ID NOS: 56, 60-77, and 191 in which the protein domain that includes all of the identified interface residues for a single heterodimer-forming polypeptide disclosed herein, and wherein X is any amino acid residue.

In this embodiment, the corresponding sequence for LHD101.pdb chain A (SEQ ID NO: 1) would be as follows, where X is any amino acid residue.

(SEQ ID NO: 56)
ikXqrqXyrXXXXXXXXXXXeXeievegd

All sequences according to this embodiment are shown in Table 3.

TABLE 3

| SEQ ID | |
|---|---|
| NO | Sequence |
| | LHD101.pdb |
| 56 | chainA: ikXqrqXyrXXrXXXXXXXXeXeievegd |
| 57 | chainA Q42M: ikXmrqXyrXXrXXXXXXXXeXeievegd |
| 58 | chainA R43V: ikXqvqXyrXXXXXXXXXXXeXeievegd |
| 59 | chainA Q42M and R43V: ikXmvqXyrXXXXXXXXXXXeXeievegd |
| 60 | chainB eXXqeqXleXvlrXaeXXXXrvrirfkgXXXXXv |
| | LHD202.pdb |
| 61 | chainA: |
| | kXXXXXXXXXXXXXXXXrXXvhXXIrkqgvvavtqkhXXXXtiyXte |
| 191 | chainB |
| | svefhivniXXXXXXXXXXXXXXXXXXXXXXXXXXXXXXeXtXXXXX |
| | IXeXXlqXileXieXXnk |
| | LHD206.pdb |
| 62 | chainA: VXXvXiXXrkteievqgidXr1XkiiXeviXee |
| 63 | chainB: kXXqeXXlkXXlkXanXXgvnvhisfrgXXXXXrXr |
| | LHD274.pdb |
| 37 | chainA: |
| | ttnfhlingsXXaXXXXXXXXXXXXXXXXXXXXXXXsdgtXeirXXXXXXX |
| | XeXXikEXieXXll |
| 38 | chainB: |
| | nthfivvhqXXXaXXXaXXXXXXXXXXXXXXXXXXXkdgllsievXXlXeX |
| | XqrXiqeXlqXvqk |
| | LHD275.pdb |
| 64 | chainA: |
| | peXakqXlrXvsqtahkXgvtvtltfhgXXXfIXXXXXXXXXXqXXXqXXi |
| | qXXa |
| 65 | chainB: eXXqqkXaeXvleXalrXgtgvtfttrqgXlqXqXh |
| | LHD278.pdb |
| 66 | chainA: |
| | pXXikXXmqXXikXakXXgvtvtitvsgXXXXXXXXXXXXdXXqeXarXXv |
| | qXIaXXXq |
| 67 | chainB: wkXaeLImlXXydIaqQEgXdvtfsfkeXeXqXtXk |
| | LHD284.pdb |
| 68 | chainA: |
| | tiIiXXXXXXXXrqtkylilapXXeXXXhXXXXXXXXXXXXXXXXXXkXtS |
| | ggtt |
| 69 | chainB: |
| | phqfyvyqiXXXXXXXXXXXXXXXXXXXXXXXXXXXXXXqXXXXXXXXXeX |
| | XaXXigIYimXXlXXXXXXXXXXXXXXXXklIkgfiehXXre |
| | LHD289.pdb |
| 70 | chainA: |
| | kqXa1XXlrXvhrXahXXgXtlsitfsgXXXvXXXXXXXXXXXXXXXrXIv |
| | kXXaXXXr |
| 71 | chainB: hXXieXXlygXlgXaamXgXevtfhserXXXqXeXk |
| | LHD29.pdb |
| 48 | chainA: |
| | twqwvliniXXXaXXXXXXXXXXXXXXXXXXXXXXXddgvXhIrXXXXXXX |
| | XaXXXhXXakXXil |
| 49 | chainB: |
| | ssifllsnvXXXXXXXaXXXXXXXXXXXXXXXXXXXdXgfltiXvXXlXeX |
| | XlrXiaeXlwXvav |
| | LHD298.pdb |
| 72 | chainA: |
| | ntXXXXXXXXXXXXXXXXhXXveXXvqkegvfvlvshhXXXXtXqXye |
| 73 | chainB: |
| | shsfilgqaXXXXXXXXXXXXXXXXXXXXXXXXXXXXXgtXhXXXXXlXfX |
| | XaqXiadXilXii |
| | LHD317.pdb |
| 74 | chainA: |
| | pXXaeXXlrXvhrtakkqgvtvhlvftgdXXXXXXXXXXXXXXqXXXhXXv |
| | rXIaeXLh |
| 75 | chainB: eXXqeXXarXXlqXaqlXgtqvifttrpgXlr |
| | LHD321.pdb |
| 76 | chainA: rXXXXXXgqXqXvvrXatevvtelgklgirvtvqlg |
| 77 | chainB: |
| | dkXtkliatavihXagrXgttvhfqghdXXl |

In another embodiment, the disclosure comprises fusion proteins, comprising the polypeptide of any embodiment or combination of embodiments disclosed herein (the “first” polypeptide), and a second polypeptide, optionally including an amino acid linker between the first polypeptide and the second polypeptide. As described herein, since the unfused heterodimer-forming monomers are small (between 7 and 15 kDa without DHR or tags), they can be readily fused to target proteins of interest.

In this embodiment, the first polypeptide may be N-terminal to the second polypeptide, or may be C-terminal to the second polypeptide. The second polypeptide may be any polypeptide of interest, including but not limited to a connector polypeptide (i.e.: a linker or more specific polypeptide to join the monomer to other polypeptides of interest) or a functional polypeptide of interest (including but not limited to therapeutic polypeptides, diagnostic polypeptides, repeat polypeptides, structural polypeptides, detectable polypeptides, receptor-ligand systems etc.) An amino acid linker may be present between the first polypeptide and the second polypeptide; when present, the linker may be any length and amino acid composition as appropriate for an intended use.

In one embodiment, the second polypeptide comprises a repeat polypeptide. Any suitable repeat polypeptide may be used that consists of repeating subunits of two or three helices connected by structured loops. Rigid fusion of individual heterodimer halves to repeat proteins yields central assembly hubs that can bind two or three different proteins across different interfaces. In exemplary embodiments, the second polypeptide repeat protein may comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:78-89, the sequences of which are provided in Table 4.

TABLE 4

| SEQ | | |
|---|---|---|
| ID NO | name | sequence |
| 78 | DHR4 | YEDECEEKARRVAEKVERLKRSGTSEDEIAEEVAREISEVIRTLKESGSSYEVICEC |
| | | VARIVAEIVEALKRSGTSEDEIAEIVARVISEVIRTLKESGSSYEVICECVARIVAE |
| | | IVEALKRSGTSEDEIAEIVARVISEVIRTLKESGSSYEVIKECVQRIVEEIVEALKR |
| | | SGTSEDEINEIVRRVKSEVERTLKESGSS |
| 79 | DHR8 | DEMKKVMEALKKAVELAKKNNDDEVAREIERAAKEIVEALRENNSDEMAKV |
| | | MLALAKAVLLAAKNNDDEVAREIARAAAEIVEALRENNSDEMAKVMLALAK |
| | | AVLLAAKNNDDEVAREIARAAAEIVEALRENNSDEMAKKMLELAKRVLDAA |
| | | KNNDDETAREIARQAAEEVEADRENNS |
| 80 | DHR9 | YEDEAEEKARRVAEKVERLKRSGTSEDEIAEEVAREISEVIRTLKESGSSYEVIAEI |
| | | VARIVAEIVEALKRSGTSEDEIAEIVARVISEVIRTLKESGSSYEVIAEIVARIVAE |
| | | IVEALKRSGTSEDEIAEIVARVISEVIRTLKESGSSYEVIKEIVQRIVEEIVEALKR |
| | | SGTSEDEINEIVRRVKSEVERTLKESGSS |
| 81 | DHR10 | SSEKEELRERLVKIVVENAKRKGDDTEEAREAAREAFELVREAAERAGIDSSEVLEL |
| | | AIRLIKEVVENAQREGYDISEAARAAAEAFKRVAEAAKRAGITSSEVLELAIRLIKE |
| | | VVENAQREGYDISEAARAAAEAFKRVAEAAKRAGITSSETLKRAIEEIRKRVEEAQR |
| | | EGNDISEAARQAAEEFRKKAEELKRRGDV |
| 82 | DHR14 | SEEVNERVKQLAEKAKEATDKEEVIEIVKELAELAKQSTDSELVN |
| | | EIVKQLAEVAKEATDKELVIYIVKILAELAKQSTDSELVNEIVKQ |
| | | LAEVAKEATDKELVIYIVKILAELAKQSTDSELVNEIVKQLEEVA |
| | | KEATDKELVEHIEKILEELKKQSTD |
| 83 | DHR21 | SEKEKVEELAQRIREQLPDTELAREAQELADEARKSDDSEAL |
| | | KVVYLALRIVQQLPDTELAREALELAKEAVKSTDSEALKVVY |
| | | LALRIVQQLPDTELAREALELAKEAVKSTDQEALKSVYEALQ |
| | | RVQDKPNTEEARESLERAKEDVKSTD |
| 84 | DHR52 | CEDRKEKIRELERKARENTGSDEARQAVKEIARIAKEALEEGCCDTAK |
| | | EAIQRLEDLARDYSGSDVASLAVKAIAKIAETALRNGCCDTAKEAIQR |
| | | LEDLARDYSGSDVASLAVKAIAKIAETALRNGCKETAEEAIKRLRELA |
| | | EDYKGSEVAKLAEEAIERIEKVSRERGQ |
| 85 | DHR53 | NDEKEKLKELLKRAEELAKSPDPEDLKEAVRLAEEVVRERPGSNLAKK |
| | | ALEIILRAAEELAKLPDPEALKEAVKAAEKVVREQPGSNLAKKALEII |
| | | LRAAEELAKLPDPEALKEAVKAAEKVVREQPGSELAKKALEIIERAAE |
| | | ELKKSPDPEAQKEAKKAEQKVREERPGS |
| 86 | DHR62 | NDEKRKRAEKALQRAQEAEKKGDVEEAVRAAQEAVRAAKESGDNDVLR |
| | | KVAEQALRIAKEAEKQGNVEVAVKAARVAVEAAKQAGDNDVLRKVAEQ |
| | | ALRIAKEAEKQGNVEVAVKAARVAVEAAKQAGDQDVLRKVSEQAERIS |
| | | KEAKKQGNSEVSEEARKVADEAKKQTGD |
| 87 | DHR64 | PEDELKRVEKLVKEAEELLRQAKEKGSEEDLEKALRTAEEAAREAKKVLEQAEKEGDPEVA |
| | | LRAVELVVRVAELLLRIAKESGSEEALERALRVAEEAARLAKRVLELAEKQGDPEVALRAV |
| | | ELVVRVAELLLRIAKESGSEEALERALRVAEEAARLAKRVLELAEKQGDPEVARRAVELVK |
| | | RVAELLERIARESGSEEAKERAERVREEARELQERVKELREREGD |
| 88 | DHR76 | PELEEWIRRAKEVAKEVEKVAQRAEEEGNPDLRDSAKELRRAVEEAIEEAKKQGNPELVEW |
| | | VARAAKVAAEVIKVAIQAEKEGNRDLFRAALELVRAVIEAIEEAVKQGNPELVEWVARAAK |
| | | VAAEVIKVAIQAEKEGNRDLFRAALELVRAVIEAIEEAVKQGNPELVERVARLAKKAAELI |
| | | KRAIRAEKEGNRDERREALERVREVIERIEELVRQGN |
| 89 | DHR82 | DEEVQEAVERAEELREEAEELIKKARKTGDPELLRKALEALEEAVRAVEEAIKRNPDNDEAV |
| | | ETAVRLARELKKVAEELQERAKKTGDPELLKLALRALEVAVRAVELAIKSNPDNDEAVETAV |
| | | RLARELKKVAEELQERAKKTGDPELLKLALRALEVAVRAVELAIKSNPDNEEAVETAKRLAE |
| | | ELRKVAELLEERAKETGDPELQELAKRAKEVADRARELAKKSNPNN |

In another embodiment, the fusion proteins may comprise a third functional polypeptide C-terminal to the second polypeptide, or N-terminal to the first polypeptide, wherein an amino acid linker is optionally present between the second polypeptide and the third polypeptide, or between the third polypeptide and the first polypeptide.

The third polypeptide may be any polypeptide suitable for an intended purpose. In various embodiments, the third polypeptide may include but is not limited to therapeutic polypeptides, diagnostic polypeptides, detectable polypeptides, receptor-ligand systems, etc.

Exemplary fusion proteins according to these embodiments are listed in Table 5.

Thus, in another embodiment, exemplary fusion proteins comprise an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 90-189 and 196-199, or SEQ ID NOS: 90-189, not including any functional domains added (whether N-terminal, C-terminal, or internal other than within the interface region), and wherein the 1, 2, 3, 4, or 5 N-terminal and/or C-terminal amino acid residues, as well as any N-terminal methionine residue, may be present or absent when considering the percent identity. In Table 5, some sequences are provided twice: once with His tags and other optional residues, and once without optional residues.

TABLE 5

| | |
|---|---|
| SEQ ID | |
| NO | Protein |

name: C4-Hub; alt. name: C4 53; type: Cn

| | |
|---|---|
| 90 | GNTHFIVVHGSEDAAQLAEELVREISKKEGTEVRFEKKDGLLSIEVKNLSEERQREI |
| | QKALQLVQDVANAERVVRERPGSNLAKKALEIILRAAEELAKLDLKASLKAAVRAAE |
| | KVVREQPGSELAKKALEIILRAAEELAKLPDPEALHEAVRAAEHVVRSQPGSEAAKE |
| | ALRIIQEAAELLKESPDPTAIIRAARALLKIARTTGDEEAAKEAIEAAKKAADLARE |
| | RGDDELVCEALALLVAAQVELLKQQGTSAVEIAKIVARVISEVIRTLKEKGSSYEVI |
| | CECVARIVAEIVEALKRSGTSAAIIALIVALVISEVIRTLKESGSSFEVILECVIRI |
| | VLEIIEALKRSGTSEQDVMLIVMAVLLVVLATLQLSGSGSLEHHHHHH |
| 91 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSELAKKALEIILRAAEELAKLPDPEALHEAVRAAEHVVRSQPGSEAAKEA |
| | LRIIQEAAELLKESPDPTAIIRAARALLKIARTTGDEEAAKEAIEAAKKAADLARER |
| | GDDELVCEALALLVAAQVELLKQQGTSAVEIAKIVARVISEVIRTLKEKGSSYEVIC |
| | ECVARIVAEIVEALKRSGTSAAIIALIVALVISEVIRTLKESGSSFEVILECVIRIV |
| | LEIIEALKRSGTSEQDVMLIVMAVLLVVLATLQLSGS |

name: DFA-1; alt. name: 274A_53_−1_101A; type: Connector bivalent

| | |
|---|---|
| 92 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAAALANLPDPESRKEADKAADKVRREQPGSELAVVAAIISAVARMGVKME |
| | LHPSGNEVKVVIKGLHIKQQRQLYRDVREAAKKAGVEVEIEVEGDTVTIVVRGGSHH |
| | WGSGSHHHHHH |
| 93 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAAALANLPDPESRKEADKAADKVRREQPGSELAVVAAIISAVARMGVKME |
| | LHP SGNEVKVVIKGLHIKQQRQLYRDVREAAKKAGVEVEIEVEGDTVTIVVRG |

name: DF0; alt. name: 29A_53_101A; type: Connector bivalent

| | |
|---|---|
| 94 | MTWQWVLINISEEARQLIEKAVRAISKKEGTEVHFEKDDGVLHIRVKNLHEKRAREI |
| | HKVAKLILEVAAAERIVRERPGSNLAKKALEIILRAAEELAKADVDAALEAAVRAAE |
| | KVVREQPGSNLAKKALEIILRAAEELAKLPDPEALKEAVKAAEKVVREQPGSNLAKK |
| | ALEIILRAAAALANLPDPESRKEADKAADKVRREQPGSELAVVAAIISAVARMGVKM |
| | ELHPSGNEVKVVIKGLHIKQQRQLYRDVREAAKKAGVEVEIEVEGDTVTIVVRGSGG |
| | SGSHHWGLEHHHHHH |
| 95 | TWQWVLINISEEARQLIEKAVRAISKKEGTEVHFEKDDGVLHIRVKNLHEKRAREIH |
| | KVAKLILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAAALANLPDPESRKEADKAADKVRREQPGSELAVVAAIISAVARMGVKME |
| | LHPSGNEVKVVIKGLHIKQQRQLYRDVREAAKKAGVEVEIEVEGDTVTIVVRG |

name: DFA0; alt. name: 274A_53_0_101A; type: Connector bivalent

| | |
|---|---|
| 96 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRGGSHHWGSGSHHHHHH |
| 97 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRG |

name: DFB-1; alt. name: 274B_53_−1_101A; type: Connector bivalent

| | |
|---|---|
| 98 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSNLAKKA |
| | LEIILRAAAALANLPDPESRKEADKAADKVRREQPGSELAVVAAIISAVARMGVKME |
| | LHPSGNEVKVVIKGLHIKQQRQLYRDVREAAKKAGVEVEIEVEGDTVTIVVRGGSHH |
| | WGSGSHHHHHH |
| 99 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSNLAKKA |
| | LEIILRAAAALANLPDPESRKEADKAADKVRREQPGSELAVVAAIISAVARMGVKME |
| | LHPSGNEVKVVIKGLHIKQQRQLYRDVREAAKKAGVEVEIEVEGDTVTIVVRG |

name: DFB0; alt. name: 274B_53_0_101A; type: Connector bivalent

| | |
|---|---|
| 100 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRGGSHHWGSGSHHHHHH |
| 101 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRG |

name: DF202; alt. name: 274B_62_0_202Av2; type: Connector bivalent

| | |
|---|---|
| 102 | NTHFIVVHGTEEARQLAEEIVRLIAEALGTEVRFEKKDGLLSIEVKNLPEEAQRLIQ |
| | KLLQLAVRIAQAAKSGDNDQLRELAEDALRLAKEAEKLGDLGAAAVAARLAVEAAKQ |
| | AGDNDVLRKVAEQALRIAKEAEKQGLVDVAVEAARVAVEAAKQAGDNDVLRKVAEQA |
| | LRIAKEAEKQGNVEVAVKAARVAVEAAKQAGDNDVLRKVAEQALRIVREALKQGNKE |
| | VAKKALEVAIEAANQAGDQKLLAEILLLAIEVLVVEMGVTMETHKSGNKVKVVIKGL |
| | HESQQETLRKLVHELLRKLGVVAVTQKHGDTVTIYVTEGGSHHWGSGSHHHHHH |
| 103 | NTHFIVVHGTEEARQLAEEIVRLIAEALGTEVRFEKKDGLLSIEVKNLPEEAQRLIQ |
| | KLLQLAVRIAQAAKSGDNDQLRELAEDALRLAKEAEKLGDLGAAAVAARLAVEAAKQ |
| | AGDNDVLRKVAEQALRIAKEAEKQGLVDVAVEAARVAVEAAKQAGDNDVLRKVAEQA |
| | LRIAKEAEKQGNVEVAVKAARVAVEAAKQAGDNDVLRKVAEQALRIVREALKQGNKE |
| | VAKKALEVAIEAANQAGDQKLLAEILLLAIEVLVVEMGVTMETHKSGNKVKVVIKGL |
| | HESQQETLRKLVHELLRKLGVVAVTQKHGDTVTIYVTE |

name: DF206; alt. name: 274B_62_0_206Bv2; type: Connector bivalent

| | |
|---|---|
| 104 | NTHFIVVHGTEEARQLAEEIVRLIAEALGTEVRFEKKDGLLSIEVKNLPEEAQRLIQ |
| | KLLQLAVRIAAAAESGDNDQLRELAEDALRLAEEAEKLGDLGAAAVAARLAVEAAKQ |
| | AGDNDVLREVAEQALEIAKEAEKQGLVDVAVEAARVAVEAAKQAGDNDVLRKVAEQA |
| | LRIAKEAEKQGNVEVAVKAARVAVEAAKQAGDNDVLRKVAEQALRIAKEAEKQGNVE |
| | VAALALVVAVNAAQAAGDQDLLRKIAEQAERLAKLAEKQGRRDVALLASIIALVAKM |
| | GVPMEVHPSGNEVKVVIKGLHKSQQEQLLKEVLKAANKLGVNVHISFRGDTVTIRVR |
| | GGGSHHWGSGSHHHHHH |
| 105 | NTHFIVVHGTEEARQLAEEIVRLIAEALGTEVRFEKKDGLLSIEVKNLPEEAQRLIQ |
| | KLLQLAVRIAAAAESGDNDQLRELAEDALRLAEEAEKLGDLGAAAVAARLAVEAAKQ |
| | AGDNDVLREVAEQALEIAKEAEKQGLVDVAVEAARVAVEAAKQAGDNDVLRKVAEQA |
| | LRIAKEAEKQGNVEVAVKAARVAVEAAKQAGDNDVLRKVAEQALRIAKEAEKQGNVE |
| | VAALALVVAVNAAQAAGDQDLLRKIAEQAERLAKLAEKQGRRDVALLASIIALVAKM |
| | GVPMEVHPSGNEVKVVIKGLHKSQQEQLLKEVLKAANKLGVNVHISFRGDTVTIRVR |
| | G |

name: DFX; alt. name: 274B_d62_−1_317A_d71; type: Connector bivalent

| | |
|---|---|
| 106 | HHHHHHGSGSNTHFIVVHGTEEARQLAEEIVRLIAKALGTEVRFEKKDGLLSIEVKN |
| | LPEEAQRLIQKLLQLAVRIAAAAKSGDNDVLRKLAEDALRLAKEAEKLGDLGAAAVA |
| | ARLAVEAAKQAGDNDVLRKVAEQALRIAKEAEKQGLVDVAVEAARVAVEAAKQAGDN |
| | DVLRKVAEQALRIAKEAEKQGNVEVAIKALEVAAEAAAQAGDKDVLKKILEQLERLA |
| | ELAKKQGNKELAIKIFELFIKVIVALMGVRMLSHKGGNAVIVVIEGLHPSQAEQLLR |
| | LVHRIAKKAGVTVHLVFTGDIVVIMVVVGASEEEQEDMHRLVREIAEALHFAKSFGA |
| | DEKALELLLKALLALLELVVASKEGDEEEFRKLAEKALELAKQLVELAKKLGIAALV |
| | LLAARIALKVAELAAKNGDKEVFKKAAESALEVAKRLVEVASKEGDAELVLEAAKVA |
| | LRVAELAAKNGDKEVFKKAAESALEVAKRLVEVASKEGDAELVEEAAKVAEEVRKLA |
| | KKQGDEEVYEKARETAREVKEELKRVREEKGDGS |
| 107 | NTHFIVVHGTEEARQLAEEIVRLIAKALGTEVRFEKKDGLLSIEVKNLPEEAQRLIQ |
| | KLLQLAVRIAAAAKSGDNDVLRKLAEDALRLAKEAEKLGDLGAAAVAARLAVEAAKQ |
| | AGDNDVLRKVAEQALRIAKEAEKQGLVDVAVEAARVAVEAAKQAGDNDVLRKVAEQA |
| | LRIAKEAEKQGNVEVAIKALEVAAEAAAQAGDKDVLKKILEQLERLAELAKKQGNKE |
| | LAIKIFELFIKVIVALMGVRMLSHKGGNAVIVVIEGLHPSQAEQLLRLVHRIAKKAG |
| | VTVHLVFTGDIVVIMVVVGASEEEQEDMHRLVREIAEALHFAKSFGADEKALELLLK |
| | ALLALLELVVASKEGDEEEFRKLAEKALELAKQLVELAKKLGIAALVLLAARIALKV |
| | ELAAKNGDKEVFKKAAESALEVAKRLVEVASKEGDAELVLEAAKVALRVAELAAKN |
| | GDKEVFKKAAESALEVAKRLVEVASKEGDAELVEEAAKVAEEVRKLAKKQGDEEVYE |
| | KARETAREVKEELKRVREEKGD |

name: DF275A-1; alt. name: 275A_d54_−1_206A; type: Connector bivalent

| | |
|---|---|
| 108 | HHHHHHGSGSGRQEKVLKSIEETVRKMGVEMLTFRAGKAVIVVIRGLHPEQAKQLLR |
| | DVSQTAHKQGVTVTLTFHGDVVFILVLVGASEEQQRAMQLLIQALARIIHEAKRRGV |
| | SEEQLKRMIEAAARLIEVLLKALEAAREGNTDEVREQLQRALEIVREIGLTAAVRLA |
| | LLVVEAVATLAAKRGNTDAVREALEVALEIARESGTTEAVKLALEVVARVAIEAARR |
| | GNTDAVREALEVALEIARESGTTEAVKLALEVVARVAIEAARRGNTDAVREALAVAV |
| | KIALKSGTEEAFRLAKEVIKRVSDEAKKQGNEDAVKEAESFDAAAELILSLLKLFRE |
| | LHERGTEIVVEVHINGRKTEIEVQGIDKRLLQIILEVIIEEIAREGPDKVEVNVHSG |
| | GQTWTFRYGGS |
| 109 | GRQEKVLKSIEETVRKMGVEMLTFRAGKAVIVVIRGLHPEQAKQLLRDVSQTAHKQG |
| | VTVTLTFHGDVVFILVLVGASEEQQRAMQLLIQALARIIHEAKRRGVSEEQLKRMIE |
| | AAARLIEVLLKALEAAREGNTDEVREQLQRALEIVREIGLTAAVRLALLVVEAVATL |
| | AAKRGNTDAVREALEVALEIARESGTTEAVKLALEVVARVAIEAARRGNTDAVREAL |
| | EVALEIARESGTTEAVKLALEVVARVAIEAARRGNTDAVREALAVAVKIALKSGTEE |
| | AFRLAKEVIKRVSDEAKKQGNEDAVKEAESFDAAAELILSLLKLFRELHERGTEIVV |
| | EVHINGRKTEIEVQGIDKRLLQIILEVIIEE IAREGPDKVEVNVHSGGQTWTFRYG |

name: DF284B; alt. name: 284B_04_−1_101B; type: Connector bivalent

| | |
|---|---|
| 110 | HHHHHHGSGSPHQFYVYQIDEHVAQLIEKFVRDISRREGTEVRFEKRDGQLEIEVKN |
| | LHEAQAIAIGIYIMILLLHQSGTSEDEIAEEIAKLIKGFIEHLKREGSSYEVICEAV |
| | AAAVAAIVKALKGCGTSEDEIAEIVARVISEVIRTLKESGSSYEVICECVARIVAEI |
| | VQALKESGTSEDEIAEIVARVISEVIRTLKESGSSYEVICECVARIVAEIVEALKRS |
| | GTSEEEIAEIVARVIQEVIRTLKESGSSYEVIRECLRRILEEVIEALKRSGVDSSEI |
| | VLIIIKIAVAVMGVTMEEHRSGNEVKVVIKGLHESQQEELLELVLRAAELAGVRVRI |
| | RFKGDTVTIVVRGGS |
| 111 | PHQFYVYQIDEHVAQLIEKFVRDISRREGTEVRFEKRDGQLEIEVKNLHEAQAIAIG |
| | IYIMILLLHQSGTSEDEIAEEIAKLIKGFIEHLKREGSSYEVICEAVAAAVAAIVKA |
| | LKGCGTSEDEIAEIVARVISEVIRTLKESGSSYEVICECVARIVAEIVQALKESGTS |
| | EDEIAEIVARVISEVIRTLKESGSSYEVICECVARIVAEIVEALKRSGTSEEEIAEI |
| | VARVIQEVIRTLKESGSSYEVIRECLRRILEEVIEALKRSGVDSSEIVLIIIKIAVA |
| | VMGVTMEEHRSGNEVKVVIKGLHESQQEELLELVLRAAELAGVRVRIRFKGDTVTIV |
| | VRG |

name: DF321; alt. name: 321B_53_0_101Av2; type: Connector bivalent

| | |
|---|---|
| 112 | TVTFDITNIDDKSTKLIATAVIHIAGREGTTVHFQGHDGQLEIEVKNLHEKWKRLIE |
| | MLIEAARRAQDPDPESLKEAVRIAEELVRLHPGNMLAEAALKVILTAAEELAKLPDP |
| | EALKEAVKAAEKVVREQPGSNLAKKALEIILRAAEELAKLPDPEALKEAVKAAEKVV |
| | REQPGSNLAKKALEIILRAAEELAKLPDPEALKEAVKAAEKVVREQPGSNLAKKALE |
| | IILRAAAALANLPDPESRKEADKAADKVEREQPGSELAVVAAIISAVARMGVTMELH |
| | PSGNEVKVVIKGLHIKQQRQLYRDVREAAKKAGVEVEIEVEGDTVTIVVRGGGSHHW |
| | GSGSHHHHHH |
| 113 | TVTFDITNIDDKSTKLIATAVIHIAGREGTTVHFQGHDGQLEIEVKNLHEKWKRLIE |
| | MLIEAARRAQDPDPESLKEAVRIAEELVRLHPGNMLAEAALKVILTAAEELAKLPDP |
| | EALKEAVKAAEKVVREQPGSNLAKKALEIILRAAEELAKLPDPEALKEAVKAAEKVV |
| | REQPGSNLAKKALEIILRAAEELAKLPDPEALKEAVKAAEKVVREQPGSNLAKKALE |
| | IILRAAAALANLPDPESRKEADKAADKVEREQPGSELAVVAAIISAVARMGVTMELH |
| | PSGNEVKVVIKGLHIKQQRQLYRDVREAAKKAGVEVEIEVEGDTVTIVVRG |

name: RingA; alt. name: 29B_53_101A; type: Connector bivalent

| | |
|---|---|
| 114 | GSSIFLLSNVSEDAAQLAEELVREISKKEGTEVRFEKDDGELTIEVKNLSEERLREI |
| | AKALQLIVDVANAERVVRERPGSNLAKKALEIILRAAEELAKLDLKASLKAAVRAAL |
| | KVVAEQPGSNLAKKALEIIQRAAEELAKLPDPEAQKEAQLAAELVRAAELAKSPDPE |
| | DLKEAVRLAEEVVRERPGSNLAKAALAIILRAAEELAKLPDPEALKEAVKAAEKVVR |
| | EQPGSNLAKKALEIILRAAAALANLPDPESRKEADKAADKVRREQPGSELAVVAAII |
| | SAVARMGVKMELHPSGNEVKVVIKGLHIKQQRQLYRDVREAAKKAGVEVEIEVEGDT |
| | VTIVVRGGGSWGLEHHHHHH |
| 115 | TWQWVLINISEEARQLIEKAVRAISKKEGTEVHFEKDDGVLHIRVKNLHEKRAREIH |
| | KVAKLILEVAAAERIVRERPGSNLAKKALEIILRAAEELAKADVDAALEAAVRAAEK |
| | VVREQPGSNLAKKAQEIILRAAEELAKLPDPEAQKEAAKAIARRVAAKVERLKRSGT |
| | SEDEIAEEVAREISEVIRTLKESGSSYEVICECVARIVAEIVEALKRSGTSEDEIAE |
| | IVARVISEVIRTLKESGSSYEVICECVARIVAEIVEALKRSGTSEEEIAEIVARVIQ |
| | EVIRTLKESGSSYEVIRECLRRILEEVIEALKRSGVDSSEIVLIIIKIAVAVMGVTM |
| | EEHRSGNEVKVVIKGLHESQQEELLELVLRAAELAGVRVRIRFKGDTVTIVVRG |

name: RingB; alt. name: 29A_53_4_101B; type: Connector bivalent

| | |
|---|---|
| 116 | GTWQWVLINISEEARQLIEKAVRAISKKEGTEVHFEKDDGVLHIRVKNLHEKRAREI |
| | HKVAKLILEVAAAERIVRERPGSNLAKKALEIILRAAEELAKADVDAALEAAVRAAE |
| | KVVREQPGSNLAKKAMEIILRAAEELAKLPDPEAQKEAAKAIARRVAAKVEELKRSG |
| | TSEDEIAEEVAREISEVIRTLKESGSSYEVICECVARIVAEIVEALKRSGTSEDEIA |
| | EIVARVISEVIRTLKESGSSYEVICECVARIVAEIVEALKRSGTSEEEIAEIVARVI |
| | QEVIRTLKESGSSYEVIRECLRRILEEVIEALKRSGVDSSEIVLIIIKIAVAVMGVT |
| | MEEHRSGNEVKVVIKGLHESQQEELLELVLRAAELAGVRVRIRFKGDTVTIVVRGGG |
| | SLEHHHHHH |
| 117 | TWQWVLINISEEARQLIEKAVRAISKKEGTEVHFEKDDGVLHIRVKNLHEKRAREIH |
| | KVAKLILEVAAAERIVRERPGSNLAKKALEIILRAAEELAKADVDAALEAAVRAAEK |
| | VVREQPGSNLAKKAQEIILRAAEELAKLPDPEAQKEAAKAIARRVAAKVERLKRSGT |
| | SEDEIAEEVAREISEVIRTLKESGSSYEVICECVARIVAEIVEALKRSGTSEDEIAE |
| | IVARVISEVIRTLKESGSSYEVICECVARIVAEIVEALKRSGTSEEEIAEIVARVIQ |
| | EVIRTLKESGSSYEVIRECLRRILEEVIEALKRSGVDSSEIVLIIIKIAVAVMGVTM |
| | EEHRSGNEVKVVIKGLHESQQEELLELVLRAAELAGVRVRIRFKGDTVTIVVRG |

name: DFA-GFP; alt. name: DF530A-GFP; type: Connector bivalent

| | |
|---|---|
| 118 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRGGSGSGSGSSKGEELFTGV |
| | VPILVELDGDVNGHKFSVRGEGEGDATNGKLTLKFICTTGKLPVPWPTLVTTLTYGV |
| | QCFARYPDHMKQHDFFKSAMPEGYVQERTISFKDDGTYKTRAEVKFEGDTLVNRIEL |
| | KGIDFKEDGNILGHKLEYNFNSHNVYITADKQKNGIKANFKIRHNVEDGSVQLADHY |
| | QQNTPIGDGPVLLPDNHYLSTQSVLSKDPNEKRDHMVLLEFVTAAGITHGMDELYK |
| 119 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRG |

name: GFP-DFA; alt. name: GFP-DF530A; type: Connector bivalent

| | |
|---|---|
| 120 | MSKGEELFTGVVPILVELDGDVNGHKFSVRGEGEGDATNGKLTLKFICTTGKLPVPW |
| | PTLVTTLTYGVQCFARYPDHMKQHDFFKSAMPEGYVQERTISFKDDGTYKTRAEVKF |
| | EGDTLVNRIELKGIDFKEDGNILGHKLEYNFNSHNVYITADKQKNGIKANFKIRHNV |
| | EDGSVQLADHYQQNTPIGDGPVLLPDNHYLSTQSVLSKDPNEKRDHMVLLEFVTAAG |
| | ITHGMDELYKGSGSGSGSTTNFHLINGSEEARQLIEKAVRAISKKEGTEVHFEKSDG |
| | TLEIRVKNLHEKREREIKKVIELILEVAAAERIVRERPGSNLAKKALEIILRAAEEL |
| | AKADVDAALEAAVRAAEKVVREQPGSNLAKKALEIILRAAEELAKLPDPEALKEAVK |
| | AAEKVVREQPGSNLAKKALEIILRAAEELAKLPDPEALKEAVKAAEKVVREQPGSNL |
| | AKKALEIILRAAAALANLPDPESRKEADKAADKVRREQPGSELAVVAAIISAVARMG |
| | VKMELHPSGNEVKVVIKGLHIKQQRQLYRDVREAAKKAGVEVEIEVEGDTVTIVVRG |
| | LEHHHHHH |
| 121 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSNLAKKA |
| | LEIILRAAEELAKLPDPEALKEAVKAAEKVVREQPGSNLAKKALEIILRAAAALANL |
| | PDPESRKEADKAADKVRREQPGSELAVVAAIISAVARMGVKMELHPSGNEVKVVIKG |
| | LHIKQQRQLYRDVREAAKKAGVEVEIEVEGDTVTIVVRG |

name: 101A10; alt. name: LHD101_A_DHR10_N; type: single fusion
monovalent

| | |
|---|---|
| 122 | HHHHHHGSGLNDIFEAQKIEWHEAENLYFQSGSSSEKEELRERLVKIVVENAKRKGD |
| | DTEEAREAAREAFELVREAAERAGIDSSEVLELAIRLIKEVVENAQREGYDISEAAR |
| | AAAEAFKRVAEAAKRAGITSSEVLELAIILIKLVVELAQRKGYDISEAARAAAELFK |
| | RLAEALKRAGKTSERALALLILLLAIEILVRDMGVTMETHPSGNEVKVVIKGLHIKQ |
| | QRQLYRLVREAAKLLGVEVEIEVEGDTVTIVVRGGS |
| 123 | SSEKEELRERLVKIVVENAKRKGDDTEEAREAAREAFELVREAAERAGIDSSEVLEL |
| | AIRLIKEVVENAQREGYDISEAARAAAEAFKRVAEAAKRAGITSSEVLELAIILIKL |
| | VVELAQRKGYDISEAARAAAELFKRLAEALKRAGKTSERALALLILLLAIEILVRDM |
| | GVTMETHPSGNEVKVVIKGLHIKQQRQLYRLVREAAKLLGVEVEIEVEGDTVTIVVR |
| | G |

name: 101A21; alt. name: LHD101_A_DHR21_N; type: single fusion
monovalent

| | |
|---|---|
| 124 | HHHHHHGSGLNDIFEAQKIEWHEAENLYFQSGSSEKEKVEELAQRIREQLPDTELAR |
| | EAQELADEARKSDDSEALKVVYLALRIVQQLPDTELAREALELAKEAVKSTDSEKLK |
| | VVYLALRVVQQLPDTEEARKALEIAKEAVKADAQILLAIARAVLKMGVEMEVHPSGN |
| | EVKVVIKGLHIKQQRQLYRDVREAAKKAGVEVEIEVEGDTVTIVVRE |
| 125 | SEKEKVEELAQRIREQLPDTELAREAQELADEARKSDDSEALKVVYLALRIVQQLPD |
| | TELAREALELAKEAVKSTDSEKLKVVYLALRVVQQLPDTEEARKALEIAKEAVKADA |
| | QILLAIARAVLKMGVEMEVHPSGNEVKVVIKGLHIKQQRQLYRDVREAAKKAGVEVE |
| | IEVEGDTVTIVVRE |

name: 101A52; alt. name: LHD101_A_DHR52_N; type: single fusion
monovalent

| | |
|---|---|
| 126 | HHHHHHGSGLNDIFEAQKIEWHEAENLYFQSGSCEDRKEKIRELERKARENTGSDEA |
| | RQAVKEIARIAKEALEEGCCDTAKEAIQRLEDLARDYSGSDVASLAVKAIAKIAETA |
| | LRNGCCDTAKEAIQRLEDLARDYSGSDVASLAVEAILRIALIALANGCEETAEEARK |
| | RLRELAEDYKGSEVAKLAESAERLIEILKIIAKTVRKMGVTMDVRPSGTEVEVVIKG |
| | LHIKQQRQLYRDVREAAKKLGVEVEIEVEGDTVTIVVRGGS |
| 127 | CEDRKEKIRELERKARENTGSDEARQAVKEIARIAKEALEEGCCDTAKEAIQRLEDL |
| | ARDYSGSDVASLAVKAIAKIAETALRNGCCDTAKEAIQRLEDLARDYSGSDVASLAV |
| | EAILRIALIALANGCEETAEEARKRLRELAEDYKGSEVAKLAESAERLIEILKIIAK |
| | TVRKMGVTMDVRPSGTEVEVVIKGLHIKQQRQLYRDVREAAKKLGVEVEIEVEGDTV |
| | TIVVRG |

name: 101A53; alt. name: LHD101_A_DHR53_N; type: single fusion
monovalent

| | |
|---|---|
| 128 | HHHHHHGSGLNDIFEAQKIEWHEAENLYFQSGSNDEKEKLKELLKRAEELAKSPDPE |
| | DLKEAVRLAEEVVRERPGSNLAKKALEIILRAAEELAKLPDPEALKEAVKAAEKVVR |
| | EQPGSNLAKKALEIILRAAAALANLPDPESRKEADKAADKVRREQPGSELAVVAAII |
| | SAVARMGVKMELHPSGNEVKVVIKGLHIKQQRQLYRDVREAAKKAGVEVEIEVEGDT |
| | VTIVVRG |
| 129 | NDEKEKLKELLKRAEELAKSPDPEDLKEAVRLAEEVVRERPGSNLAKKALEIILRAA |
| | EELAKLPDPEALKEAVKAAEKVVREQPGSNLAKKALEIILRAAAALANLPDPESRKE |
| | ADKAADKVRREQPGSELAVVAAIISAVARMGVKMELHPSGNEVKVVIKGLHIKQQRQ |
| | LYRDVREAAKKAGVEVEIEVEGDTVTIVVRG |

name: 101B4; alt. name: LHD101_B_DHR04_N; type: single fusion
monovalent

| | |
|---|---|
| 130 | HHHHHHGSGLNDIFEAQKIEWHEAENLYFQSGSYEDECEEKARRVAEKVERLKRSGT |
| | SEDEIAEEVAREISEVIRTLKESGSSYEVICECVARIVAEIVEALKRSGTSEDEIAE |
| | IVARVISEVIRTLKESGSSYEVICECVARIVAEIVEALKRSGTSEEEIAEIVARVIQ |
| | EVIRTLKESGSSYEVIRECLRRILEEVIEALKRSGVDSSEIVLIIIKIAVAVMGVTM |
| | EEHRSGNEVKVVIKGLHESQQEELLELVLRAAELAGVRVRIRFKGDTVTIVVRG |
| 131 | YEDECEEKARRVAEKVERLKRSGTSEDEIAEEVAREISEVIRTLKESGSSYEVICEC |
| | VARIVAEIVEALKRSGTSEDEIAEIVARVISEVIRTLKESGSSYEVICECVARIVAE |
| | IVEALKRSGTSEEEIAEIVARVIQEVIRTLKESGSSYEVIRECLRRILEEVIEALKR |
| | SGVDSSEIVLIIIKIAVAVMGVTMEEHRSGNEVKVVIKGLHESQQEELLELVLRAAE |
| | LAGVRVRIRFKGDTVTIVVRG |

name: 101B8; alt. name: LHD101_B_DHR08_N; type: single fusion
monovalent

| | |
|---|---|
| 132 | HHHHHHGSGLNDIFEAQKIEWHEAENLYFQSGSDEMKKVMEALKKAVELAKKNNDDE |
| | VAREIERAAKEIVEALRENNSDEMAKVMLALAKAVLLAAKNNDDEVAREIARAAAEI |
| | VEALRENNLEVMALVARLLAEAVLLAAKNNDDEVAREIAREAAEIVEKLRENNDATM |
| | AVRAAIRAAVIRMGVTMEEHRSGNEVKVVIKGLHESQQEELLEIVLRAAELAGVRVR |
| | IRFKGDTVTIVVRG |
| 133 | DEMKKVMEALKKAVELAKKNNDDEVAREIERAAKEIVEALRENNSDEMAKVMLALAK |
| | AVLLAAKNNDDEVAREIARAAAEIVEALRENNLEVMALVARLLAEAVLLAAKNNDDE |
| | VAREIAREAAEIVEKLRENNDATMAVRAAIRAAVIRMGVTMEEHRSGNEVKVVIKGL |
| | HESQQEELLEIVLRAAELAGVRVRIRFKGDTVTIVVRG |

name: 101B14; alt. name: LHD101_B_DHR14a_N; type: single fusion
monovalent

| | |
|---|---|
| 134 | HHHHHHGSGLNDIFEAQKIEWHEAENLYFQSGSSEEVNERVKQLAEKAKEATDKEEV |
| | IEIVKELAELAKQSTDSELVNEIVKQLAEVAKEATDKELVIYIVKILAELAKQSTDS |
| | ELVNEIVKQLAEVAKEATDKELVIYIVDILLKLAEQADDDELVEEIRKQLEEVAKEA |
| | TDKELVEIIKAVIVLLVIISVVARMGVTMEIHKSGREVKVVIKGLHESQQEQLLEAV |
| | LRAAEEAGVRVRIRFKGDTVTIVVRG |
| 135 | SEEVNERVKQLAEKAKEATDKEEVIEIVKELAELAKQSTDSELVNEIVKQLAEVAKE |
| | ATDKELVIYIVKILAELAKQSTDSELVNEIVKQLAEVAKEATDKELVIYIVDILLKL |
| | AEQADDDELVEEIRKQLEEVAKEATDKELVEIIKAVIVLLVIISVVARMGVTMEIHK |
| | SGREVKVVIKGLHESQQEQLLEAVLRAAEEAGVRVRIRFKGDTVTIVVRG |

name: 101B62; alt. name: LHD101_B_DHR62_N; type: single fusion
monovalent

| | |
|---|---|
| 136 | HHHHHHGSGLNDIFEAQKIEWHEAENLYFQSGSNDEKRKRAEKALQRAQEAEKKGDV |
| | EEAVRAAQEAVRAAKESGDNDVLRKVAEQALRIAKEAEKQGNVEVAVKAARVAVEAA |
| | KQAGDNDVLRKVAEQALRIAKEALKQGNVDVAAKAAQVAEEAAKQAGDQDVLRKVKE |
| | QIEIVLAAIELTVRKMGVTMETHRSGREVKVVIKGLHESQQEQLLEDVLRIAELAG |
| | VRVRIRFKGDTVTIVVRG |
| 137 | NDEKRKRAEKALQRAQEAEKKGDVEEAVRAAQEAVRAAKESGDNDVLRKVAEQALRI |
| | AKEAEKQGNVEVAVKAARVAVEAAKQAGDNDVLRKVAEQALRIAKEALKQGNVDVAA |
| | KAAQVAEEAAKQAGDQDVLRKVKEVQIEIVLAAIELTVRKMGVTMETHRSGREVKVV |
| | IKGLHESQQEQLLEDVLRIAELAGVRVRIRFKGDTVTIVVRG |

name: 101B82; alt. name: LHD101_B_DHR82_N; type: single fusion
monovalent

| | |
|---|---|
| 138 | HHHHHHGSGLNDIFEAQKIEWHEAENLYFQSGSDEEVQEAVERAEELREEAEELIKK |
| | ARKTGDAELLRKALEALEEAVRAVEEAIKRNPDNDEAVETAVRLARELKKVAEELQE |
| | RAKKTGDAELLKLALRALEVAVRAVELAIKSNPDNDEAVETAVRLARELAKVAEELI |
| | ERAKKTGDKELLKLAKRALEVAMRAVSLALKSNPDNEEARRVAAELVLLVIRAAVIE |
| | MGVTMEEHRSGNRVKVVIKGLHESQQEQLLEDVLRAAEIAGVRVRIRFKGDTVTIVV |
| | EG |
| 139 | DEEVQEAVERAEELREEAEELIKKARKTGDAELLRKALEALEEAVRAVEEAIKRNPD |
| | NDEAVETAVRLARELKKVAEELQERAKKTGDAELLKLALRALEVAVRAVELAIKSNP |
| | DNDEAVETAVRLARELAKVAEELIERAKKTGDKELLKLAKRALEVAMRAVSLALKSN |
| | PDNEEARRVAAELVLLVIRAAVIEMGVTMEEHRSGNRVKVVIKGLHESQQEQLLEDV |
| | LRAAEIAGVRVRIRFKGDTVTIVVEG |

name: 202A21; alt. name: LHD202_A_DHR21_N; type: single fusion
monovalent

| | |
|---|---|
| 140 | HHHHHHGSGLNDIFEAQKIEWHEAENLYFQSGSSEKEKVEELAQRIREQLPDTELAR |
| | EAQELADEARKSDDSEALKVVYLALRIVQQLPDTELAREALELAKEAVKSTDPAQLI |
| | VVQLALKIVQKLPDTEEARRALELAKEAVKSTNKAELVVIAIELLVLLMGVTMEVHK |
| | SGNKVKVVIKGLHESQQEQLRKLVHEALRAAGVVAVTQKHGDTVTIYVTEGS |
| 141 | SEKEKVEELAQRIREQLPDTELAREAQELADEARKSDDSEALKVVYLALRIVQQLPD |
| | TELAREALELAKEAVKSTDPAQLIVVQLALKIVQKLPDTEEARRALELAKEAVKSTN |
| | KAELVVIAIELLVLLMGVTMEVHKSGNKVKVVIKGLHESQQEQLRKLVHEALRAAGV |
| | VAVTQKHGDTVTIYVTE |

name: 202A62; alt. name: LHD202_A_DHR62_N; type: single fusion
monovalent

| | |
|---|---|
| | Nter his-avi-tev |
| 142 | HHHHHHGSGLNDIFEAQKIEWHEAENLYFQSGSNDEKRKRAEKALQRAQEAEKKGDV |
| | EEAVRAAQEAVRAAKESGDNDVLRKVAEQALRIAKEAEKQGNVEVAVKAARVAVEAA |
| | KQAGDNDVLRKVAEQALRIVREALKQGNKEVAKKALEVAIEAANQAGDQKLLSKILQ |
| | LAIEVLVVEMGVTMETHKSGNKVKVVIKGLHESQQETLRKLVHELLRKLGVVAVTQK |
| | HGDTVTIYVTEGS |
| 143 | NDEKRKRAEKALQRAQEAEKKGDVEEAVRAAQEAVRAAKESGDNDVLRKVAEQALRI |
| | AKEAEKQGNVEVAVKAARVAVEAAKQAGDNDVLRKVAEQALRIVREALKQGNKEVAK |
| | KALEVAIEAANQAGDQKLLSKILQLAIEVLVVEMGVTMETHKSGNKVKVVIKGLHES |
| | QQETLRKLVHELLRKLGVVAVTQKHGDTVTIYVTE |

name: 202B57; alt. name: LHD202_B_DHR57_C; type: single fusion
monovalent

| | |
|---|---|
| 144 | GSSVEFHIVNISEKAAQIIERAVRAISKELGTEVRFEKRDGELTIEVKNLHERRLQE |
| | ILLLIEAVKLLLLALKAVKEDPSTDALRAVLEAVRFASEVAKRVENPEAVAVLAELV |
| | IELALEAVKEDPSTDALRAVLEAVRLASEVAKRVTDADKALKIAKLVIELALEAVKE |
| | DPSEEAKRAVEEAKRLAEEVSKRVTDPELSEKIRQLVKELEEEAQKEDPSGSHHWGS |
| | GLNDIFEAQKIEWHEGSHHHHHH |
| 145 | SVEFHIVNISEKAAQIIERAVRAISKELGTEVRFEKRDGELTIEVKNLHERRLQEIL |
| | LLIEAVKLLLLALKAVKEDPSTDALRAVLEAVRFASEVAKRVENPEAVAVLAELVIE |
| | LALEAVKEDPSTDALRAVLEAVRLASEVAKRVTDADKALKIAKLVIELALEAVKEDP |
| | SEEAKRAVEEAKRLAEEVSKRVTDPELSEKIRQLVKELEEEAQKEDPS |

name: 202B64; alt. name: LHD202_B_DHR64_C; type: single fusion
monovalent

| | |
|---|---|
| 146 | GSSVEFHIVNIDEDVAQLIELAVKLISKEEGTEVRFEKRDGELTIEVKNLHEKDLQL |
| | ILELIEALLLIARAIELLRQAKEKGSEEDLEKALRTAEESARRLKKVLEKAEKLGNL |
| | GVALAAVAGVVLVAELLLRIAKESGSEEALERALRVAEEAARLAKRVLELAEKLGDA |
| | EAALLAVELVVRVAELLLRIAKESGSEEALERALRVAEEAARLAKRVLELAEKQGDA |
| | EVARRAVELVKRVAELLERIARESGSEEAKERAERVREEARELQERVKELREREGDG |
| | SHHWGSGLNDIFEAQKIEWHEGSHHHHHH |
| 147 | SVEFHIVNIDEDVAQLIELAVKLISKEEGTEVRFEKRDGELTIEVKNLHEKDLQLIL |
| | ELIEALLLIARAIELLRQAKEKGSEEDLEKALRTAEESARRLKKVLEKAEKLGNLGV |
| | ALAAVAGVVLVAELLLRIAKESGSEEALERALRVAEEAARLAKRVLELAEKLGDAEA |
| | ALLAVELVVRVAELLLRIAKESGSEEALERALRVAEEAARLAKRVLELAEKQGDAEV |
| | ARRAVELVKRVAELLERIARESGSEEAKERAERVREEARELQERVKELREREGD |

name: 206A54; alt. name: LHD206_A_DHR54_N; type: single fusion
monovalent

| | |
|---|---|
| 148 | HHHHHHGSGLNDIFEAQKIEWHEAENLYFQSGSTEDERRELEKVARKAIEAAREGNT |
| | DEVREQLQRALEIARESGTTEAVKLALEVVARVAIEAARRGNTDAVREALEVALEIA |
| | RESGTTEAVKLALEVVARVAIEAARRGNTDAVREALAVAVKIALKSGTEEAFRLAKE |
| | VIKRVSDEAKKQGNEDAVKEAESFDAAAELILSLLKLFRELHERGTEIVVEVHINGR |
| | KTEIEVQGIDKRLLQIILEVIIEEIAREGPDKVEVNVHSGGQTWTFRYGGS |
| 149 | TEDERRELEKVARKAIEAAREGNTDEVREQLQRALEIARESGTTEAVKLALEVVARV |
| | AIEAARRGNTDAVREALEVALEIARESGTTEAVKLALEVVARVAIEAARRGNTDAVR |
| | EALAVAVKIALKSGTEEAFRLAKEVIKRVSDEAKKQGNEDAVKEAESFDAAAELILS |
| | LLKLFRELHERGTEIVVEVHINGRKTEIEVQGIDKRLLQIILEVIIEEIAREGPDKV |
| | EVNVHSGGQTWTFRYG |

name: 206B62-1; alt. name: LHD206_B_DHR62_N1; type: single fusion
monovalent

| | |
|---|---|
| 150 | HHHHHHGSGLNDIFEAQKIEWHEAENLYFQSGSNDEKRKRAEKALQRAQEAEKKGDV |
| | EEAVRAAQEAVRAAKESGDNDVLRKVAEQALRIAKEAEKQGNVEVAVKAARVAVEAA |
| | KQAGDNDVLRKVAEQALRIAKEAEKQGNVEVAALALVVATNAAQAAGDQDLLRKIAE |
| | QAERLAKLAKKQGRRDVALLALIIALVSKMGVPMEVHPSGKEVKVVIKGLHKSQQEQ |
| | LLKLVLKAANKLGVNVHISFRGDTVTIRVRGGS |
| 151 | NDEKRKRAEKALQRAQEAEKKGDVEEAVRAAQEAVRAAKESGDNDVLRKVAEQALRI |
| | AKEAEKQGNVEVAVKAARVAVEAAKQAGDNDVLRKVAEQALRIAKEAEKQGNVEVAA |
| | LALVVATNAAQAAGDQDLLRKIAEQAERLAKLAKKQGRRDVALLALIIALVSKMGVP |
| | MEVHPSGKEVKVVIKGLHKSQQEQLLKLVLKAANKLGVNVHISFRGDTVTIRVRG |

name: 206B62-2; alt. name: LHD206_B_DHR62_N2; type: single fusion
monovalent

| | |
|---|---|
| 152 | HHHHHHGSGLNDIFEAQKIEWHEAENLYFQSGSNDEKRKRAEKALQRAQEAEKKGDV |
| | EEAVRAAQEAVRAAKESGDNDVLRKVAEQALRIAKEAEKQGNVEVAVKAARVAVEAA |
| | KQAGDNDVLRKVAEQALRIAKEAEKQGNVPVAVKALLVALNAAVAAGDQDVLRKISE |
| | QAERARKLAEKQGDKLLAFVLALISLVAQMGVPMEIHPSGNEVKVVIKGLHKSQQEQ |
| | LLKLVLKLANKLGVNVHISFRGDTVTIRVRGGS |
| 153 | NDEKRKRAEKALQRAQEAEKKGDVEEAVRAAQEAVRAAKESGDNDVLRKVAEQALRI |
| | AKEAEKQGNVEVAVKAARVAVEAAKQAGDNDVLRKVAEQALRIAKEAEKQGNVPVAV |
| | KALLVALNAAVAAGDQDVLRKISEQAERARKLAEKQGDKLLAFVLALISLVAQMGVP |
| | MEIHPSGNEVKVVIKGLHKSQQEQLLKLVLKLANKLGVNVHISFRGDTVTIRVRG |

name: 274A64; alt. name: LHD274_A_DHR64_C; type: single fusion
monovalent

| | |
|---|---|
| 154 | GSTTNFHLINGSEEARQLIQKAVEAISKKEGTEVHFEKSDGTLEIRVKNLHPRQEDL |
| | IKKFIEALLLALVAKGELEQAEKEGDAEVALRAVEKVVRVAELLLRLAKEAGSEEAL |
| | KAALEIAEQAARLAKRVLELAEKQGDAEVALRAVELVVRVAELLLRIAKESGSEEAL |
| | ERALRVAEEAARLAKRVLELAEKQGDAEVARRAVELVKRVAELLERIARESGSEEAK |
| | ERAERVREEARELQERVKELREREGDGSHHWGSGLNDIFEAQKIEWHEGSHHHHHH |
| 155 | TTNFHLINGSEEARQLIQKAVEAISKKEGTEVHFEKSDGTLEIRVKNLHPRQEDLIK |
| | KFIEALLLALVAKGELEQAEKEGDAEVALRAVEKVVRVAELLLRLAKEAGSEEALKA |
| | ALEIAEQAARLAKRVLELAEKQGDAEVALRAVELVVRVAELLLRIAKESGSEEALER |
| | ALRVAEEAARLAKRVLELAEKQGDAEVARRAVELVKRVAELLERIARESGSEEAKER |
| | AERVREEARELQERVKELREREGD |

name: 274A76; alt. name: LHD274_A_DHR76_C; type: single fusion
monovalent

| | |
|---|---|
| 156 | GSTTNFHLINGSEEARQVIEEIVEIIARLAGTEVHFEKSDGTLEIRVKNLHEELERL |
| | IKELIELALLLQLAKKEAIEEAKKQGNPELVEWVARAAEVVKEVLRVAAEAAGAGNP |
| | DLAKAAAELARAVIEAIEEAVKQGNAELVEWVARAAKVAAEVIKVAIQAEKEGNRDL |
| | FRAALELVRAVIEAIEEAVKQGNAELVERVARLAKKAAELIKRAIRAEKEGNRDERR |
| | EALERVREVIERIEELVRQGNGSHHWGSGLNDIFEAQKIEWHEGSHHHHHH |
| 157 | TTNFHLINGSEEARQVIEEIVEIIARLAGTEVHFEKSDGTLEIRVKNLHEELERLIK |
| | ELIELALLLQLAKKEAIEEAKKQGNPELVEWVARAAEVVKEVLRVAAEAAGAGNPDL |
| | AKAAAELARAVIEAIEEAVKQGNAELVEWVARAAKVAAEVIKVAIQAEKEGNRDLER |
| | AALELVRAVIEAIEEAVKQGNAELVERVARLAKKAAELIKRAIRAEKEGNRDERREA |
| | LERVREVIERIEELVRQGN |

name: 274B62; alt. name: LHD274 B DHR62 C; type: single fusion
monovalent

| | |
|---|---|
| 158 | GSNTHFIVVHGTEEARQLAEEIVRLIAKALGTEVRFEKKDGLLSIEVKNLPEEAQRL |
| | IQKLLQLAVRIAAAAKSGDNDVLRKLAEDALRLAKEAEKLGDLGAAAVAARLAVEAA |
| | KQAGDNDVLRKVAEQALRIAKEAEKQGLVDVAVEAARVAVEAAKQAGDQDVLRKVSE |
| | QAERISKEAKKQGNSEVSEEARKVADEAKKQTGDGSHHWGSGLNDIFEAQKIEWHEG |
| | SHHHHHH |
| 159 | NTHFIVVHGTEEARQLAEEIVRLIAKALGTEVRFEKKDGLLSIEVKNLPEEAQRLIQ |
| | KLLQLAVRIAAAAKSGDNDVLRKLAEDALRLAKEAEKLGDLGAAAVAARLAVEAAKQ |
| | AGDNDVLRKVAEQALRIAKEAEKQGLVDVAVEAARVAVEAAKQAGDQDVLRKVSEQA |
| | ERISKEAKKQGNSEVSEEARKVADEAKKQTGD |

name: 274B82; alt. name: LHD274 B DHR82 C; type: single fusion
monovalent

| | |
|---|---|
| 160 | GSNTHFIVVHGGEEARQLAETAVREISKKEGTEVRFEKKDGLLSIEVKNLSEELQRL |
| | IQELLQLLVRLAALLEAVRAVEEAIKRNPDNDEAVETAVRLARELKKVAELLQELAK |
| | KAGVPAILRGALLALEVAVRAVELAIKSNPDNDEAVETAVRLARELKKVAEELQERA |
| | KKTGDAELLKLALRALEVAVRAVELAIKSNPDNEEAVETAKRLAEELRKVAELLEER |
| | AKETGDPELQELAKRAKEVADRARELAKKSNPNNGSHHWGSGLNDIFEAQKIEWHEG |
| | SHHHHHH |
| 161 | NTHFIVVHGGEEARQLAETAVREISKKEGTEVRFEKKDGLLSIEVKNLSEELQRLIQ |
| | ELLQLLVRLAALLEAVRAVEEAIKRNPDNDEAVETAVRLARELKKVAELLQELAKKA |
| | GVPAILRGALLALEVAVRAVELAIKSNPDNDEAVETAVRLARELKKVAEELQERAKK |
| | TGDAELLKLALRALEVAVRAVELAIKSNPDNEEAVETAKRLAEELRKVAELLEERAK |
| | ETGDPELQELAKRAKEVADRARELAKKSNPNN |

name: 274A53; alt. name: LHD274A DHR53 stop; type: single_fusion
monovalent

| | |
|---|---|
| 162 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREESGSHHWGSGLNDIFEAQKIEWHEG |
| | SHHHHHH |
| 163 | TTNFHLINGSEEARQLIEKAVRAISKKEGTEVHFEKSDGTLEIRVKNLHEKREREIK |
| | KVIELILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREE |

name: 274B53; alt. name: LHD274B DHR53 stop; type: single fusion
monovalent

| | |
|---|---|
| 164 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREESGSHHWGSGLNDIFEAQKIEWHEG |
| | SHHHHHH |
| 165 | NTHFIVVHGSEDAAQLAEELVREISKKEGTEVRFEKKDGLLSIEVKNLSEERQREIQ |
| | KALQLVQDVANAERVVRERPGSNLAKKALEIILRAAEELAKLDLKASLKAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREE |

name: 284A82; alt. name: LHD284 A DHR82 N; type: single fusion
monovalent

| | |
|---|---|
| 166 | HHHHHHGSGLNDIFEAQKIEWHEAENLYFQSGSDEEVQEAVERAEELREEAEELIKK |
| | ARKTGDAELLRKALEALEEAVRAVEEAIKRNPDNDEAVETAVRLARELKKVAEELQE |
| | RAKKTGDAELLKLALRALEVAVRAVELAIKSNPDNDEAVETAVRLAKELLKVAILLA |
| | KRAQETGDKELEKLARRALEVAKRAVELAIKSNPDNKEARILKLLLELAELLIELAL |
| | RGTIIIVEVHINGERQTKYLILAPVEELLKHLERIEEKIKREGASEVEVKVTSGGTT |
| | WTFNIKGS |
| 167 | DEEVQEAVERAEELREEAEELIKKARKTGDAELLRKALEALEEAVRAVEEAIKRNPD |
| | NDEAVETAVRLARELKKVAEELQERAKKTGDAELLKLALRALEVAVRAVELAIKSNP |
| | DNDEAVETAVRLAKELLKVAILLAKRAQETGDKELEKLARRALEVAKRAVELAIKSN |
| | PDNKEARILKLLLELAELLIELALRGTIIIVEVHINGERQTKYLILAPVEELLKHLE |
| | RIEEKIKREGASEVEVKVTSGGTTWTFNIK |

name: 29A53; alt. name: LHD29 DHR53 AB_A_0008_0001.pdb; type:
single fusion monovalent

| | |
|---|---|
| 168 | TWQWVLINISEEARQLIEKAVRAISKKEGTEVHFEKDDGVLHIRVKNLHEKRAREIH |
| | KVAKLILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREERPGSSGSHHHHHH |
| 169 | TWQWVLINISEEARQLIEKAVRAISKKEGTEVHFEKDDGVLHIRVKNLHEKRAREIH |
| | KVAKLILEVAAAERIVRERPGSNLAKKALEIILRAAEELAKADVDAALEAAVRAAEK |
| | VVREQPGSNLAKKALEIILRAAEELAKLPDPEALKEAVKAAEKVVREQPGSELAKKA |
| | LEIIERAAEELKKSPDPEAQKEAKKAEQKVREERPGS |

name: 29B53; alt. name: LHD29 DHR53 AB_B_0005_0001.pdb; type:
single fusion monovalent

| | |
|---|---|
| 170 | SSIFLLSNVDESARQLAEELVREISKKEGTEVRFEKDDGFLTIEVKNLSEERLREIA |
| | RALQLIVDVANAERVVRERPGSNLAKKALEIILRAAEELAKLPLKASLKAAVIAAEL |
| | VVREQPGSNLAKKALEIILRAAEELAKLPDPEALKEAVKAAEKVVREQPGSELAKKA |
| | LEIIERAAEELKKSPDPEAQKEAKKAEQKVREERPGSSGSHHHHHH |
| 171 | SSIFLLSNVDESARQLAEELVREISKKEGTEVRFEKDDGFLTIEVKNLSEERLREIA |
| | RALQLIVDVANAERVVRERPGSNLAKKALEIILRAAEELAKLPLKASLKAAVIAAEL |
| | VVREQPGSNLAKKALEIILRAAEELAKLPDPEALKEAVKAAEKVVREQPGSELAKKA |
| | LEIIERAAEELKKSPDPEAQKEAKKAEQKVREERPGS |

name: 275B; alt. name: LHD275 B; type: single fusion monovalent

| | |
|---|---|
| 172 | GGSDVEWRYTNISEETQQKSAEFVLEIALRAGTGVTFTTRQGELQIQVHNLDELLAI |
| | AMLCYTLGLLLGDHRVQELAKRAVEAWERGDEERVKKLLIEALKRLVETAEEVVRER |
| | PGSNLAKLALEIILRAAEALARAEDPESLKEAVKAAEKVVREQPGSNLAKKALEIIL |
| | RAAEELAKLPDPEALKEAVKAAEKVVREQPGSELAKKALEIIERAAEELKKSPDPEA |
| | QKEAKKAEQKVREERPGSGGSGSHHWGSGSHHHHHH |
| 173 | DVEWRYTNISEETQQKSAEFVLEIALRAGTGVTFTTRQGELQIQVHNLDELLAIAML |
| | CYTLGLLLGDHRVQELAKRAVEAWERGDEERVKKLLIEALKRLVETAEEVVRERPGS |
| | NLAKLALEIILRAAEALARAEDPESLKEAVKAAEKVVREQPGSNLAKKALEIILRAA |
| | EELAKLPDPEALKEAVKAAEKVVREQPGSELAKKALEIIERAAEELKKSPDPEAQKE |
| | AKKAEQKVREERPGS |

name: 278B; alt. name: LHD278 B; type: single fusion monovalent

| | |
|---|---|
| 174 | GGSTVTFDITNIDWKSAELIMLAVYDIAQQEGTDVTFSFKEGELQITVKNLHEKWKR |
| | LIEMLIEACRRAQDPDPESLKEAVRIAEELVRLHPGNPLARAALKVILTAAEELAKL |
| | PDPEALKEAVKAAEKVVREQPGSNLAKKALEIILRAAEELAKLPDPEALKEAVKAAE |
| | KVVREQPGSELAKKALEIIERAAEELKKSPDPEAQKEAKKAEQKVREERPGSGGSGS |
| | HHWGSGSHHHHHH |
| 175 | TVTFDITNIDWKSAELIMLAVYDIAQQEGTDVTFSFKEGELQITVKNLHEKWKRLIE |
| | MLIEACRRAQDPDPESLKEAVRIAEELVRLHPGNPLARAALKVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |

name: 284B; alt. name: LHD284 B; type: single fusion monovalent

| | |
|---|---|
| 176 | GGSPHQFYVYQIDEHVAQLIEKFVRDISRREGTEVRFEKRDGQLEIEVKNLHEAQAI |
| | AIGIYIMILLLHQSGTSEDEIAEEIAKLIKGFIEHLKREGSSYEVICEAVAAAVAAI |
| | VKALKGCGTSEDEIAEIVARVISEVIRTLKESGSSYEVICECVARIVAEIVQALKES |
| | GTSEDEIAEIVARVISEVIRTLKESGSSYEVIKECVQRIVEEIVEALKRSGTSEDEI |
| | NEIVRRVKSEVERTLKESGSSGGSGSHHWGSGSHHHHHH |
| 177 | PHQFYVYQIDEHVAQLIEKFVRDISRREGTEVRFEKRDGQLEIEVKNLHEAQAIAIG |
| | IYIMILLLHQSGTSEDEIAEEIAKLIKGFIEHLKREGSSYEVICEAVAAAVAAIVKA |
| | LKGCGTSEDEIAEIVARVISEVIRTLKESGSSYEVICECVARIVAEIVQALKESGTS |
| | EDEIAEIVARVISEVIRTLKESGSSYEVIKECVQRIVEEIVEALKRSGTSEDEINEI |
| | VRRVKSEVERTLKESGSS |

name: 289B; alt. name: LHD289 B; type: single fusion monovalent

| | |
|---|---|
| 178 | GGSTVTFDITNISHEAIEIILYGVLGIAAMEGTEVTFHSERGQLQIEVKNLHEKQKR |
| | NIEKLIEAALRAQSPDPEDLKEAVRIAEELVRAHPGTPLAHAALQVILTAAEELAKL |
| | PDPEALKEAVKAAEKVVREQPGSNLAKKALEIILRAAEELAKLPDPEALKEAVKAAE |
| | KVVREQPGSELAKKALEIIERAAEELKKSPDPEAQKEAKKAEQKVREERPGSGGSGS |
| | HHWGSGSHHHHHH |
| 179 | TVTFDITNISHEAIEIILYGVLGIAAMEGTEVTFHSERGQLQIEVKNLHEKQKRNIE |
| | KLIEAALRAQSPDPEDLKEAVRIAEELVRAHPGTPLAHAALQVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |

name: 298B; alt. name: LHD298 B; type: single fusion monovalent

| | |
|---|---|
| 180 | GGSSHSFILGQASEEARQEIEEVVEAISRKLGTEVRFEKKDGTLHIEVKNLHDEYAQ |
| | LIADAILLIILAQESDDSEAKKVARLALEIVAQLPNTELAHEALKLAEEALKSTDSE |
| | ALKVVYLALRIVQQLPDTELAREALELAKEAVKSTDQEALKSVYEALQRVQDKPNTE |
| | EARESLERAKEDVKSTDGGSGSHHWGSGSHHHHHH |
| 181 | SHSFILGQASEEARQEIEEVVEAISRKLGTEVRFEKKDGTLHIEVKNLHDEYAQLIA |
| | DAILLIILAQESDDSEAKKVARLALEIVAQLPNTELAHEALKLAEEALKSTDSEALK |
| | VVYLALRIVQQLPDTELAREALELAKEAVKSTDQEALKSVYEALQRVQDKPNTEEAR |
| | ESLERAKEDVKSTD |

name: 317B; alt. name: LHD317 B; type: single fusion monovalent

| | |
|---|---|
| 182 | GGSDVEWRFTNVSEEEQEKLARFVLQVAQLAGTQVIFTTRPGELRIRVHNLDELLAL |
| | AIELYAQGLRLGDKHVQHLAKKAIEAILRGDRKLARFLLEAARAMSRATERPGSNLA |
| | KKALEEILRLAEELAKDPDPESLKAAVHCAEFVVRYQPGSNLAKKALEIILRAAEEL |
| | AKLPDPEALKEAVKAAEKVVREQPGSELAKKALEIIERAAEELKKSPDPEAQKEAKK |
| | AEQKVREERPGSGGSGSHHWGSGSHHHHHH |
| 183 | DVEWRFTNVSEEEQEKLARFVLQVAQLAGTQVIFTTRPGELRIRVHNLDELLALAIE |
| | LYAQGLRLGDKHVQHLAKKAIEAILRGDRKLARFLLEAARAMSRATERPGSNLAKKA |
| | LEEILRLAEELAKDPDPESLKAAVHCAEFVVRYQPGSNLAKKALEIILRAAEELAKL |
| | PDPEALKEAVKAAEKVVREQPGSELAKKALEIIERAAEELKKSPDPEAQKEAKKAEQ |
| | KVREERPGS |

name: 321B; alt. name: LHD321 B; type: single fusion monovalent

| | |
|---|---|
| 184 | GGSTVTFDITNIDDKSTKLIATAVIHIAGREGTTVHFQGHDGQLEIEVKNLHEKWKR |
| | LIEMLIEACRRAQDPDPESLKEAVRIAEELVRLHPGNMLAEAALKVILTAAEELAKL |
| | PDPEALKEAVKAAEKVVREQPGSNLAKKALEIILRAAEELAKLPDPEALKEAVKAAE |
| | KVVREQPGSELAKKALEIIERAAEELKKSPDPEAQKEAKKAEQKVREERPGSGGSGS |
| | HHWGSGSHHHHHH |
| 185 | TVTFDITNIDDKSTKLIATAVIHIAGREGTTVHFQGHDGQLEIEVKNLHEKWKRLIE |
| | MLIEACRRAQDPDPESLKEAVRIAEELVRLHPGNMLAEAALKVILTAAEELAKLPDP |
| | EALKEAVKAAEKVVREQPGSNLAKKALEIILRAAEELAKLPDPEALKEAVKAAEKVV |
| | REQPGSELAKKALEIIERAAEELKKSPDPEAQKEAKKAEQKVREERPGS |

name: TF3; alt. name: 274B 62 275A 10 101A; type: Connector
trivalent

| | |
|---|---|
| 186 | HHHHHHGSGSNTHFIVVHGTEEARQLAEEIVRLIAKALGTEVRFEKKDGLLSIEVKN |
| | LPEEAQRLIQKLLQLAVRIAAAAKSGDNDVLRKLAEDALRLAKEAEKLGDLGAAAVA |
| | ARLAVEAAKQAGDNDVLRKVAEQALRIAKEAEKQGNVEVAVKAARVAVEAAKQAGDN |
| | DVLRKVAEQALRIAKEALKQGNFRVAIEALKVAEEAAKQAGDQDVLKKVEELKLEIF |
| | AKAIEDLVRKMGVEMLVFKAGRAVIVVIRGLHPEQAKQLLRFVSQLAHDLGVTVTLT |
| | FHGDVVFILVLVGASEEEQKVMQLAIQLLARIIHEAKRRGVSEEALKAIAEFVAIVL |
| | EALKRAGILSEEALELATRLLKEVLENAQREGYDESEAIRAAAEALKRVAEAAKRAG |
| | ITSSEVLELAIRLIKEVVENAQREGYDISEAARAAAEAFKRVAEAAKRAGITSSEVL |
| | ELAIILIKLVVELAQRKGYDISEAARAAAELFKRLAEALKRAGKTSERALALLILLL |
| | AIEILVRDMGVTMETHPSGNEVKVVIKGLHIKQQRQLYRLVREAAKLLGVEVEIEVE |
| | GDTVTIVVRGGS |
| 187 | NTHFIVVHGTEEARQLAEEIVRLIAKALGTEVRFEKKDGLLSIEVKNLPEEAQRLIQ |
| | KLLQLAVRIAAAAKSGDNDVLRKLAEDALRLAKEAEKLGDLGAAAVAARLAVEAAKQ |
| | AGDNDVLRKVAEQALRIAKEAEKQGNVEVAVKAARVAVEAAKQAGDNDVLRKVAEQA |
| | LRIAKEALKQGNFRVAIEALKVAEEAAKQAGDQDVLKKVEELKLEIFAKAIEDLVRK |
| | MGVEMLVFKAGRAVIVVIRGLHPEQAKQLLRFVSQLAHDLGVTVTLTFHGDVVFILV |
| | LVGASEEEQKVMQLAIQLLARIIHEAKRRGVSEEALKAIAEFVAIVLEALKRAGILS |
| | EEALELATRLLKEVLENAQREGYDESEAIRAAAEALKRVAEAAKRAGITSSEVLELA |
| | IRLIKEVVENAQREGYDISEAARAAAEAFKRVAEAAKRAGITSSEVLELAIILIKLV |
| | VELAQRKGYDISEAARAAAELFKRLAEALKRAGKTSERALALLILLLAIEILVRDMG |
| | VTMETHPSGNEVKVVIKGLHIKQQRQLYRLVREAAKLLGVEVEIEVEGDTVTIVVRG |

name: TF10; alt. name: corrTF 274B d62 317A d52 101A; type:
Connector trivalent

| | |
|---|---|
| 188 | HHHHHHGSGSNTHFIVVHGTEEARQLAEEIVRLIAKALGTEVRFEKKDGLLSIEVKN |
| | LPEEAQRLIQKLLQLAVRIAAAAKSGDNDVLRKLAEDALRLAKEAEKLGDLGAAAVA |
| | ARLAVEAAKQAGDNDVLRKVAEQALRIAKEAEKQGNVEVAVKAARVAVEAAKQAGDN |
| | DVLRKVAEQALRIAKEAEKQGNVEVAIKALEVAAEAAAQAGDKDVLKKILEQLERLA |
| | ELAKKQGNKELAIKIFELFIKVIVALMGVRMLSHKGGNAVIVVIEGLHPSQAEQLLR |
| | LVHRIAKKAGVTVHLVFTGDIVVIMVVVGASEEEQELMHELVRLIAEALHEAKRLGA |
| | NEEFLEQLLKLLTLVVRAALRTGSDEARQALEELARIAKEALEEGNAELAKFAIRLL |
| | EWLARLYSGSDVASLAVKAIAKIAETALRNGNADTAKEAIQRLEDLARDYSGSDVAS |
| | LAVKAIAKIAETALRNGDADTAKEAIQRLEDLARDYSGSDVASLAVEAILRIALIAL |
| | ANGNEETAEEARKRLRELAEDYKGSEVAKLAESAERLIEILKIIAKTVRKMGVTMDV |
| | RPSGTEVEVVIKGLHIKQQRQLYRDVREAAKKLGVEVEIEVEGDTVTIVVRGGS |
| 189 | NTHFIVVHGTEEARQLAEEIVRLIAKALGTEVRFEKKDGLLSIEVKNLPEEAQRLIQ |
| | KLLQLAVRIAAAAKSGDNDVLRKLAEDALRLAKEAEKLGDLGAAAVAARLAVEAAKQ |
| | AGDNDVLRKVAEQALRIAKEAEKQGNVEVAVKAARVAVEAAKQAGDNDVLRKVAEQA |
| | LRIAKEAEKQGNVEVAIKALEVAAEAAAQAGDKDVLKKILEQLERLAELAKKQGNKE |
| | LAIKIFELFIKVIVALMGVRMLSHKGGNAVIVVIEGLHPSQAEQLLRLVHRIAKKAG |
| | VTVHLVFTGDIVVIMVVVGASEEEQELMHELVRLIAEALHEAKRLGANEEFLEQLLK |
| | LLTLVVRAALRTGSDEARQALEELARIAKEALEEGNAELAKFAIRLLEWLARLYSGS |
| | DVASLAVKAIAKIAETALRNGNADTAKEAIQRLEDLARDYSGSDVASLAVKAIAKIA |
| | ETALRNGDADTAKEAIQRLEDLARDYSGSDVASLAVEAILRIALIALANGNEETAEE |
| | ARKRLRELAEDYKGSEVAKLAESAERLIEILKIIAKTVRKMGVTMDVRPSGTEVEVV |
| | IKGLHIKQQRQLYRDVREAAKKLGVEVEIEVEGDTVTIVVRG |

name: DF275B0; alt. name: 275B 53 0 101A; type: Connector
bivalent

| | |
|---|---|
| 196 | MDVEWRYTNISEETQQKSAEFVLEIALRAGTGVTFTTRQGELQIQVHNLDELLAIAM |
| | LCYTLGLLLGDHRVQELAKRAVEAWERGDEERVKKLLIEALKRLVETAEEVVRERPG |
| | SNLAKLALEIILRAAEALARAEDPESLKEAVKAAEKVVREQPGSNLAKKALEIILRA |
| | AEELAKLPDPEALKEAVKAAEKVVREQPGSNLAKKALEIILRAAEELAKLPDPEALK |
| | EAVKAAEKVVREQPGSNLAKKALEIILRAAAALANLPDPESRKEADKAADKVRREQP |
| | GSELAVVAAIISAVARMGVKMELHPSGNEVKVVIKGLHIKQQRQLYRDVREAAKKAG |
| | VEVEIEVEGDTVTIVVRGGSGSGSSRGPYPYDVPDYA |
| 197 | DVEWRYTNISEETQQKSAEFVLEIALRAGTGVTFTTRQGELQIQVHNLDELLAIAML |
| | CYTLGLLLGDHRVQELAKRAVEAWERGDEERVKKLLIEALKRLVETAEEVVRERPGS |
| | NLAKLALEIILRAAEALARAEDPESLKEAVKAAEKVVREQPGSNLAKKALEIILRAA |
| | EELAKLPDPEALKEAVKAAEKVVREQPGSNLAKKALEIILRAAEELAKLPDPEALKE |
| | AVKAAEKVVREQPGSNLAKKALEIILRAAAALANLPDPESRKEADKAADKVRREQPG |
| | SELAVVAAIISAVARMGVKMELHPSGNEVKVVIKGLHIKQQRQLYRDVREAAKKAGV |
| | EVEIEVEGDTVTIVVRG |

name: C3-Hub; alt. name: C3 82; type: Cn

| | |
|---|---|
| 198 | CVEELLLLARAAHHSGTTVEEAYKLAKKLGISVKELLLLARAAHNSGTTVEEAYKLA |
| | LKLGISVEELLLLAKAAHYSGTTVEEAYKLALELGISVRELLLLAKAAHFAGRTVRE |
| | AYALCLALGALRLEDRARELIKEAEKKGDPEKLREALEALEEAVRLVEEAIKLRPDM |
| | DLAVEIAVRLARMLKRVAELLQELAKKTGDPELLKLALRALEVAVRAVELAIKSNPD |
| | NDEAVETAVRLARELAKVAEELIERAKKTGDKELLKLAKRALEVAMRAVSLALKSNP |
| | DNEEARRVAAELVLLVIRAAVIEMGVTMEEHRSGNRVKVVIKGLHESQQEQLLEDVL |
| | RAAEIAGVRVRIRFKGDTVTIVVEGSGSGSHHHHHH |
| 199 | CVEELLLLARAAHHSGTTVEEAYKLAKKLGISVKELLLLARAAHNSGTTVEEAYKLA |
| | LKLGISVEELLLLAKAAHYSGTTVEEAYKLALELGISVRELLLLAKAAHFAGRTVRE |
| | AYALCLALGALRLEDRARELIKEAEKKGDPEKLREALEALEEAVRLVEEAIKLRPDM |
| | DLAVEIAVRLARMLKRVAELLQELAKKTGDPELLKLALRALEVAVRAVELAIKSNPD |
| | NDEAVETAVRLARELAKVAEELIERAKKTGDKELLKLAKRALEVAMRAVSLALKSNP |
| | DNEEARRVAAELVLLVIRAAVIEMGVTMEEHRSGNRVKVVIKGLHESQQEQLLEDVL |
| | RAAEIAGVRVRIRFKGDTVTIVVEG |

In another aspect the disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), non-naturally occurring polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the disclosure provides heterodimers, comprising two polypeptides or fusion proteins according to any embodiment herein, wherein the two polypeptides are capable of self-assembly to form a heterodimer. As described in the examples that follow, the polypeptides cab form beta sheet mediated heterodimers, which enable the generation of a wide variety of structurally well-defined asymmetric assemblies. Crystal structures of the heterodimers are very close to the design models, and unlike previously designed orthogonal heterodimer sets, the subunits are stable, folded and monomeric in isolation and rapidly assemble upon mixing. Rigid fusion of individual heterodimer halves to repeat proteins yields central assembly hubs that can bind two or three different proteins across different interfaces. We use these connectors to assemble linearly arranged hetero-oligomers with up to 6 unique components, branched hetero-oligomers, closed C4-symmetric two-component rings, and hetero-oligomers assembled on a cyclic homo-oligomeric central hub, and demonstrate such complexes can readily reconfigure through subunit exchange. Thus, the heterodimers can be used, for example, to generate asymmetric reconfigurable protein systems. Such systems may, for example, include fusion to target proteins of interest to co-localize and position multiple copies of the same target fusion for any suitable purpose such as to target multiple copies of therapeutic proteins of interest for therapeutic treatment.

In one embodiment, the two polypeptides or fusion proteins are a Chain A and Chain B pair as listed in any of Tables 1-3.

In various embodiments, by way of example, the Chain A and Chain B pair may comprise an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of selected from the following pairs (Chain A listed first; Chain B listed second), not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity:
(a) one of SEQ ID NOS:1-5 and SEQ ID NO: 6;
(b) SEQ ID NO:7 and SEQ ID NO:8;
(c) SEQ ID NO:9 and SEQ ID NO: 10;
(d) SEQ ID NO:11 and SEQ ID NO: 12;
(e) SEQ ID NO:13 and SEQ ID NO: 14;
(f) SEQ ID NO:15 and SEQ ID NO: 16;
(g) SEQ ID NO:17 and SEQ ID NO: 18;
(h) SEQ ID NO:19 and SEQ ID NO:20;
(i) SEQ ID NO:21 and SEQ ID NO:22;
(j) SEQ ID NO:23 and SEQ ID NO:24;
(k) SEQ ID NO:25 and SEQ ID NO:26; and
(l) SEQ ID NO:27 and SEQ ID NO:28.

In other embodiments, by way of example, the Chain A and Chain B pair may comprise the amino acid sequence selected from the following pairs (Chain A listed first; Chain B listed second):
(a) one of SEQ ID NOS:29-32 and SEQ ID NO:33;
(b) SEQ ID NO:190 and SEQ ID NO:191;
(c) SEQ ID NO:35 and SEQ ID NO:36;
(d) SEQ ID NO:37 and SEQ ID NO:38;
(e) SEQ ID NO:39 and SEQ ID NO:40;
(f) SEQ ID NO:41 and SEQ ID NO:42;
(g) SEQ ID NO:43 and SEQ ID NO:44;
(h) SEQ ID NO:46 and SEQ ID NO:47;
(i) SEQ ID NO:48 and SEQ ID NO:49;
(j) SEQ ID NO:50 and SEQ ID NO:51;
(k) SEQ ID NO:52 and SEQ ID NO:53;
(l) SEQ ID NO:54 and SEQ ID NO: 55;
(m) one of SEQ ID NO:56-59 and SEQ ID NO:60;
(n) SEQ ID NO:61 and SEQ ID NO: 191;
(o) SEQ ID NO:62 and SEQ ID NO: 63;
(p) SEQ ID NO:64 and SEQ ID NO:65;
(q) SEQ ID NO:66 and SEQ ID NO:67;
(r) SEQ ID NO: 68 and SEQ ID NO:69;
(s) SEQ ID NO:70 and SEQ ID NO: 71;
(t) SEQ ID NO:72 and SEQ ID NO:73;
(u) SEQ ID NO:74 and SEQ ID NO:75; and
(v) SEQ ID NO:76 and SEQ ID NO:77.

As described in the examples that follow, the inventors have provided numerous examples of such heterodimers.

In another embodiment, the disclosure provides asymmetric hetero-oligomeric assemblies comprising a plurality (2, 3, 4, 5, 6, 7, 8, 9, 10, or more) of the heterodimers of the disclosure. As shown in the examples, the inventors have provided numerous exemplary such assemblies, including linearly arranged hetero-oligomers with up to 6 unique components, branched hetero-oligomers, closed C4-symmetric two-component rings, and hetero-oligomers assembled on a cyclic homo-oligomeric central hub, and demonstrate such complexes can readily reconfigure through subunit exchange. Exemplary embodiments are as detailed in Tables 6 and 7. In some embodiments, linear heterotrimers comprise a central component that is a repeat protein fused to LHD monomers at both termini (bivalent connector); Outer component 1 binds to the LHD monomer at the N-terminus of the central component, outer component 2 binds to the LHD monomer at the C-terminus of the central component. Names of the components refer to proteins refer to the components described above in Table 5. By way of non-limiting example, the first row in Table 5 lists the trimeric assembly 274A53-DFB0-101B62. This trimeric assembly comprises 274A53 (SEQ ID NO:162 or 163)-DFB0 (SEQ ID NO:100 or 100)-101B62 (SEQ ID NO:136 or 137). Those of skill in the art can readily determine the sequences of components of the other assemblies in Table 6, each of which is detailed in the examples that follow.

TABLE 6
Exemplary assemblies

| | Outer | | Outer | |
|---|---|---|---|---|
| Trimers | Comp. 1 | Ctr. Comp. | Comp. 2 | Comment |
| 274A53-DFB0-101B62 | 274A53 | DFB0 | 101B62 | |
| 275B_DF275A-1_206B62-2 | 275B | DF275A-1 | 206B62-2 | |
| 274A53-DF206-206A54 | 274A53 | DF206 | 206A54 | |
| 274A53-DF202-202B57 | 274A53 | DF202 | 202B57 | |
| 274B53_DFA-1_101B62 | 274B53 | DFA-1 | 101B62 | |
| 274A53_DFB-1_101B62 | 274A53 | DFB-1 | 101B62 | |
| 284A82-DF284-101A53 | 284A82 | DF284 | 101A53 | |
| 274B-DFA0-101B | 274B | DFA0-GFP | 101B | |
| 274B-DFA0-101B4 | 274B | DFA0-GFP | 101B4 | |
| 274B53-DFA0-GFP-101B4 | 274B53 | DFA0-GFP | 101B4 | |
| 284A82_DF284_DFA-GFP_{—} | 284A82 | DF284 | DFA-GFP | control |
| DF206_DF275A_275B | | | | ABC |
| 284A82_DF284_DFA-GFP_{—} | DF284 | DFA-GFP | DF206 | control |
| DF206_DF275A_275B | | | | BCD |
| 284A82_DF284_DFA-GFP_{—} | DFA-GFP | DF206 | DF275A-1 | control |
| DF206_DF275A_275B | | | | CDE |
| 101B4_DFA-GFP_DF206_{—} | 101B4 | DFA-GFP | DF206 | control |
| DF275A-1_275B | | | | ABC |
| | | | | pentamer |
| 101B4_DFA-GFP_DF206_{—} | DF206 | DF275A-1 | 275B | control |
| DF275A-1_275B | | | | CDE |
| | | | | pentamer |
| 101B4-DFA0-DF202-202B57 | 101B4 | DFA0 | DF202 | control |
| | | | | ABC |
| | | | | tetramer |
| 101B4-DFA0-DF202-202B57 | DFA0 | DF202 | 202B57 | control |
| | | | | BCD |
| | | | | tetramer |
| 101B82-DFA0-DF202-202B57 | 101B82 | DFA0 | DF202 | control |
| | | | | ABC |
| | | | | tetramer |
| 101B82-DFA0-DF202-202B57 | DFA0 | DF202 | 202B57 | control |
| | | | | BCD |
| | | | | tetramer |
| 274A53_DFx_317B | 274A53 | DFx | 317B | |
| Linear heterooligomeric | | | | |
| assemblies with more than | | | | |
| three components can be | | | | |
| generated by using more | | | | |
| than one bivalent connector: | | | | |
| tetramers | | | | |
| 101B4-DFA-DF202-202B57 | | | | |
| 101B82-DFA-DF202-202B57 | | | | |
| 101B62-DFA-DFB-101B62 | | | | |
| 101B62-DFA-1-DFB-101B62 | | | | |
| 101B62-DFA-DFB-1-101B62 | | | | |
| 101B62-DFA-1-DFB-1-101B62 | | | | |
| 101B4-DFA-DF206-DF275A-1 | | | | control |
| | | | | pentamer |
| DFA-DF206-DF275A-1-275B | | | | control |
| | | | | pentamer |
| 284A82-DF284B-DFA-DF206 | | | | control |
| | | | | hexamer |
| DFA-DF206-DF275A-1-275B | | | | control |
| | | | | hexamer |
| DF284B-DFA-DF206-DF275A-1 | | | | control |
| | | | | hexamer |
| pentamers | | | | |
| 101B4-DFA-DF206-DF275A- | | | | |
| 1-275B | | | | |
| 101B62-DFA-DF206-DF275A- | | | | |
| 1-275B | | | | |
| 284A82-DF284B-DFA-DF206- | | | | control |
| DF275A-1 | | | | hexamer |
| DF284B-DFA-DF206-DF275A- | | | | control |
| 1-275B | | | | hexamer |
| hexamers | | | | |
| 284A82-DF284B-DFA-DF206- | | | | |
| DF275A-1-275B | | | | |

As will be understood by those of skill in the art, many such complexes can be generated. In various non-limiting embodiments, such complexes may include those described in Table 7, which lists potential linear oligomers that could be assembled from the experimentally verified components listed in Table 5. The assemblies in Table 7 are grouped by connectivity, meaning that for each line of the table any component 1 can be combined with any component 2, any component 3, etc.

TABLE 7

| | | list of exemplary components that can be used at each position | | | | | |
|---|---|---|---|---|---|---|---|
| | | component | component | component | component | component | component |
| | type | 1 | 2 | 3 | 4 | 5 | 6 |
| trimers | | | | | | | |
| DFA | A | 274B, 274B53, | DFA0, | 101B, 101B4, | na | na | na |
| | B | 274B62, | DFA-1 | 101B8, | | | |
| | C | 274B82, DF202, | | 101B14, | | | |
| | | DF206 , DFx | | 101B62, | | | |
| | | | | 101B82, DF284 | | | |
| DFB | A | 274A, 274A53, | DFB0, | 101B, 101B4, 101B8, 101B14, | | | |
| | B | 274A64, 274A76 | DFB-1 | 101B62, 101B82, DF284 | | | |
| | C | | | | | | |
| DF202 | A | 274A, 274A53, | DF202 | 202B, 202B57, | | | |
| | B | 274A64, | | 202B64 | | | |
| | C | 274A76, DFA0, | | | | | |
| | | DFA-1 | | | | | |
| DF206 | A | 274A, 274A53, | DF206 | 206A, 206A54, | | | |
| | B | 274A64, | | DF275A-1 | | | |
| | C | 274A76, DFA0, | | | | | |
| | | DFA-1 | | | | | |
| DFx | A | 274A, 274A53, | DFx | 317B | | | |
| | B | 274A64, | | | | | |
| | C | 274A76, DFA0, | | | | | |
| | | DFA-1 | | | | | |
| DF275A-1 | A | 275B | DF275A-1 | 206B, 206B62-1, 206B62-2, | | | |
| | B | | | DF206 | | | |
| | C | | | | | | |
| DF284B | A | 284A, 284A82 | DF284B | 101A, 101A10, 101A21, 101A52, | | | |
| | B | | | 101A53, DFA0, DFA-1, DEB0, | | | |
| | C | | | DFB-1, DF321 | | | |
| DF321 | A | 321A | DF321 | 101B, 101B4, 101B8, 101B14, | | | |
| | B | | | 101B62, 101B82, DF284 | | | |
| | C | | | | | | |
| DF0 | A | 29B, 29B53 | DF0 | 101B, 101B4, 101B8, 101B14, | | | |
| | B | | | 101B62, 101B82, DF284 | | | |
| | C | | | | | | |
| RingA | A | 29A, 29A53 | RingA | 101B, 101B4, 101B8, 101B14, | | | |
| | B | | | 101B62, 101B82, DF284 | | | |
| | C | | | | | | |
| RingB | A | 29B, 29B53 | RingB | 101A, 101A10, 101A21, | | | |
| | B | | | 101A52, | | | |
| | C | | | 101A53, DF321 | | | |
| tetramers | | | | | | | |
| 101B-DFA- | A | 101B, 101B4, | DFA0, | DFB0, DFB-1 | 101B, 101B4, | | |
| DFB-101B | B | 101B8, 101B14, | DFA-1 | | 101B8, 101B14, | | |
| | C | 101B62, | | | 101B62, 101B82, | | |
| | A | 101B82, DF284B | | | DF284B | | |
| 101B-DFA- | A | 101B, 101B4, | DFA0, | DF202 | 202B, | | |
| DF202- | B | 101B8, 101B14, | DFA-1 | | 202B57, | | |
| 202B | C | 101B62, | | | 202B64 | | |
| | D | 101B82, DF284B | | | | | |
| 101B-DFA- | A | 101B, 101B4, | DFA0, | DF206 | 206A, | | |
| DF206- | B | 101B8, 101B14, | DFA-1 | | 206A54, | | |
| 206A | C | 101B62, | | | DF275A-1 | | |
| | D | 101B82, DF284B | | | | | |
| 274A- | A | 274A, 274A53, | DF206 | DF275A-1 | 275B | | |
| DF206- | B | 274A64, | | | | | |
| DF275A-1- | C | 274A76, DFA0, | | | | | |
| 275B | D | DFA-1 | | | | | |
| 284A- | A | 284A, 284A82 | DF284B | DEA0, DFA-1 | 274B, 274B53, | | |
| DF284B- | B | | | | 274B62, | | |
| DFA-274B | C | | | | 274B82, DF202, | | |
| | D | | | | DF206, DFx | | |
| 284A- | A | 284A, 284A82 | DF284B | DFB0, DFB-1 | 274A, | | |
| DF284B- | B | | | | 274A53, | | |
| DFB-274A | C | | | | 274A64, | | |
| | D | | | | 274A76 | | |
| 284A- | A | 284A, 284A82 | DF284B | DF321 | 321A | | |
| DF284B- | B | | | | | | |
| DF321- | C | | | | | | |
| 321A | D | | | | | | |
| 284A- | A | 284A, 284A82 | DF284B | DF0 | 29B, | | |
| DF284B- | B | | | | 29B53 | | |
| DF0-29B | C | | | | | | |
| | D | | | | | | |
| 284A- | A | 284A, 284A82 | DF284B | RingA | 29A, | | |
| DF284B- | B | | | | 29A53 | | |
| ringA-29A | C | | | | | | |
| | D | | | | | | |
| 321A- | A | 321A | DF321 | RingB | 29B, | | |
| DF321- | B | | | | 29B53 | | |
| ringB-29B | C | | | | | | |
| | D | | | | | | |
| 317B-DFx- | A | 317B | DFx | DFA0, DFA-1 | 101B, 101B4, | | |
| DFA-101B | B | | | | 101B8, 101B14, | | |
| | C | | | | 101B62, 101B82, | | |
| | D | | | | DF284B | | |
| pentamers | | | | | | | |
| 101B-DFA- | A | 101B, 101B4, | DFA, | DF206 | DF275A-1 | 275B | |
| DF206- | B | 101B8, 101B14, | DFA-1 | | | | |
| DF275A-1- | C | 101B62, | | | | | |
| 275B | D | 101B82, DF284B | | | | | |
| | E | | | | | | |
| 284A- | A | 284A, 284A82 | DF284B | DFA, DFA-1 | DF206 | 206A, | |
| DF284B- | B | | | | | 206A54, | |
| DFA- | C | | | | | DF275A-1 | |
| DF206- | D | | | | | | |
| 206A | E | | | | | | |
| 317B-DFx- | A | 317B | DFx | DEA0, DFA-1 | DF284B | 284A, | |
| DFA- | B | | | | | 284A82 | |
| DF284B- | C | | | | | | |
| 284A | D | | | | | | |
| | E | | | | | | |
| hexamers | | | | | | | |
| 284A- | A | 284A, 284A82 | DF284B | DFA, DFA-1 | DF206 | DF275A-1 | 275B |
| DF284B- | B | | | | | | |
| DFA- | C | | | | | | |
| DF206- | D | | | | | | |
| DF275A-1- | E | | | | | | |
| 275B | F | | | | | | |

Thus, in another embodiment, the disclosure provides assemblies comprising components as provided in individual rows of Table 6 or 7, wherein each component comprises an amino acid sequence at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 90-189 and 196-199, or SEQ ID NOS: 90-189, not including any functional domains added (whether N-terminal, C-terminal, or internal other than within the interface region), and wherein the 1, 2, 3, 4, or 5 N-terminal and/or C-terminal amino acid residues, as well as any N-terminal methionine residue, may be present or absent when considering the percent identity.

In another embodiment, the disclosure provides methods for making a heterodimer, comprising mixing two or more of the polypeptides or fusion proteins of any embodiment, resulting in self-assembly of the heterodimer. as described in detail in the examples that follow.

The disclosure also provides methods for designing heterodimers and heterodimer-forming polypeptides, comprising any steps or combination of steps as detailed in the examples that follow.

In another aspect, the present disclosure provides pharmaceutical compositions, comprising one or more polypeptides, fusion proteins, heterodimers, compositions, nucleic acids, expression vectors, and/or host cells of the disclosure and a pharmaceutically acceptable carrier. The pharmaceutical compositions of the disclosure can be used, for example, when using the components to target therapeutic proteins of interest for therapeutic treatment. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

### EXAMPLES

Asymmetric multi-protein complexes that undergo subunit exchange play central roles in biology, but present a challenge for protein design. The individual components must contain interfaces enabling reversible addition to and dissociation from the complex, but be stable and well behaved in isolation. Here we employ a set of implicit negative design principles to generate beta sheet mediated heterodimers that enable the generation of a wide variety of structurally well-defined asymmetric assemblies. Crystal structures of the heterodimers are very close to the design models, and unlike previously designed orthogonal heterodimer sets, the subunits are stable, folded and monomeric in isolation and rapidly assemble upon mixing. Rigid fusion of individual heterodimer halves to repeat proteins yields central assembly hubs that can bind two or three different proteins across different interfaces. We use these connectors to assemble linearly arranged hetero-oligomers with up to 6 unique components, branched hetero-oligomers, closed C4-symmetric two-component rings, and hetero-oligomers assembled on a cyclic homo-oligomeric central hub, and demonstrate such complexes can readily reconfigure through subunit exchange. Our approach provides a general route to designing asymmetric reconfigurable protein systems.

The design of reconfigurable asymmetric assemblies is a more difficult challenge, as there is no symmetry “bonus” favoring the target structure (as is attained for example in the closing of an icosahedral cage), and because the individual subunits must be stable and soluble proteins in isolation in order to reversibly associate or dissociate. Reconfigurable asymmetric protein assemblies could in principle be constructed using a modular set of protein-protein interaction pairs (heterodimers), provided first, that the interaction pairs are specific, second, that individual components are stable both in isolation and in complex so they can be added and removed, and third, that they can be rigidly fused to other components without changing the dimerization properties. Rigid fusion, as opposed to fusion by flexible linkers, is important to program the assembly of structurally well-defined complexes, as most higher order natural protein complexes have, despite their reconfigurability, distinct overall shapes that are critical for their function.

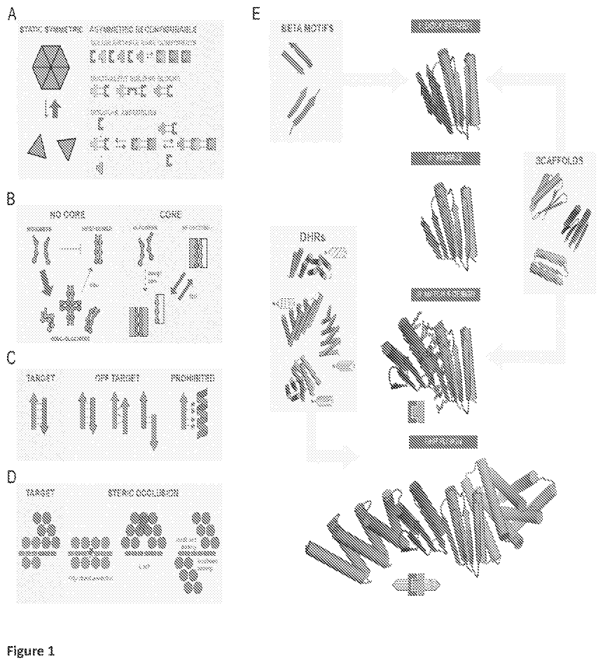

We set out to design sets of interacting protein pairs with properties required for subsequent programming of reconfigurable protein assemblies (FIG. 1A). The first challenge to overcome is the systematic design of proteins with interaction surfaces that drive association with cognate partners, but not self-association. This is not straightforward, as hydrophobic interactions provide a driving force for protein assembly, but these same hydrophobic residues can then mediate undesired self-self interactions.

We sought to use implicit negative design by introducing three properties that collectively make self-associated states unlikely to have low free energy: First, we aimed for well-folded individual protomers stabilized by substantial hydrophobic cores; this property limits the formation of slowly-exchanging homo-oligomers (FIG. 1B). Second, we constructed interfaces in which each protomer has a mixed alpha-beta topology and contributes one exposed beta strand to the interface, giving rise to a continuous beta sheet across the heterodimer interface (FIG. 1C). The exposed polar backbone atoms of this “edge strand” limit undesired self-association to arrangements that pair the beta edge strands; most other homomeric arrangements result in energetically unfavorable burial of the polar backbone atoms on the beta edge strand and hence are unlikely to form (FIG. 1C). Third, we incorporated structural elements likely to clash in undesired homomeric states (steric occlusion). The restrictions in possible undesired states resulting from strategies 1 and 2 make it possible to explicitly model the limited number of homo-oligomeric states, and hence to explicitly design in additional elements likely to sterically occlude such states (FIG. 1D).

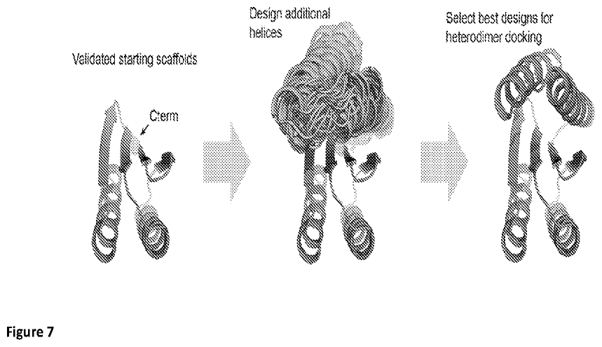

To implement these properties in actual proteins, we chose to start with a set of mixed alpha/beta scaffolds. The selected designs contain sizable hydrophobic cores, exposed edge strands required for beta sheet extension and one terminal helix as needed for rigid helical fusion (FIG. 1E). Using blueprint-based backbone building, we designed additional helices at the other terminus for a subset of the scaffolds to enable rigid fusion at both the N and C termini (FIG. 7). Heterodimers with beta sheets extending across the interface were generated by superimposing one of the two strands from each of a series of paired beta strand templates on an edge beta strand of each scaffold (FIG. 1E, top), and then optimizing the rigid body orientation and the internal geometry of the partner beta strand to maximize hydrogen bonding interactions across the interface (FIG. 1E, second row). This generates a series of disembodied beta strands forming an extended beta sheet for each scaffold; for each of these, an edge beta strand from a second scaffold was superimposed on the disembodied beta strand to form an extended sheet-on-sheet interface (FIG. 1E, third row). The interface sidechain-sidechain interactions in the resulting protein-protein docks were optimized using Rosetta™ combinatorial sequence design. To limit excessive hydrophobic interactions, we either generated explicit hydrogen bond networks across the heterodimer interface, or used compositional constraints to encourage the use of polar residues while penalizing buried unsatisfied polar groups. This resulted in interfaces that, outside of the polar hydrogen bonding of the beta strands, contained both hydrophobic interactions and polar networks. To further disfavor unwanted homodimeric interactions (FIG. 1D, right panel), and to facilitate incorporation of the heterodimeric building blocks into higher order assemblies, we rigidly fused designed helical repeat proteins (DHRs) to terminal helices. Designed heterodimers were selected for experimental characterization based on binding energy, the number of buried unsatisfied polar groups, buried surface area and shape complementarity (see methods).

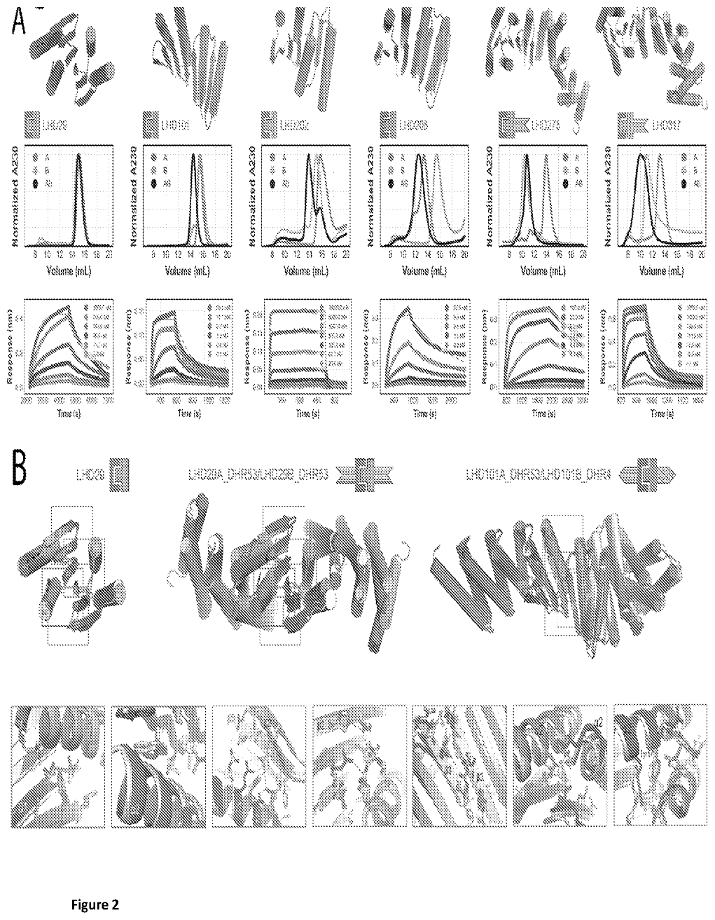

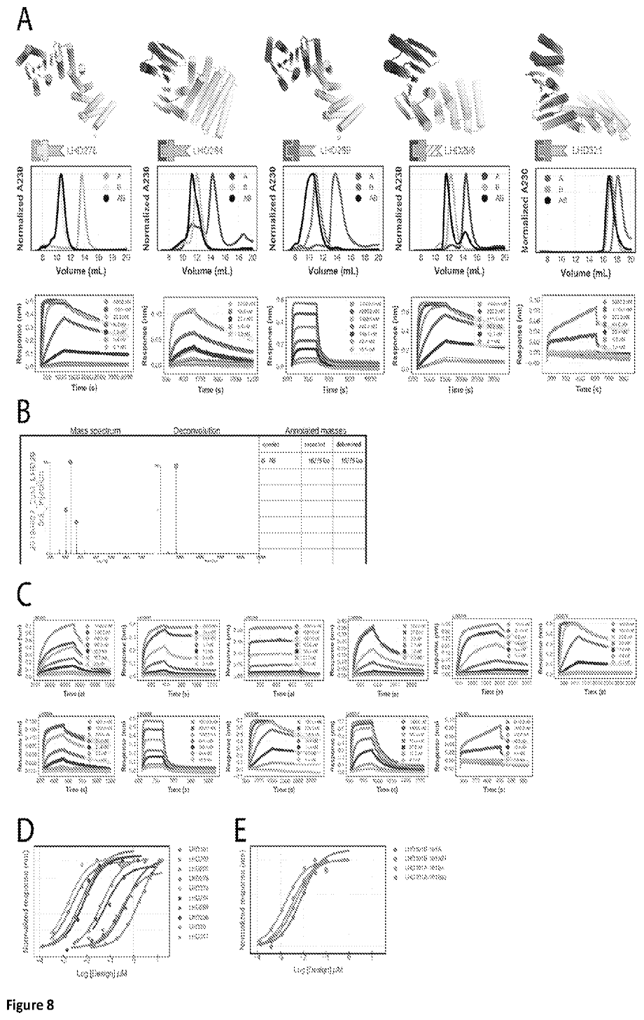

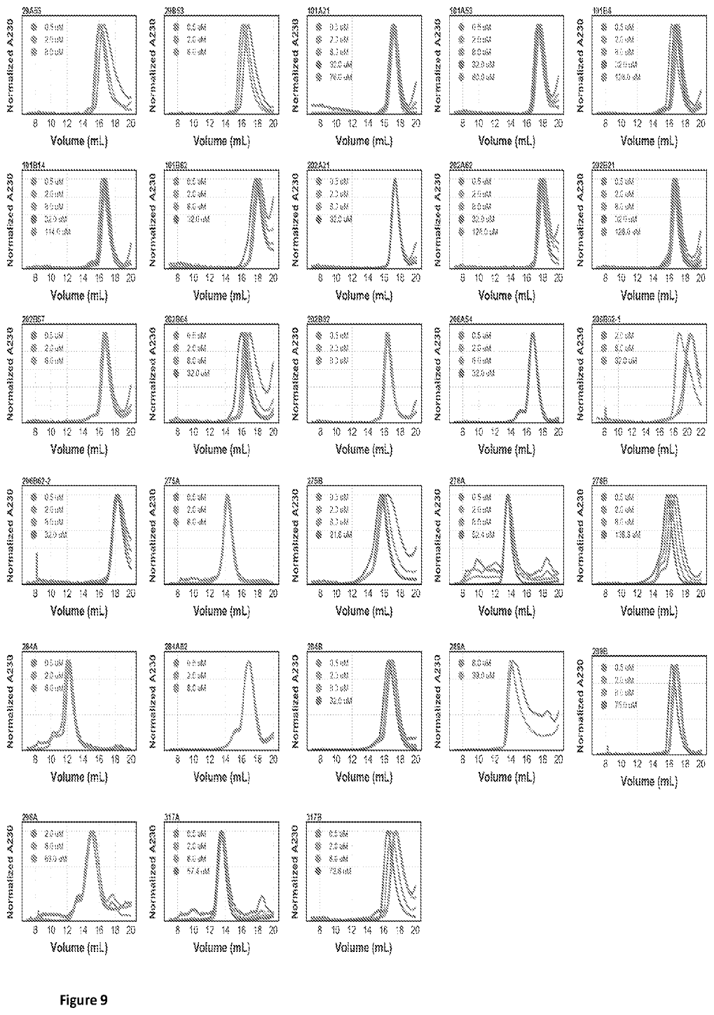

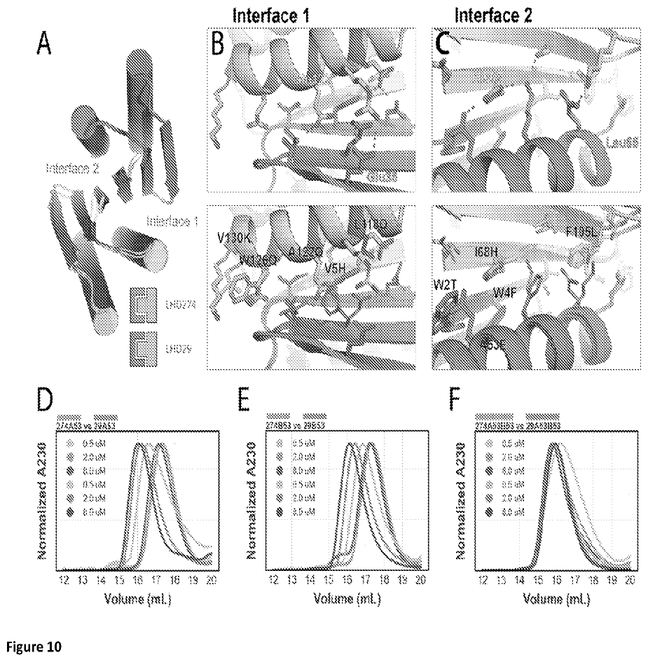

We co-expressed the selected heterodimers in K coil using a bicistronic expression system encoding one of the two protomers with a C-terminal polyhistidine tag and the other either untagged or GFP-tagged at the N-terminus. Complex formation was initially assessed using nickel affinity chromatography; designs for which both protomers were present in SDS-PAGE after nickel pulldown were subsequently subjected to size exclusion chromatography (SEC) and liquid chromatography-mass spectrometry (LC/MS). Of the 238 tested designs, 71 passed the bicistronic screen and were selected for individual expression of protomers. Of these, 32 formed heterodimers from individually purified monomers as confirmed by SEC, native MS, or both (FIG. 2A, FIG. 8). In SEC titration experiments, some protomers were monomeric at all injection concentrations, while others self-associated at higher concentrations (FIG. 9). Both LHD101 protomers and their fusions were monomeric even at injection concentrations above 100 μM (FIG. 9). LHD275A, LHD278A, LHD317A, and a redesigned version of LHD29 with a more polar interface (LHD274) were also predominantly monomeric (FIG. 9; FIG. 10). Designs for which isolated protomers were poorly expressed, polydisperse in SEC or did not yield stable, soluble and functional rigid DHR fusions were discarded together with designs that were very similar to other designs, but otherwise behaved well. After this stringent selection, we were left with a set of 11 heterodimers spanning three main structural classes (FIG. 2A, FIG. 8A). In class one, the central extended beta sheet is buttressed on opposite sides by helices that contribute additional interface interactions (LHDs 29 and 202 in FIG. 2A), in class two the helices that provide additional interactions are on the same side of the extended central sheet (LHDs 101 and 206 in FIG. 2A), and in the third class, both sides of the central beta sheet extension are flanked by helices (LHDs 275 and 317 in FIG. 2A).

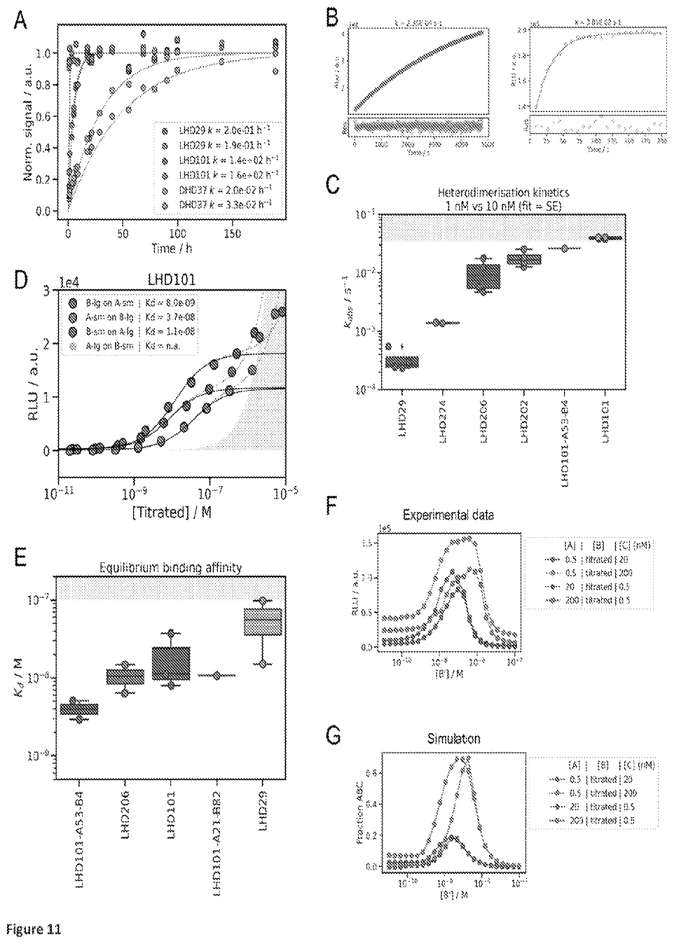

We monitored the kinetics of heterodimer formation and dissociation through biolayer interferometry (BLI) (FIG. 2A, FIG. 8A,C and table 8) by immobilizing individual biotinylated protomers onto streptavidin coated sensors and adding the designed binding partner. Unlike previously designed heterodimers, binding reactions equilibrated rapidly. Differences in off rates indicate that the heterodimers span a range of affinities (FIG. 8D and table 8). Association rates were quite fast and ranged from 10⁶M⁻¹ s⁻¹ for the fastest heterodimer to 10²M⁻¹ s⁻¹ for the slowest heterodimer LHD29; even LHD29 equilibrated an order of magnitude faster than the fastest associating designed helical hairpin heterodimer (FIG. 2A, FIG. 11A, Table 9). For LHD101 and LHD206 we confirmed BLI measurements in a split luciferase-based binding assay performed in E. coli lysates. The Kd's agreed well with those from BLI, showing that heterodimer association is not affected by high concentrations of non-cognate proteins (FIG. 11D,E and Table 10).

TABLE 8
Fitted values biolayer interferometry binding assays

| | Steady state fits | | Kinetic fits | | | | |
|---|---|---|---|---|---|---|---|
| Design | K_{D} (nM) | R-sqr | K_{D} (nM) | kₒₙ (M⁻¹ s⁻¹) | k_{off} (s⁻¹) | chi-sqr | R-sqr |
| LHD29¹ | 310 ± 120 | 0.91 | 985 ± 6.0 | 6.9 · 10² ± 4 | 6.8 · 10⁻⁴ ± | 6.7 | 0.98 |
| | | | | | 1.1 · 10⁻⁶ | | |
| LHD101 | 9.5 ± 0.76 | 0.99 | 1.9 ± 0.04 | 2.2 · 10⁶ ± | 4.3 . 10⁻³ ± | 0.21 | 0.97 |
| | | | | 4.0 · 10⁴ | 2.1 · 10⁻⁵ | | |
| LHD202 | 2400 ± 170 | 0.99 | 4800 ± 250 | 6.0 · 10⁴ ± | 2.9 · 10⁻¹ ± | 0.03 | 0.99 |
| | | | | 3.0 · 10³ | 0.05 | | |
| LHD206 | 8.4 ± 1.6 | 0.97 | 2.8 ± 0.02 | 2.7 · 10⁵ ± | 7.5 · 10⁻⁴ ± | 0.8 | 0.99 |
| | | | | 1.9 · 10³ | 2.2 · 10⁻⁶ | | |
| LHD274 | nd | nd | nd | nd | nd | nd | nd |
| LHD275 | 4.5 ± 0.22 | 0.99 | 2.9 ± 0.01 | 1.4 · 10⁵ ± | 4.1 · 10⁻⁴ ± | 0.76 | 0.99 |
| | | | | 4.6 · 10² | 1.1 · 10⁻⁶ | | |
| LHD278 | 3.4 ± 0.69 | 0.98 | 0.8 ± 0.003 | 2.9 · 10⁵ ± | 2.2 · 10⁻⁴ ± | 2.8 | 0.99 |
| | | | | 1 · 10³ | 3.6 · 10⁻⁷ | | |
| LHD284 | 97 ± 13 | 0.99 | 8.9 ± 0.13 | 1.3 · 10⁵ ± | 1.2 · 10⁻³ ± | 0.06 | 0.99 |
| | | | | 1.7 · 10³ | 6.7 · 10⁻⁶ | | |
| LHD289 | 610 ± 120 | 0.97 | 1080 ± 39 | 5.3 · 10⁴ ± | 5.7 · 10⁻² ± | 0.99 | 0.99 |
| | | | | 1.9 · 10³ | 5.8 · 10⁻⁴ | | |
| LHD298 | 16 ± 3 | 0.97 | 3.5 ± 0.01 | 6.4 · 10⁴ ± | 2.2 · 10⁻⁴ ± | 6.4 | 0.99 |
| | | | | 1.0 · 10² | 5.9 · 10⁻⁷ | | |
| LHD317 | 56 ± 2.3 | 0.99 | 34.7 ± 0.05 | 1.5 · 10⁵ ± | 5.1 · 10⁻³ ± | 4.7 | 0.99 |
| | | | | 2.1 · 10³ | 1.6 · 10⁻⁵ | | |
| LHD321 | nd | nd | nd | nd | nd | nd | nd |

¹Homodimerization of both LHD29 protomers under BLI conditions make Kd determination unreliable. Kd from split luciferase assay (FIG. 11) is more reliable as the experiment was performed under dilute conditions where homodimerization is minimized.
nd: not determined

TABLE 9
Fitted rate constants for heterodimerization reactions performed
at 1 nM vs. 10 nM in lysate. Errors indicate standard deviations.

| Design | k_{obs} (s⁻¹) |
|---|---|
| DHD37*^{, 1} | 7 ± 3 · 10⁻⁶ |
| LHD29 | 3 ± 1 · 10⁻⁴ |
| LHD29* | 5.5 ± 2 · 10⁻⁵ |
| LHD274 | 1.40 ± 0.01 · 10⁻³ |
| LHD206 | 1.0 ± 0.5 · 10⁻² |
| LHD202 | 1.8 ± 0.5 · 10⁻² |
| LHD101-A53-B4 | 2.6 · 10⁻² |
| LHD101 | 4.0 ± 0.1 · 10⁻² |
| LHD101* | 4.2 ± 0.4 · 10⁻² |

¹(Chen et al. 2019).
*Experiments performed with purified proteins, and reactions monitored by taking manual time-points as described in Materials and Methods and Supplementary Materials and Methods.

TABLE 10
Fitted equilibrium dissociation constants for binding curves
collected in lysate. Errors indicate standard deviations.

| Design | K_{d} (M) |
|---|---|
| LHD101 | 2 ± 1 · 10⁻⁸ |
| LHD206 | 1.1 ± 0.4 · 10⁻⁸ |
| LHD101-A21-B82 | 1.1 · 10⁻⁸ |
| LHD29 | 6 ± 4 · 10⁻⁸ |
| LHD101-A53-B4 | 4 ± 1 · 10⁻⁹ |

We determined the crystal structures of two class one designs, LHD29 (2.2 Å) and LHD29A53/B53 (2.6 Å) in which both protomers are fused to DHR53 (FIG. 2B and table 10). In the central extended beta sheet, the LHD29 design closely matches the crystal structure (FIG. 2B, red and green box). Aside from backbone beta sheet hydrogens bonds, this part of the interface is supported by primarily hydrophobic packing interactions between the side chains of each interface beta edge strand. The two flanking helices on opposite sides of the central beta sheet (FIG. 2B blue and orange box) contribute predominantly polar contacts to the interface, and are also very similar in the crystal structure and design model. Apart from crystal contact induced subtle backbone rearrangements in strand 2 of LHD29B, that promote the formation of a polar interaction network (FIG. 2B blue box), most interface sidechain-sidechain interactions agree well with the design model. Similar to the unfused LHD29, the interface of LHD29A53/B53 closely resembles the designed model; at the fusion junction and repeat protein regions, deviations are slightly larger.

TABLE 11
Crystallographic data collection and refinement.

| | LHD29 | LHD29A53/B53 | LHD101A53/B4 |
|---|---|---|---|
| | (PDB: 6WMK) | (PDB: 7MWQ) | (PDB: 7MWR) |
| Data Collection | | | |
| Space group | P 2₁ | P1 | P 2₁2₁2₁ |
| Cell dimensions | | | |
| a, b, c (Å) | 56.07, 38.17, 60.37 | 61.31, 73.45, 4.14 | 45.40, 99.77, 122.09 |
| α, β, γ (°) | 90, 98.26, 90 | 108.39, 106.70, 110.15 | 90.0, 90.0, 90.0 |
| Resolution (Å) | 38.03-2.20 | 51.5-2.56 | 42.56-2.2 |
| | (2.42-2.20) | (2.65-2.56) | (2.27-2.20) |
| R_{merge} (%) | 7 (56.9) | 8.3 (82.8) | 3.1 (49.2) |
| Rₚᵢₘ (%) | 4.6 (36.5) | 6.6 (69.5) | 3.1 (49.2) |
| I/σ(I) | 6.3 (1.4) | 4.7 (1.07) | 15.9 (1.6) |
| CC_{1/2} | 0.995 (0.705) | 0.991(0.651) | 0.999 (0.757) |
| Completeness (%) | 94.2 (99.2) | 97.9 (93.4) | 99.8 (99.0) |
| Redundancy | 3.3 (3.3) | 2.3 (2.4) | 2.0 (2.0) |
| Refinement | | | |
| Resolution (Å) | 38.03-2.20 | 51.56-2.56 | 42.56-2.2 |
| | (2.42-2.20) | (2.65-2.56) | (2.27-2.20) |
| No. reflections | 12330 | 32540 | 28939 |
| R_{work}/R_{free} (%) | 25.3/28.3 | 23.2/26.9 | 21.1/25.2 |
| | (29.9/37.1) | (36.9/41.9) | (40.6/40.1) |
| No. atoms | 2154 | 6384 | 3514 |
| Protein | 2105 | 6370 | 11544 |
| Ligand | n/a | n/a | 7 |
| Water | 49 | 14 | 82 |
| Ramachandran | 96.80/3.20 | 98.64/1.11 | 97.77/2.23 |
| Favored/allowed | | 0.25 | 0.00 |
| Outlier (%) | | | |
| R.m.s. deviations | | | |
| Bond lengths (Å) | 0.002 | 0.002 | 0.002 |
| Bond angles (°) | 0.394 | 0.40 | 0.41 |
| B_{factors} (Å²) | | | |
| Protein | 55.00 | 75.64 | 52.36 |
| Ligand | n/a | n/a | 78.04 |
| Water | 42.13 | 53.18 | 53.31 |

Data were collected from one crystal per condition.
^{a}Values given in parentheses refer to reflections in the outer resolution shell. For calculation of R_{free}, 5% of all reflections were omitted from refinement.

We also determined the structure of a class two design, LHD101A53/B4 (2.2 Å), in which protomer A is fused to DHR53 and B to DHR4 (FIG. 2B and table 11). The crystal structure is again very close to the design model at both the interface and fusion junction, as well as the repeat protein region. In class two designs, the interface beta strand pair is reinforced by flanking helices that, unlike class one designs, are in direct contact with both each other and the interface beta sheet. The solvent exposed side of the beta interface consists primarily of electrostatic interactions (FIG. 2C, purple box). The buried side of the beta interface consists of exclusively hydrophobic side chains. Together with apolar side chains on the flanking helices of both protomers, these residues form a closely packed core interface (FIG. 2C, brown box) that is further stabilized by solvent exposed polar interactions between the flanking helices. Notably, the designed semi-buried polar interaction network centered on Tyr173 is maintained in the crystal structure (FIG. 2C, gray box).

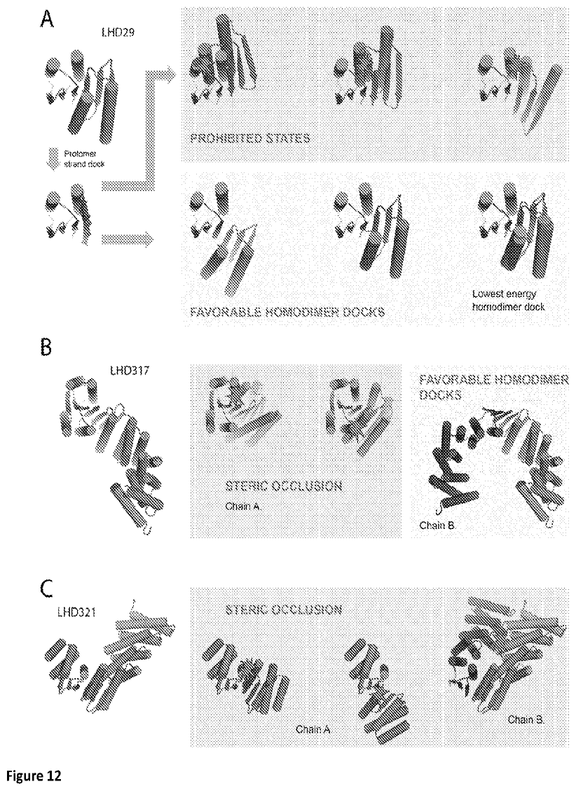

As described above, the third of our implicit negative design principles for avoiding unwanted self association was to incorporate structural elements incompatible with beta sheet extension in homo-dimeric species (FIG. 1D). To assess the utility of this principle, we took advantage of the limited number of possible off target edgestrand interactions that can form (FIG. 1C), and docked all protomers against themselves on the edge strand that participates in the heterodimer interface and calculated the Rosetta™ binding energy after relaxing of the resulting homodimeric dock (FIG. 12A). Homodimer docks of the protomers that chromatographed as monomers in SEC had unfavorable energies compared to those that showed evidence of self association in agreement with our initial hypothesis (FIG. 1D), and visual inspection of these docks suggested that homodimerization was likely prevented by the presence of sterically blocking secondary structure elements (FIG. 12).

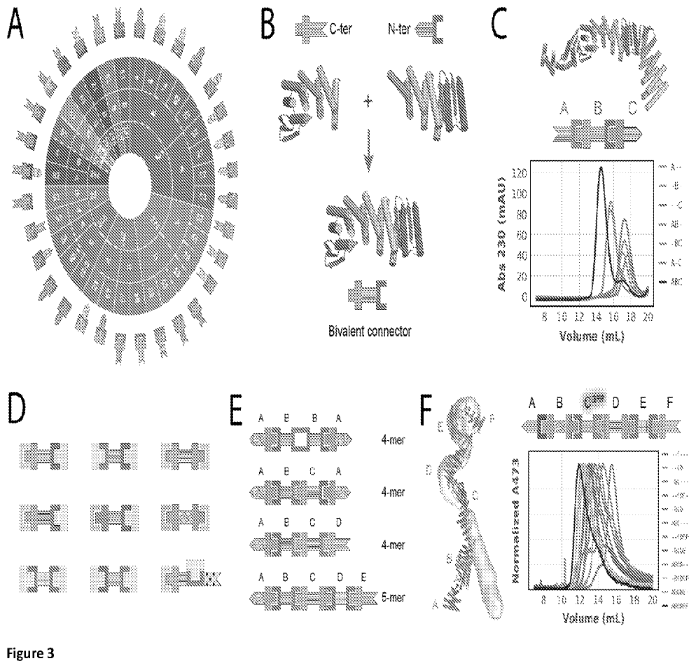

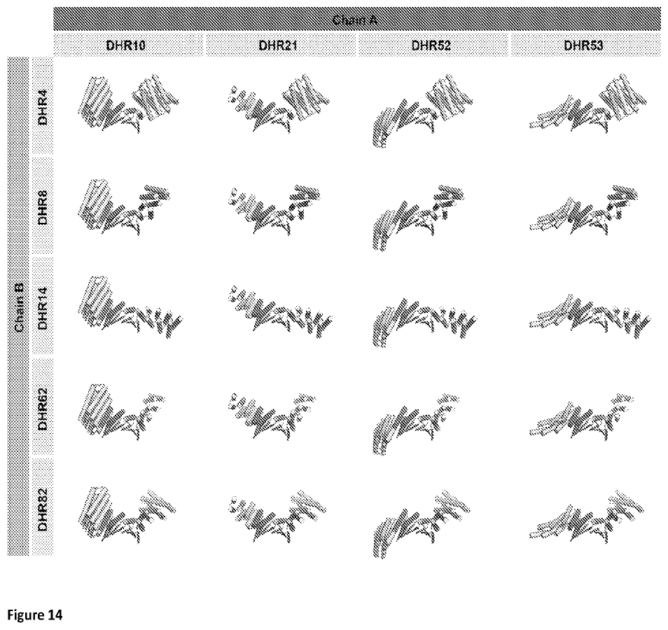

In addition to the crystallized fusion proteins (FIG. 2B), 28 more experimentally verified rigid fusion proteins were generated using the 11 base heterodimers and LHD274 (FIG. 3A). The DHR fusions retained both the oligomeric state and binding activity of the unfused counterparts, demonstrating that the designed heterodimers are robust to fusion (FIG. 8E, 11E, 13). With these fusions, there are 74 different possible heterodimeric complexes each with unique molecular scaffolding shapes. The majority of the fusions involve protomers of LHD274 and LHD101. Fusions to LHD101 protomers alone already enable the formation of 30 distinct heterodimeric complexes (FIG. 14).

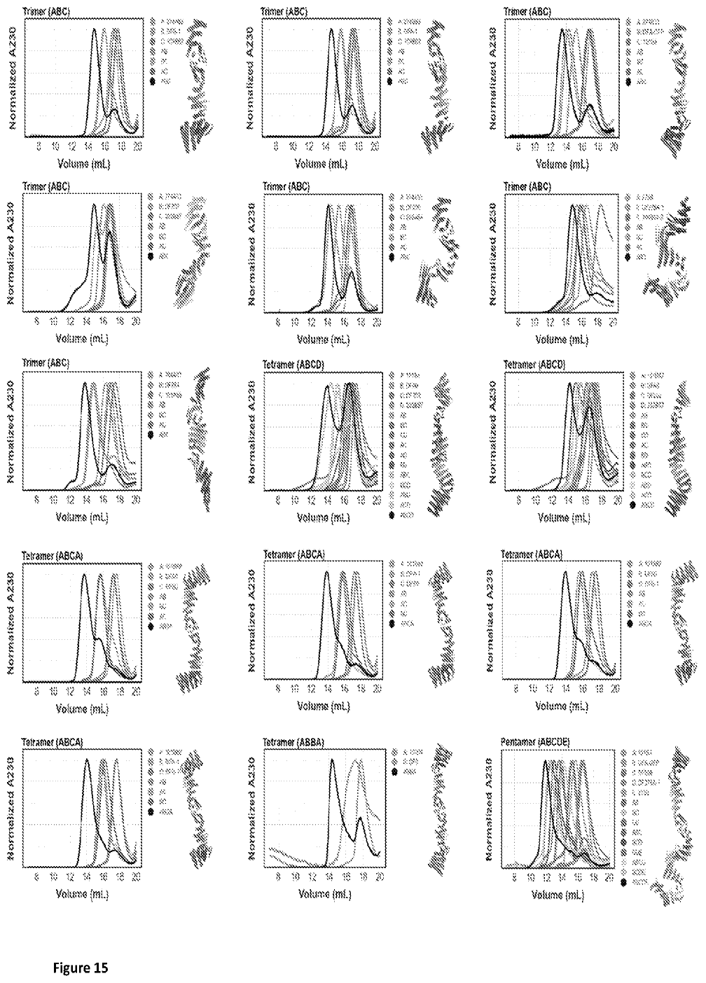

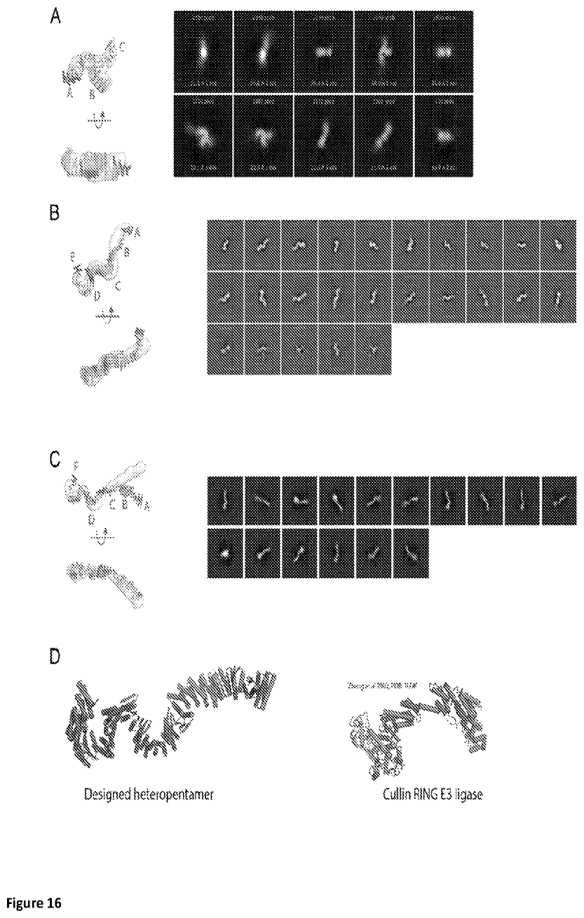

Larger multicomponent hetero-oligomeric protein assemblies require subunits that can interact with more than one binding partner at the same time. To this end, we generated single chain bivalent linear connector proteins. We searched for two protomers of different heterodimers that 1) share the same DHR as fusion partner and 2) have compatible termini. Designs fulfilling these criteria can be simply spliced together into a single protein chain on overlapping DHR repeats in a design-free fashion (FIG. 3B). Mixing a linear connector (“B”) with its two cognate binding partners (“A” and “C”) yields a linearly arranged heterotrimer (“ABC”) in which the two terminal capping components A and C are connected through component B, but otherwise are not in direct contact with each other (FIG. 3C). We analyzed the assembly of this heterotrimer and all possible controls by SEC (FIG. 3C), and observed stepwise assembly of the ABC heterotrimer with clear baseline separation from AB and BC heterodimers, as well as from monomeric components (FIG. 3C). Using the 9 different linear connectors created using the above described modular splicing approach (FIG. 3D), we in total assembled 20 heterotrimers including a complex verified by negative-stain electron microscopy (nsEM) (FIGS. 15 and 16A).

Linearly arranged hetero-oligomers beyond trimers contain more than one connector subunit in tandem per assembly in contrast to the single connector in heterotrimers. We successfully assembled ABCA and ABCD heterotetramers, each containing two different linear connectors (B and C) and either one or two terminal caps (2×A, or A+D), an ABBA heterotetramer using a homodimeric central connector (2×B) and one terminal cap (2×A), and a negative stain EM verified heteropentamer (ABCDE) containing 3 unique linear connectors and two caps (FIG. 3E, FIGS. 15 and 16B). We followed the assembly of an ABCDEF hetero-hexamer in SEC by GFP-tagging one of the components and monitoring GFP absorbance. The full assembly as well as sub-assemblies generated as controls eluted as monodisperse peaks, with elution volumes agreeing well with expected assembly sizes (FIG. 3F). Negative stain EM reconstruction of the hexamer confirmed all components were present (FIGS. 3F and 16C). Deviation of the experimentally observed shape from the design model likely arises from small inaccuracies in one of the components that cause a lever-arm effect (FIG. 2B).

The design-free generation of bivalent connector proteins from the DHR fusions facilitates the assembly of considerable diversity of asymmetric hetero oligomers. We modularly combined these connectors with each other and with monovalent terminal caps to create 36 hetero-oligomers with up to 6 unique chains which we experimentally validated by SEC and electron microscopy. This number can be readily increased to 489 by including all available components (FIG. 3A,D and supplementary spreadsheet). Since all fusions are rigid helical fusions, the overall molecular shapes of the complexes are well defined allowing control over the spatial arrangement of individual components which could be useful for scaffolding and other applications. Our linear assemblies resemble elongated modular multi-protein complexes found in nature (FIG. 16D), like the Cullin RING E3 Ligases 28 that mediate ubiquitin transfer by geometrically orienting the target protein and catalytic domain.

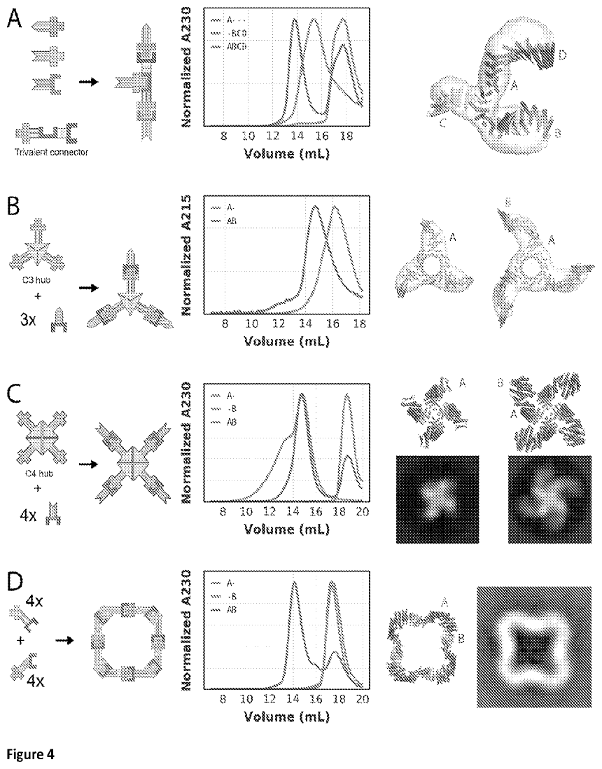

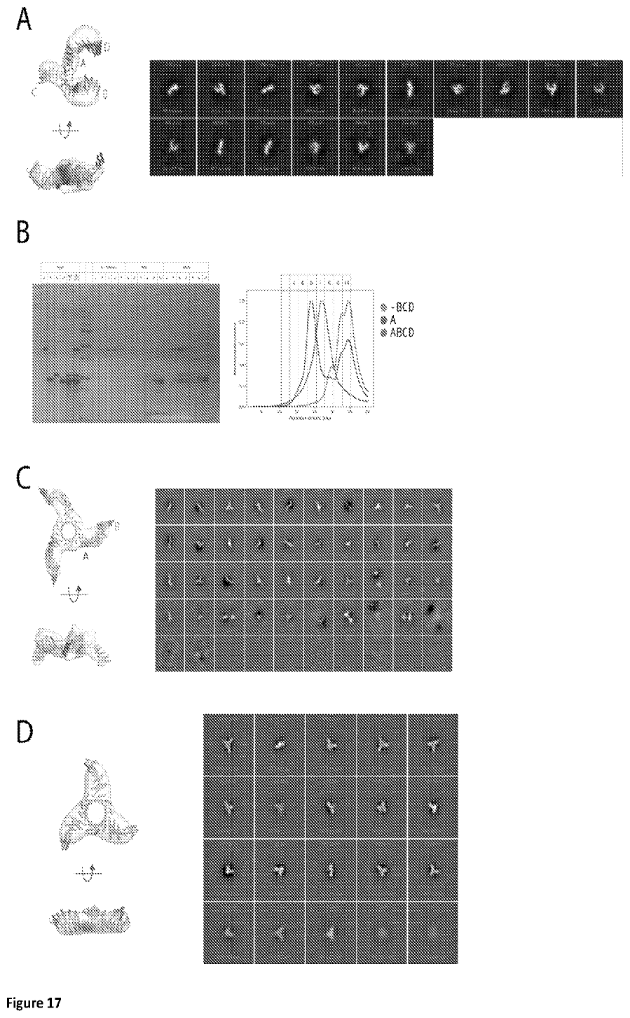

We next sought to go beyond the linear assemblies described thus far and build branched and closed assemblies. Trivalent connectors can be generated from heterodimers in which one protomer has both N- and C-terminal helices (LHD275A, LHD278A, LHD289A, LHD317A). Such protomers can be fused to two helical repeat proteins and spliced together with different halves of other heterodimer protomers via a common DHR repeat (FIGS. 3A,B and 4A). The resulting branched connectors (“A”) are capable of binding the three cognate binding partners (“B”,“C”,“D”) simultaneously and conceptually resemble Ste5 and related scaffolding proteins that organize MAP kinase signal transduction pathways in eukaryotes (29). Through SEC analyses we verified the assembly of two different tetrameric branched ABCD complexes, each containing one trivalent branched connector bound to three terminal caps (FIGS. 4B and 17A,B). For one of these, the complex was confirmed by negative stain EM class averages and 3D reconstructions indicating not only that all binding partners are present, but also that the shape closely matches the designed model (FIGS. 4A and 17A).

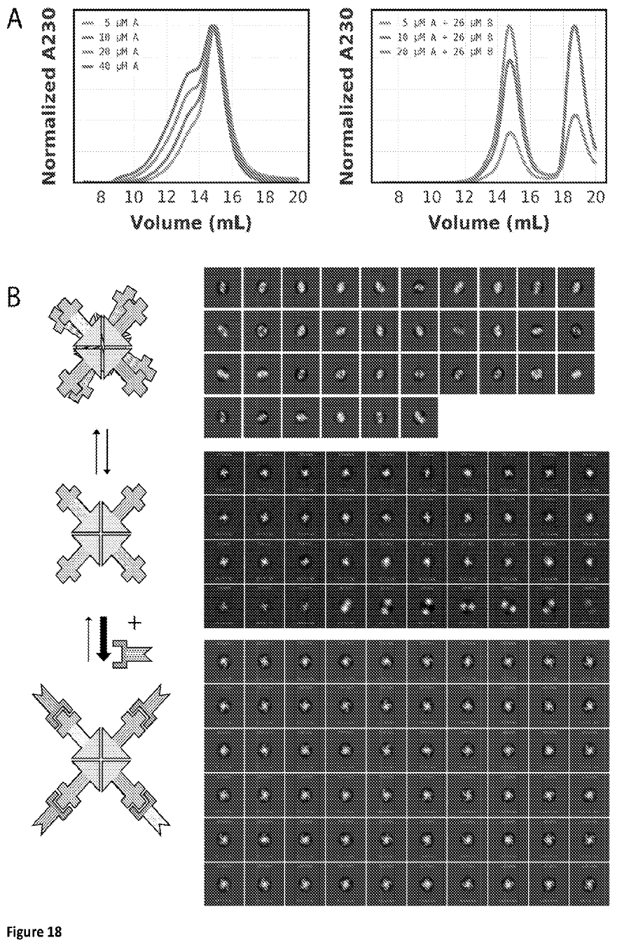

A different type of branched assemblies are “star shaped” oligomers with cyclic symmetries, akin to natural assemblies formed by IgM and the Inflammasome. Using the design-free alignment approach described above (FIG. 3B), we fused our new building blocks (FIG. 3A) to previously designed homo-oligomers, that terminate in helical repeat proteins (FIG. 4B,C). Such fusions yield central homo oligomeric hubs (“A_n”) that can bind multiple copies of the same binding partner (“n*B”). We generated C3- and C4-symmetric “hubs” that can bind 3 or 4 copies of their binding partners, respectively (FIG. 4B,C). In both cases, the oligomeric hubs are stable and soluble in isolation and readily form the target complexes when mixed with their binding partners, as confirmed by SEC chromatography, negative stain EM class averages and 3D reconstructions (FIG. 4B,C and FIG. 17C, 18). For the C4-symmetric hub in the absence of its binding partner we observed an additional concentration-dependent peak on SEC (FIG. 4C, FIG. 18A), indicating formation of a higher-order complex. This is likely a dimer of C4 hubs, since the C4 hub contains the redesigned protomer LHD274B, that despite its reduced homodimerization propensity compared to parent design LHD29B still weakly homodimerizes (FIG. 10). Notably, addition of the binding partner disrupted the higher order assembly, yielding the on-target octameric (A4B4) complex (FIG. 4C), illustrating this system can reconfigure.

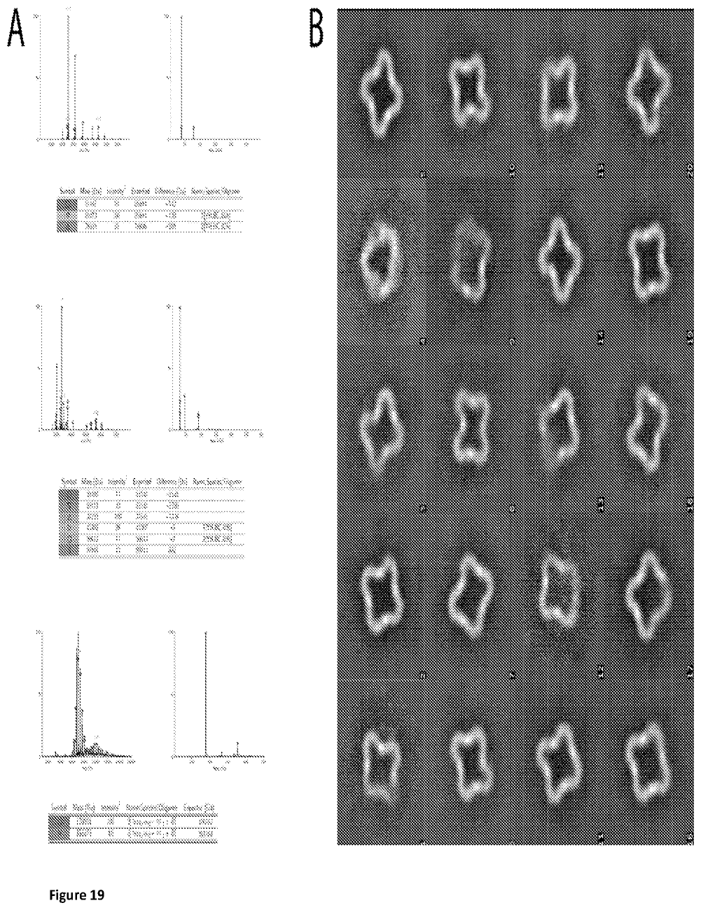

In addition to linear and branched assemblies, we designed closed symmetric two-component assemblies. Designing these presents a more complex geometric challenge, as the interaction geometry of all pairs of subunits must be compatible with a single closed three dimensional structure of the entire assembly. We used architecture-aware rigid helical fusion (7, 33) to generate two bivalent connector proteins from the crystal-verified fusions of LHD29 and LD101 (FIG. 2B) that allow assembly of a perfectly closed C4-symmetric hetero-oligomeric two-component ring (FIG. 4D). Individually expressed and purified components are stable and soluble monomers in isolation, as confirmed by SEC and native MS (FIG. 4D, FIG. 19). Upon mixing, the components form a higher-order complex that by native MS comprises four copies of each component. Negative stain EM confirms that this higher-order complex is nearly identical to the designed C4 symmetric ring (FIG. 4D, FIG. 19). Using our heterodimeric building blocks, the same architecture-aware fusion method can be used to design a variety of different closed symmetric complexes that assemble from well-behaved components.

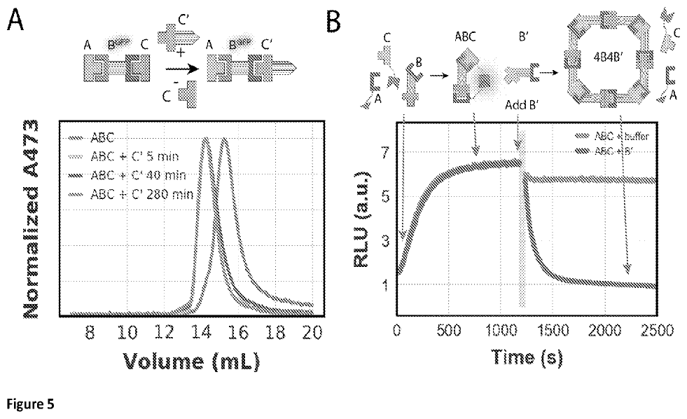

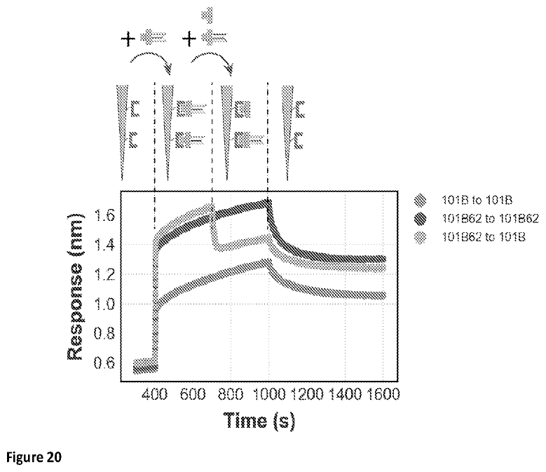

Because our designed building blocks are stable in solution and not kinetically trapped in off-target homo-oligomeric states, the assemblies they form can rapidly reconfigure, as outlined in FIG. 1A and observed for the C4-symmetric hub shown in FIG. 4C. We further evaluated this reconfigurability using two different approaches to assemble and then reconfigure a heterotrimer. First, we assembled an ABC trimer using a GFP-tagged version of a linear connector B and unfused terminal caps A and B (FIG. 5A). The pre-incubated trimer was next mixed with either buffer or a DHR fusion variant of component C, called C′. As indicated by the shift of the trimer peak in SEC, component C (8.6 kDa) readily exchanged with C′ (27.7 kDa), to form a larger ABC′ complex. Subunit exchange was confirmed by biolayer interferometry (FIG. 20).

Second, we followed the transition, through subunit exchange, of a linear heterotrimer to the designed C4 symmetric hetero-oligomeric two-component ring using an in vitro split luciferase reporter assay (FIG. 5B). We first assembled an ABC heterotrimer, in which chain B is one of the two components of the ring, and A and C are the corresponding terminal cap binding partners fused to the two parts of the split luciferase. In absence of B, components A and C do not interact. Upon addition of B, the heterotrimer forms, resulting in luciferase activity. Subsequent addition of the second component of the C4 symmetric ring, B′, led to a rapid decrease in luciferase activity, indicating disassembly of the trimer (FIG. 5B) consistent with ring formation from the two components observed in SEC (FIG. 4C). Taken together, these experiments indicate that subunit exchange can take place on the several minute time scale and pave the way for applications that require designed dynamic reconfigurability of multiprotein complexes.

Using site-saturated mutagenesis (SSM) we generated point mutants of LHD101A that show stronger binding to LHD101B (and thus also to fusions of LHD101B) than the original LHD101A sequence. In particular, we found that dissociation was much slower for the point mutants than for the original LHD101A sequence, while association rates remain mostly unchanged.

>LHD101A Q42M
(SEQ ID NO: 2)
GRQEKVLKSIEETVRKMGVTMETHRSGNEVKVVIKGLH
IKQMRQLYRDVRETSKKQGVETEIEVEGDTVTIVVRE
>LHD101A R43V
(SEQ ID NO: 3)
GRQEKVLKSIEETVRKMGVTMETHRSGNEVKVVIKGLHI
KQQVQLYRDVRETSKKQGVETEIEVEGDTVTIVVRE
>LHD101A V69A
(SEQ ID NO: 4)
GRQEKVLKSIEETVRKMGVTMETHRSGNEVKVVIKGLHI
KQQRQLYRDVRETSKKQGVETEIEVEGDTQTIVVRE
>LHD101A T70W
(SEQ ID NO: 4)
GRQEKVLKSIEETVRKMGVTMETHRSGNEVKVVIKGLHI
KQQRQLYRDVRETSKKQGVETEIEVEGDTVWIVVRE

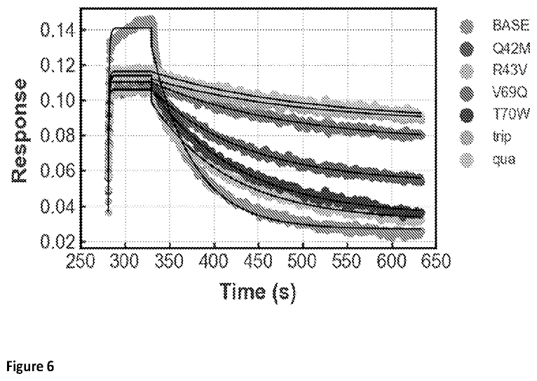

These are point mutants of LHD101A (mutant numbering e.g. Q42M is for the basic LHD101A binding domain, can be different in the fusions) that bind stronger to LHD101B and all fusion variants of LHD101B. See FIG. 6 and Table 12.

TABLE 12
Dissociation rate constants become slower
in mutants compared to base design (101Awt)

| Sample ID | kdis(1/s) | kdis Error |
|---|---|---|
| 101Awt | 2.14E−02 | 2.94E−04 |
| Q42M | 9.41E−03 | 1.48E−04 |
| R43V | 1.13E−02 | 1.71E−04 |
| V69Q | 6.53E−03 | 1.92E−04 |
| T70W | 1.02E−02 | 1.54E−04 |
| triple | 5.89E−03 | 3.01E−04 |
| qua | 5.79E−03 | 2.56E−04 |

Our implicit negative design principles enable the de novo design of heterodimer pairs for which the individual protomers are stable in solution and readily form their target heterodimeric complexes upon mixing. Rigid fusion of multiple halves of heterodimers onto DHR proteins enables the design of higher order asymmetric multiprotein complexes that range in shape from linear and cyclic to branched. The large number of characterized rigid fusions with different shapes and the modular nature of our assembly platform enables fine tuning of protein complex geometries, for example by changing the number of repeats in the DHR proteins and using the same heterodimer half fused to different DHRs.

Since the unfused protomers are small (between 7 and 15 kDa without DHR or tags), they can be readily fused to target proteins of interest. Our bivalent or trivalent connectors can then be used to colocalize and geometrically position two or three such target protein fusions, respectively, and our symmetric hubs can be used to colocalize and position multiple copies of the same target fusion. Due to the modularity of our system, the same set of target fusions can be arranged in multiple different arrangements with adjustable distances, angles, and copy numbers by simply using different connectors. Since all components are soluble and well-behaved in isolation, stepwise assembly schemes are possible in which, for example, two constitutively expressed target protein fusions do not interact until expression of a connector is induced, leading to formation of a trimeric complex. Using one of our ABCD tetramers, such a system can be extended to enable simple logic operations: two target proteins fused to components A and D will only be colocalized if both B and C are present. Since the thermodynamic and kinetic properties of our heterodimers are not altered by rigid fusions, the behaviour of multi-component assemblies can be predicted based on the properties of the individual interfaces (compare FIG. 11F,G). Our designed assemblies can reconfigure by addition of new subunits and loss of already incorporated ones, opening the door to a wide range of new applications for de novo protein design.

### REFERENCES AND NOTES

1. S. E. Tusk, N. J. Delalez, R. M. Berry, Subunit Exchange in Protein Complexes. J. Mol. Biol. 430, 4557-4579 (2018).
2. C. Engel, S. Neyer, P. Cramer, Distinct Mechanisms of Transcription Initiation by RNA Polymerases I and II. Annu. Rev. Biophys. 47, 425-446 (2018).
3. P. M. J. Burgers, T. A. Kunkel, Eukaryotic DNA Replication Fork. Annu. Rev. Biochem. 86, 417-438 (2017).
4. S. Gonen, F. DiMaio, T. Gonen, D. Baker, Design of ordered two-dimensional arrays mediated by noncovalent protein-protein interfaces. Science. 348, 1365-1368 (2015).
5. Y. Hsia, J. B. Bale, S. Gonen, D. Shi, W. Sheffler, K. K. Fong, U. Nattermann, C. Xu, P.-S. Huang, R. Ravichandran, S. Yi, T. N. Davis, T. Gonen, N. P. King, D. Baker, Design of a hyperstable 60-subunit protein dodecahedron. [corrected]. Nature. 535, 136-139 (2016).
6. N. P. King, J. B. Bale, W. Sheffler, D. E. McNamara, S. Gonen, T. Gonen, T. O. Yeates, D. Baker, Accurate design of co-assembling multi-component protein nanomaterials. Nature. 510, 103-108 (2014).
7. Y. Hsia, R. Mout, W. Sheffler, N. I. Edman, I. Vulovic, Y.-J. Park, R. L. Redler, M. J. Bick, A. K. Bera, A. Courbet, A. Kang, T. J. Brunette, U. Nattermann, E. Tsai, A. Saleem, C. M. Chow, D. Ekiert, G. Bhabha, D. Veesler, D. Baker, Design of multi-scale protein complexes by hierarchical building block fusion. Nat. Commun. 12, 2294 (2021).
8. A. J. Ben-Sasson, J. L. Watson, W. Sheffler, M. C. Johnson, A. Bittleston, L. Somasundaram, J. Decarreau, F. Jiao, J. Chen, I. Mela, A. A. Drabek, S. M. Jarrett, S. C. Blacklow, C. F. Kaminski, G. L. Hura, J. J. De Yoreo, J. M. Kollman, H. Ruohola-Baker, E. Derivery, D. Baker, Design of biologically active binary protein 2D materials. Nature. 589, 468-473 (2021).
9. R. Divine, H. V. Dang, G. Ueda, J. A. Fallas, I. Vulovic, W. Sheffler, S. Saini, Y. T. Zhao, I. X. Raj, P. A. Morawski, M. F. Jennewein, L. J. Homad, Y.-H. Wan, M. R. Tooley, F. Seeger, A. Etemadi. M. L. Fahning, J. Lazarovits, A. Roederer, A. C. Walls, L. Stewart, M. Mazloomi, N. P. King, D. J. Campbell, A. T. McGuire, L. Stamatatos, H. Ruohola-Baker. J. Mathieu, D. Veesler, D. Baker, Designed proteins assemble antibodies into modular nanocages. Science. 372 (2021), doi:10.1126/science.abd9994.
10. Z. Chen. S. E. Boyken, M. Jia, F. Busch, D. Flores-Solis, M. J. Bick, P. Lu, Z. L. VanAernum, A. Sahasrabuddhe, R. A. Langan, S. Bermeo, T. J. Brunette, V. K. Mulligan, L. P. Carter, F. DiMaio, N. G. Sgourakis, V. H. Wysocki, D. Baker, Programmable design of orthogonal protein heterodimers. Nature. 565, 106-111 (2019).
11. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science. 352, 680-687 (2016).
12. Z. Chen, R. D. Kibler, A. Hunt, F. Busch, J. Pearl, M. Jia, Z. L. VanAernum, B. I. M. Wicky, G. Dods, H. Liao, M. S. Wilken, C. Ciarlo, S. Green, H. El-Samad, J. Stamatoyannopoulos, V. H. Wysocki, M. C. Jewett, S. E. Boyken, D. Baker, De novo design of protein logic gates. Science. 368, 78-84 (2020).
13. H. Gradišar, R. Jerala, De novo design of orthogonal peptide pairs forming parallel coiled-coil heterodimers. J. Pept. Sci. 17, 100-106 (2011).
14. C. L. Edgell, A. J. Smith, J. L. Beesley, N. J. Savery, D. N. Woolfson, De Novo Designed Protein-Interaction Modules for In-Cell Applications. ACS Synth. Biol. 9, 427-436 (2020).
15. A. Leaver-Fay, R. Jacak, P. B. Stranges, B. Kuhlman, A generic program for multistate protein design. PLoS One. 6, e20937 (2011).
16. A. Leaver-Fay, K. J. Froning, S. Atwell, H. Aldaz, A. Pustilnik, F. Lu, F. Huang, R. Yuan, S. Hassanali, A. K. Chamberlain, J. R. Fitchett, S. J. Demarest, B. Kuhlman, Computationally Designed Bispecific Antibodies using Negative State Repertoires. Structure, 24, 641-651 (2016).
17. S. J. Fleishman, D. Baker, Role of the biomolecular energy gap in protein design, structure, and evolution. Cell. 149, 262-273 (2012).
18. D. D. Sahtoe, A. Coscia, N. Mustafaoglu, L. M. Miller, D. Olal, I. Vulovic, T.-Y. Yu, I. Goreshnik, Y.-R. Lin, L. Clark, F. Busch, L. Stewart, V. H. Wysocki, D. E. Ingber, J. Abraham, D. Baker, Transferrin receptor targeting by de novo sheet extension. Proc. Natl. Acad. Sci. U.S.A. 118 (2021), doi:10.1073/pnas.2021569118.
19. P. B. Stranges, M. Machius, M. J. Miley, A. Tripathy, B. Kuhlman, Computational design of a symmetric homodimer using β-strand assembly. Proc. Natl. Acad. Sci. U.S.A 108, 20562-20567 (2011).
20. H. Remaut, G. Waksman, Protein-protein interaction through beta-strand addition. Trends Biochem. Sci. 31, 436-444 (2006).
21. B. Koepnick, J. Flatten, T. Husain, A. Ford, D.-A. Silva, M. J. Bick, A. Bauer, G. Liu, Y. Ishida, A. Boykov, R. D. Estep, S. Kleinfelter, T. Nørgård-Solano, L. Wei, F. Players, G. T. Montelione, F. DiMaio, Z. Popović, F. Khatib, S. Cooper, D. Baker, De novo protein design by citizen scientists. Nature. 570, 390-394 (2019).
22. T. J. Brunette, M. J. Bick, J. M. Hansen, C. M. Chow, J. M. Kollman, D. Baker, Modular repeat protein sculpting using rigid helical junctions. Proc. Natl. Acad. Sci. U.S.A 117, 8870-8875 (2020).
23. Y.-R. Lin, N. Koga, R. Tatsumi-Koga, G. Liu, A. F. Clouser, G. T. Montelione, D. Baker, Control over overall shape and size in de novo designed proteins. Proc. Natl. Acad. Sci. U S. A. 112, E5478-85 (2015).
24. N. Koga, R. Tatsumi-Koga, G. Liu, R. Xiao, T. B. Acton, G. T. Montelione, D. Baker, Principles for designing ideal protein structures. Nature. 491, 222-227 (2012).
25. J. K. Leman, B. D. Weitzner, S. M. Lewis, J. Adolf-Bryfogle, N. Alam, R. F. Alford, M. Aprahamian, D. Baker, K. A. Barlow, P. Barth, B. Basanta, B. J. Bender, K. Blacklock, J. Bonet, S. E. Boyken, P. Bradley, C. Bystroff, P. Conway, S. Cooper, B. E. Correia, B. Coventry, R. Das, R. M. De Jong, F. DiMaio, L. Dsilva, R. Dunbrack, A. S. Ford, B. Frenz, D. Y. Fu, C. Geniesse, L. Goldschmidt, R. Gowthaman, J. J. Gray, D. Gront, S. Guffy, S. Horowitz, P.-S. Huang, T. Huber, T. M. Jacobs, J. R. Jeliazkov, D. K. Johnson, K. Kappel, J. Karanicolas, H. Khakzad, K. R. Khar, S. D. Khare. F. Khatib, A. Khramushin, I. C. King, R. Kleffner, B. Koepnick, T. Kortemme, G. Kuenze, B. Kuhlman, D. Kuroda, J. W. Labonte, J. K. Lai, G. Lapidoth, A. Leaver-Fay, S. Lindert, T. Linsky, N. London, J. H. Lubin, S. Lyskov, J. Maguire, L. Malmström, E. Marcos, O. Marcu, N. A. Marze, J. Meiler, R. Moretti, V. K. Mulligan, S. Nerli, C. Norn, S. Ó'Conchúir, N. Ollikainen, S. Ovchinnikov, M. S. Pacella, X. Pan, H. Park, R. E. Pavlovicz, M. Pethe, B. G. Pierce, K. B. Pilla, B. Raveh, P. D. Renfrew, S. S. R. Burman, A. Rubenstein, M. F. Sauer, A. Scheck, W. Schief, O. Schueler-Furman, Y. Sedan, A. M. Sevy, N. G. Sgourakis, L. Shi, J. B. Siegel, D.-A. Silva, S. Smith, Y. Song, A. Stein, M. Szegedy, F. D. Teets, S. B. Thyme, R. Y.-R. Wang, A. Watkins, L. Zimmerman, R. Bonneau, Macromolecular modeling and design in Rosetta: recent methods and frameworks. Nat. Methods. 17, 665-680 (2020).
26. B. Coventry, D. Baker, Protein sequence optimization with a pairwise decomposable penalty for buried unsatisfied hydrogen bonds. Cold Spring Harbor Laboratory (2020), p. 2020.06.17.156646.
27. T. J. Brunette, F. Parmeggiani, P.-S. Huang, G. Bhabha, D. C. Ekiert, S. E. Tsutakawa, G. L. Hura, J. A. Tainer, D. Baker, Exploring the repeat protein universe through computational protein design. Nature. 528, 580-584 (2015).
28. J. R. Lydeard, B. A. Schulman, J. W. Harper, Building and remodelling Cullin-RING E3 ubiquitin ligases. EMBO Rep. 14, 1050-1061 (2013).
29. L. K. Langeberg, J. D. Scott, Signalling scaffolds and local organization of cellular behaviour. Nat. Rev. Mol. Cell Biol. 16, 232-244 (2015).
30. H. W. Schroeder Jr, L. Cavacini, Structure and function of immunoglobulins. J. Allergy Clin. Immunol. 125, S41-52 (2010).
31. P. Broz, V. M. Dixit, Inflammasomes: mechanism of assembly, regulation and signalling. Nat. Rev. Immunol. 16, 407-420 (2016).
32. L. Doyle, J. Hallinan, J. Bolduc, F. Parmeggiani, D. Baker, B. L. Stoddard, P. Bradley, Rational design of α-helical tandem repeat proteins with closed architectures. Nature. 528, 585-588 (2015).
33. I. Vulovic, Q. Yao, Y.-J. Park, A. Courbet, A. Norris, F. Busch, A. Sahasrabuddhe, H. Merten, D. D. Sahtoe, G. Ueda, J. A. Fallas, S. J. Weaver, Y. Hsia, R. A. Langan, A. Plückthun, V. H. Wysocki, D. Veesler, G. J. Jensen, D. Baker, Generation of ordered protein assemblies using rigid three-body fusion. Cold Spring Harbor Laboratory (2020), p. 2020.07.18.210294.
34. F. Khatib, S. Cooper, M. D. Tyka, K. Xu, I. Makedon, Z. Popovic, D. Baker, F. Players, Algorithm discovery by protein folding game players. Proc. Natl. Acad. Sci. U S. A. 108, 18949-18953 (2011).
35. A. Chevalier, D.-A. Silva, G. J. Rocklin, D. R. Hicks, R. Vergara, P. Murapa, S. M. Bernard, L. Zhang, K.-H. Lam, G. Yao, C. D. Bahl, S.-I. Miyashita, I. Goreshnik, J. T. Fuller, M. T. Koday, C. M. Jenkins, T. Colvin, L. Carter, A. Bohn, C. M. Bryan, D. A. Fernández-Velasco, L. Stewart, M. Dong, X. Huang, R. Jin, I. A. Wilson, D. H. Fuller, D. Baker, Massively parallel de novo protein design for targeted therapeutics. Nature. 550, 74-79 (2017).
36. P. Hosseinzadeh, G. Bhardwaj, V. K. Mulligan, M. D. Shortridge, T. W. Craven, F. Pardo-Avila, S. A. Rettie, D. E. Kim, D.-A. Silva, Y. M. Ibrahim, I. K. Webb, J. R. Cort, J. N. Adkins, G. Varani, D. Baker, Comprehensive computational design of ordered peptide macrocycles. Science. 358, 1461-1466 (2017).
37. B. Dang, H. Wu, V. K. Mulligan, M. Mravic, Y. Wu, T. Lemmin, A. Ford, D.-A. Silva, D. Baker, W. F. DeGrado, De novo design of covalently constrained mesosize protein scaffolds with unique tertiary structures. Proc. Natl. Acad. Sci. U.S.A 114, 10852-10857 (2017).
38. S. J. Fleishman, A. Leaver-Fay, J. E. Corn, E.-M. Strauch, S. D. Khare, N. Koga, J. Ashworth, P. Murphy, F. Richter, G. Lemmon, J. Meiler, D. Baker, RosettaScripts: a scripting language interface to the Rosetta macromolecular modeling suite. PLoS One. 6, e20161 (2011).
39. G. Bhardwaj, V. K. Mulligan, C. D. Bahl, J. M. Gilmore, P. J. Harvey, O. Cheneval, G. W. Buchko, S. V. S. R. K. Pulavarti, Q. Kaas, A. Eletsky, P.-S. Huang, W. A. Johnsen, P. J. Greisen, G. J. Rocklin, Y. Song, T. W. Linsky, A. Watkins, S. A. Rettie, X. Xu, L. P. Carter, R. Bonneau, J. M. Olson, E. Coutsias, C. E. Correnti, T. Szyperski, D. J. Craik, D. Baker, Accurate de novo design of hyperstable constrained peptides. Nature. 538, 329-335 (2016).
40. R. F. Alford, A. Leaver-Fay, J. R. Jeliazkov, M. J. O'Meara, F. P. DiMaio, H. Park, M. V. Shapovalov, P. D. Renfrew, V. K. Mulligan, K. Kappel, J. W. Labonte, M. S. Pacella, R. Bonneau, P. Bradley, R. L. Dunbrack Jr, R. Das, D. Baker, B. Kuhlman, T. Kortemme, J. J. Gray, The Rosetta All-Atom Energy Function for Macromolecular Modeling and Design. J. Chem. Theory Comput. 13, 3031-3048 (2017).
41. M. C. Lawrence, P. M. Colman, Shape complementarity at protein/protein interfaces. J. Mol. Biol. 234, 946-950 (1993).
42. B. Dang, M. Mravic, H. Hu, N. Schmidt, B. Mensa, W. F. DeGrado, SNAC-tag for sequence-specific chemical protein cleavage. Nat. Methods. 16, 319-322 (2019).
43. P. Virtanen, R. Gommers, T. E. Oliphant, M. Haberland, T. Reddy, D. Cournapeau, E. Burovski, P. Peterson, W. Weckesser, J. Bright, S. J. van der Walt, M. Brett, J. Wilson, K. J. Millman, N. Mayorov, A. R. J. Nelson, E. Jones, R. Kern, E. Larson, C. J. Carey, İ. Polat, Y. Feng, E. W. Moore, J. VanderPlas, D. Laxalde, J. Perktold, R. Cimrman, I. Henriksen, E. A. Quintero, C. R. Harris, A. M. Archibald, A. H. Ribeiro, F. Pedregosa, P. van Mulbregt, SciPy 1.0 Contributors, SciPy 1.0: fundamental algorithms for scientific computing in Python. Nat. Methods. 17, 261-272 (2020).
44. Z. L. VanAernum, F. Busch, B. J. Jones, M. Jia, Z. Chen, S. E. Boyken, A. Sahasrabuddhe, D. Baker, V. H. Wysocki, Rapid online buffer exchange for screening of proteins, protein complexes and cell lysates by native mass spectrometry. Nat. Protoc. 15, 1132-1157 (2020).
45. M. T. Marty, A. J. Baldwin, E. G. Marklund, G. K. A. Hochberg, J. L. P. Benesch, C. V. Robinson, Bayesian deconvolution of mass and ion mobility spectra: from binary interactions to polydisperse ensembles. Anal. Chem. 87, 4370-4376 (2015).
46. A. J. McCoy, R. W. Grosse-Kunstleve, P. D. Adams, M. D. Winn, L. C. Storoni, R. J. Read, Phaser crystallographic software. J. Appl. Crystallogr. 40, 658-674 (2007).
47. W. Kabsch, XDS. Acta Crystallogr. D Biol. Crystallogr. 66, 125-132 (2010).
48. Z. Otwinowski, W. Minor, in Methods in Enzymology (Academic Press, 1997), vol. 276, pp. 307-326.
49. M. D. Winn, C. C. Ballard, K. D. Cowtan, E. J. Dodson, P. Emsley, P. R. Evans, R. M. Keegan, E. B. Krissinel, A. G. W. Leslie, A. McCoy, S. J. McNicholas, G. N. Murshudov, N. S. Pannu, E. A. Potterton, H. R. Powell, R. J. Read, A. Vagin, K. S. Wilson, Overview of the CCP4 suite and current developments. Acta Crystallogr. D Biol. Crystallogr. 67, 235-242 (2011).
50. P. D. Adams, P. V. Afonine, G. Bunkóczi, V. B. Chen, I. W. Davis, N. Echols, J. J. Headd, L.-W. Hung, G. J. Kapral, R. W. Grosse-Kunstleve, Others, PHENIX: a comprehensive Python-based system for macromolecular structure solution. Acta Crystallogr. D Biol. Crystallogr. 66, 213-221 (2010).
51. G. N. Murshudov, A. A. Vagin, E. J. Dodson, Refinement of macromolecular structures by the maximum-likelihood method. Acta Crystallogr. D Biol. Crystallogr. 53, 240-255 (1997).
52. P. Emsley, K. Cowtan, Coot: model-building tools for molecular graphics. Acta Crystallogr. D Biol. Crystallogr. 60, 2126-2132 (2004).
53. B. L. Nannenga, M. G. Iadanza, B. S. Vollmar, T. Gonen, Overview of electron crystallography of membrane proteins: crystallization and screening strategies using negative stain electron microscopy. Curr. Protoc. Protein Sci. Chapter 17, Unit17.15 (2013).
54. T. Grant, A. Rohou, N. Grigorieff, cisTEM, user-friendly software for single-particle image processing. Elife. 7 (2018), doi:10.7554/eLife.35383.
55. A. Punjani, J. L. Rubinstein, D. J. Fleet, M. A. Brubaker, cryoSPARC: algorithms for rapid unsupervised cryo-EM structure determination. Nat. Methods. 14, 290-296 (2017).

### Materials and Methods

### Protein Design

### Docking Procedure

As scaffolds for generating edge-strand heterodimers we used mixed alpha/beta proteins designed by citizen scientist (21) and variants of the fold-it scaffolds that were either expanded with additional helices (see backbone generation methods), and/or fused to de novo helical repeat (DHR) proteins (27). Edgestrand docking was performed as described previously (18). Exposed edgestrands suitable for docking were identified by calculating the solvent accessible surface area of beta sheet backbone atoms in all the scaffolds used in the docking procedure. Next, the c-alpha atoms of each strand of short 2 stranded parallel and antiparallel beta sheet motifs were aligned to the exposed edge strand yielding an aligned clashing strand and free dock strand. After removal after the aligned clashing strand, the docked strand was trimmed at N and/or C terminus in order to remove potential clashes and subsequently minimized using Rosetta™ FastRelax (34) to optimize backbone to backbone hydrogen bonds. Docks failing a specified threshold value (typically −4 using ref2015) for the backbone hydrogen bond scoreterm in Rosetta™ (hbond_lr_bb) were discarded. The minimized docked strands were next geometrically matched to the scaffold library using the MotifGraftMover to create a docked protein-protein complex (35).

### Interface Design

The interface residues of the docked heterodimer complexes were optimized using Rosetta™ combinatorial sequence (36-39) design using “ref2015” “beta_nov16” or “beta_genpot” as scorefunctions (40). The interface polarity of the docked heterodimer complexes were fine tuned in several ways (see supplement for description of design xml's). First, the HBNetMover™ (11) was used to install explicit hydrogen bond networks containing at least 3 hydrogen bonds across the interface. Later design rounds consisted of two separate interface sequence optimization steps. First interface residues were optimized without compositional constraints yielding a substantial number of hydrophobic interactions in the interface. The best designs were subsequently selected and hydrophobic residue pairs with the lowest Rosetta™ energy interactions across the interface were stored as a seed hydrophobic interaction hotspot. In a second round, a polar interaction network was designed around the fixed hydrophobic hotspot interaction using compositional constraints that favor polar interactions (26). Designs were filtered on interface properties such as binding energy, buried surface area, shape complementarity, degree of packing, and presence of unsatisfied buried polar atoms. A final selection was made by visual inspection of models.

### Backbone Generation and Scaffold Design

De novo designed protein scaffolds created by fold-it players (21) were expanded with C-terminal polyvaline helices using blueprint based backbone generation (23, 24). The amino acid identities of the newly built helices and their surrounding region were optimized using Rosetta™ combinatorial sequence designs using a flexible backbone. The resulting models were folded in silico using Rosetta™ folding simulations and trajectories that converged to the designed model structure without off-target minima were selected for rigid fusion and heterodimer design.

### Design of Rigid Fusions

To generate rigid fusions of scaffolds or heterodimers to DHRs we adapted the HFuse pipeline (22), (7): Fusion junctions were designed using the Fastdesign™ mover allowing backbone movement, and additional filters were included to ensure sufficient contact between DHR and scaffold/heterodimer. When fusing to heterodimers, an additional filter was employed to prevent additional contacts between the DHR and the other protomer of the dimer. Bivalent connectors were generated by aligning two proteins that share the same DHR along their shared helical repeats, and subsequently splicing together the sequences. To build the C3-symmetric “hub”, we used a previously published 12×toroid crystal structure (32). The starting structure was relaxed, Z axis aligned, and cut into three C3 symmetric chains. Then the HFuse software (22), (7) was used to sample DHR fusions to the exposed helical C-termini, and the newly created interfaces were redesigned using Rosetta™Scripts. For the C4 symmetric hub, we used a previously published C4-symmetric homooligomer that already contain a n-terminal DHR. For both hubs, matching DHR fusions of heterodimer protomers we then used the same align and splice approach as for the bivalent connectors.

### Design of C4 Rings

Using the relaxed crystal structures of LHD29 and LHD101 fused to their respective DHRs, the WORMS software (7, 9, 33) was used to fuse the two hetero-dimers into cyclic symmetrical rings. As one construct has exposed N-termini and the other has exposed C-termini, they were able to be fused head to tail without introduction of further building blocks. Briefly, the first 3 repeats of each repeat protein was allowed to be sampled as fusion points to ensure that the heterodimer interface was not altered. Following fusion into cyclic structures, fixed backbone junction design was applied to the new fusion point using Rosetta™Scripts (38), optimizing for shape complementarity (41). One design from each symmetry: C3, C4, C5, and C6 were selected for experimental testing.

### Protein Expression and Purification

Synthetic genes encoding designed proteins and their variants were purchased from Genscript or Integrated DNA technologies (IDT). Bicistronic genes were ordered in pET29b with the first cistron being either without tag or with an N-terminal sfGFP tag followed by the intercistronic sequence TAAAGAAGGAGATATCATATG (SEQ ID NO: 192). The second cistron was tagged with a polyhistidine His6x tag at the C-terminus. Plasmids encoding the individual protomers were ordered in pET29b either with or without Avi-Tag, with an N-terminal polyhistidine His6x tag followed by a TEV cleavage site, N-terminal polyhistidine His6x tag followed by a snac cleavage site or C-terminal polyhistidine His6x tag preceded by a snac tag (see supplementary spreadsheet for detailed construct information). Proteins were expressed in BL21 LEMO E. coli cells by autoinduction using TBII media (Mpbio) supplemented with 50x5052, 20 mM MgSO4 and trace metal mix, or in almost TB media containing 12 g peptone and 24 g yeast extract per liter supplement with 50x5052, 20 mM MgSO4, trace metal mix and 10× phosphate buffer. Proteins were expressed under antibiotics selection at 37 degrees overnight or at 18 degrees for 24 h after initial growth for 6-8 h at 37 degrees. Cells were harvested by centrifugation at 4000×g and lysed by sonication after resuspension of the cells in lysis buffer (100 mM Tris pH 8.0, 200 mM NaCl, 50 mM Imidazole pH 8.0) containing protease inhibitors (Thermo Scientific) and Bovine pancreas DNaseI (Sigma-Aldrich). Proteins were purified by Immobilized Metal Affinity Chromatography. Cleared lysates were incubated with 2-4 ml nickel NTA beads (Qiagen) for 20-40 minutes before washing beads with 5-10 column volumes of lysis buffer, 5-10 column volumes of high salt buffer (10 mM Tris pH 8.0, 1 M NaCl) and 5-10 column volumes of lysis buffer. Proteins were eluted with 10 ml of elution buffer (20 mM Tris pH 8.0, 100 mM NaCl, 500 mM Imidazole pH 8.0).

Designs were finally polished using size exclusion chromatography (SEC) on either Superdex™ 200 Increase 10/300GL or Superdex™ 75 Increase 10/300GL columns (GE Healthcare) using 20 mM Tris pH 8.0, 100 mM NaCl or 20 mM Tris pH 8.0, 300 mM NaCl. Cyclic assemblies of C3 and C4 symmetries were purified using a Superose™ 6 increase 10/300GL (GE Healthcare). The two component C4 rings were SEC purified in 25 mM Tris pH 8.0, 300 mM NaCl. Peak fractions were verified by SDS-PAGE and LC/MS and stored at concentrations between 0.5-10 mg/ml at 4 degrees or flash frozen in liquid nitrogen for storage at −80. Designs that precipitated at low concentration upon storage at 4 degrees could in general be salvaged by increasing the salt concentration to 300-500 mM NaCl.

For structural studies, designs with a polyhistidine tag and TEV recognition site were cleaved using TEV protease (his6-TEV). TEV cleavage was performed in a buffer containing 20 mM Tris pH 8.0, 100 mM NaCl and 1 mM TCEP using 1% (w/w) his6-TEV and allowed to proceed o/n at room temperature. Uncleaved protein and his6-TEV were separated from cleaved protein using IMAC followed by SEC. Designs carrying a C-terminal SNAC-polyhistine tag (GGSHHWGS( . . . )HHHHHH) (SEQ ID NOs: 193, 194) were cleaved chemically via on-bead nickel assisted cleavage; nickel bound designs were washed with 10 CV of lysis buffer followed by 5 CV of 20 mM Tris pH 8.0, 100 mM NaCl. Proteins were subsequently washed with 5 CV of SNAC buffer (100 mM CHES, 100 mM Acetone oxime, 100 mM NaCl, pH 8.6). Beads were next incubated with 5 CV SNAC buffer+2 mM NiCl₂ for more than 12 hours at room temperature on a shaking platform to allow cleavage to take place. Next, the flow through containing cleaved protein was collected. The flow throughs of two additional washes (SNAC buffer/SNACbuffer+50 mM Imidazole) of 3-5 CV were also collected to harvest any remaining weakly bound protein. Cleaved proteins were finally purified by SEC.

### Luciferase Binding Assays

Assays were performed in 20 mM sodium phosphate, 100 mM NaCl, pH 7.4, 0.05% v/v Tween 20. Reactions were assembled in 96 well plates (Corning, cat #3686) in the presence of Nano-Glo™ substrate (Promega, cat. #N1130) diluted 100× or 500× for kinetics and endpoint measurements respectively (see supplement for detailed information). Luminescence was recorded on a Synergy Neo2 plate reader (BioTek). Kinetic assays were performed under pseudo first-order conditions, with the final concentration of one protein at 1 nM and the other at 10 nM. Dead times between substrate addition and data acquisition were typically 15-30 s. For long kinetic measurements (FIG. 11A), mastermixes of the protein complexes were made and aliquots were sampled at regular intervals. Data were fitted to a single exponential decay function:

$$S = A * exp ( - kobs * t ) + B$$

where t is time, S is the luminescence signal, and the fitted parameters are: A the amplitude, k_{obs} the observed rate constant, and B the endpoint luminescence.

Equilibrium binding reactions were incubated overnight at room temperature before adding substrate and immediately measuring luminescence. The data was fitted to the following equation to obtain K_{d} values:

$$S = S 0 + S 1 * fAB + a 2 * BT * S 2$$

$$fAB = ( AT + BT + Kd - {( AT + BT + Kd )}^{2} - 4 ATBT ) / ( 2 AT )$$

where A_{T} and B_{T} are the total concentrations of each species (A_{T}=1 nM, B_{T} is the titrated species), and S is the observed signal. The fitted parameters are: S₀ the pre-saturation baseline, S₁ the post-saturation baseline, a₂ and S₂ the correction terms, and K_{d} the equilibrium dissociation constant.

ABC complex equilibrium binding experiments were performed using the concentration indicated in the figure legend of FIG. 11G for the constant components, and titrating B. Reactions were incubated overnight before adding substrate and data acquisition (for details on the modeling of ABC kinetics see supplement). For the ABC reconfiguration kinetics (FIG. 5B) components A and C were briefly pre-incubated in the presence of substrate, before adding component B to start the reaction. At equilibrium component B′ was added to the reactions, and data acquisition was resumed until dissociation was complete.

### Enzymatic Protein Biotinylation

Avi-tagged (GLNDIFEAQKIEWHE (SEQ ID NO: 194), see supplement) proteins were purified as described above. The BirA500 (Avidity, LLC) biotinylation kit was used to biotinylate 840 uL of protein from the IMAC elution in a 1200 uL (final volume) reaction according to the manufacturer's protocol. Reactions were incubated at 4 degrees C. o/n and purified using size exclusion chromatography on a Superdex™ 200 10/300 Increase GL (GE Healthcare) or S7510/300 Increase GL (GE Healthcare) in SEC buffer (20 mM Tris pH 8.0, 100 mM NaCl).

### Biolayer Interferometry

Biolayer interferometry experiments were performed on an OctetRED96 BLI system (ForteBio, Menlo Park, CA). Streptavidin coated biosensors were first equilibrated for at least 10 minutes in Octet buffer (10 mM HEPES pH 7.4, 150 mM NaCl, 3 mM EDTA, 0.05% Surfactant P20) supplemented with 1 mg/ml Bovine Serum Albumin (SigmaAldrich). Enzymatically biotinylated designs were immobilized onto the biosensors by dipping the biosensors into a solution with 10-50 nM protein for 30-120 s. This was followed by dipping in fresh octet buffer to establish a baseline for 120 s. Titration experiments were performed at 25° C. while rotating at 1,000 r.p.m. Association of designs was allowed by dipping biosensors in solutions containing designed protein diluted in octet buffer until equilibrium was approached followed by dissociation by dipping the biosensors into fresh buffer solution in order to monitor the dissociation kinetics. Steady-state and global kinetic fits were performed using the manufacturer's software (Data Analysis 9.1) assuming a 1:1 binding model.

### SEC Binding Assays

Complexes and individual components were diluted in 20 mM Tris pH 8.0, 100 mM NaCl. After o/n equilibration of the mixtures at room temperature or 4 degrees C., 500 ul of sample was injected onto a Superdex™ 200 10/300 increase GL (dimers, linear assemblies) or Superose™ 6 increase 10/300 GL (symmetric assemblies) (all columns from GE healthcare) using the absorbance at 230 nm or 473 nm (for GFP tagged components) as read-out. Dimers were mixed at monomer concentrations of 5 μM or higher. Trimer and ABCD tetramer mixtures contained 5 μM of the bivalent connector, and 7.5 μM of each terminal cap (lower absolute concentrations with the same ratios were used for some trimers). ABCA tetramer mixtures contained 5 μM per bivalent connector and 15 μM terminal cap. The hexamer mixture contained 3 μM of components C and D, 3.6 μM of B and E, and 4.4 μM of A and F. The branched assembly shown in FIG. 4A contained 2.8 μM of the trivalent connector and 4 μM of each cap. For the exchange experiment shown in FIG. 5A, the ABC trimer was preincubated at concentrations of 6 μM B and 9 μM each of A and C. C′ was then added to reach a final concentration of 2 μM B, 3 μM each of A and C, and 6 μM C′.

### Native Mass Spectrometry

Sample purity, integrity, and oligomeric state was analyzed by on-line buffer exchange MS in 200 mM ammonium acetate using a Vanquish ultra-high performance liquid chromatography system coupled to a Q Exactive™ ultra-high mass range Orbitrap™ mass spectrometer (Thermo Fisher Scientific). A self-packed buffer exchange column was used (P6 polyacrylamide gel, BioRad). The recorded mass spectra were deconvolved with UniDec™ version 4.2+.

### Crystal Structure Determination

For all structures, starting phases were obtained by molecular replacement using Phaser™. Diffraction images were integrated using XDS (47) or HKL2000 (48) and merged/scaled using Aimless (49). Structures were refined in Phenix™ (50) using phenix.autobuild and phenix.refine or Refmac (51). Model building was performed using COOT (52).

Proteins were crystallized using the vapor diffusion method at room temperature. LHD29 crystals grew in 0.2M Sodium Iodide, 20% PEG3350, LHD29A53/B53 crystals in E5 and LHD101A53/B4 crystals in 2.4M Sodium Malonate pH 7.0. Crystals were harvested and cryoprotected using 20% PEG200 for LHD29, 20% PEG400 for LHD29A53/B53 and 20% glycerol for LHD101A53/B4 before data was collected at the Advanced Light Source (Berkeley, USA). The structures were solved by molecular replacement using either computationally designed models of individual chains A or B or the full heterodimer complex as search models.

### Electron Microscopy

SEC peak fractions were concentrated prior to negative stain EM screening. Samples were then immediately diluted 5 to 150 times in TBS buffer (25 mM Tris pH 8.0, 25 mM NaCl) depending on sample concentration. A final volume of 5 μL was applied to negatively glow discharged, carbon-coated 400-mesh copper grids (01844-F, TedPella, Inc.), then washed with Milli-Q™ Water and stained using 0.75% uranyl formate as previously described (53). Air-dried grids were imaged on a FEI Talos L120C TEM (FEI Thermo Scientific, Hillsboro, OR) equipped with a 4K×4K Gatan OneView™ camera at a magnification of 57,000× and pixel size of 2.51. Micrographs were imported into CisTEM software or cryoSPARC™ software and a circular blob picker was used to select particles which were then subjected to 2D classification. Ab initio reconstruction and homogeneous refinement in Cn symmetry were used to generate 3D electron density maps (54, 55).

### Additional Methods for the Luciferase Assay

### Constructs

Split luciferase reporter constructs were ordered as synthetic genes from Genscript. Each design was N-terminally fused to a sfGFP (for protein quantification in lysate), and C-terminally fused to either smBiT or lgBiT of the split luciferase components. A Strep-tag was included at the N-terminus for purification, and a GS-linker was inserted between the design and the split luciferase component.

### Expression for Multiplexed Assay

Plasmids were transformed into Lemo21(DE3) cells (New England Biolabs), and grown in 96 deepwell plates overnight at 37° C. in 1 mL of LB containing 50 ug/mL of kanamycin sulfate. The next day, 100 uL of overnight cultures were used to inoculate 96 deepwell plates containing 900 uL of TBII medium (MP Biomedicals) with 50 ug/mL of kanamycin sulfate, and the cultures were grown for 2 h at 37° C. before induction with 0.1 mM IPTG. Protein expression was carried out at 37° C. for 4 h before the cells were harvested by centrifugation (4,000×g, 5 min). Cell pellets were resuspended in 100 uL of lysis buffer (10 mM sodium phosphate, 150 mM NaCl, pH 7.4, 1 mg/mL lysozyme, 0.1 mg/mL DNAse I, 5 mM MgCl₂, 1 tablet/50 mL of complete protease inhibitor (Roche), 0.05% v/v Tween 20), and cell were lysed by performing three freeze/thaw cycles (1 h incubations at 37° C. followed by freezing at −80° C.). The lysate was cleared by centrifugation (4,000×g, 20 min), and the soluble fraction transferred to a 96 well assay plate (Corning, cat #3991). Concentrations of the constructs in soluble lysate were determined by sfGFP fluorescence using a calibration curve.

### Lysate Production for Multiplexed Assay

Neutral lysate for preparing serial dilutions was prepared by transforming Lemo21(DE3) with the pUC19 plasmid. Transformations were used to inoculate small overnight cultures, which were used to inoculate 0.5 L TBII cultures (all cultures contained 50 ug/mL of carbenicillin). Cells were grown for 24 h at 37° C. before being harvested. Pellets were resuspended in the same lysis buffer, followed by sonication. The lysate density was adjusted with lysis buffer to have its OD280 matching pUC19 control wells from the 96 well expression plate.

### Expression and Purification

Plasmids were transformed into Lemo21 (DE3) cells, and used directly to inoculate 50 mL of auto-induction media (TBII supplemented with 0.5% w/v glucose, 0.05% w/v glycerol, 0.2% w/v lactose monohydrate, and 2 mM MgSO₄. 50 ug/mL kanamycin sulfate). The cultures were incubated at 37° C. for 20-24 h, before harvesting the cells by centrifugation (4,000×g, 5 min). Cells were resuspended in 10 mL of lysis buffer (100 mM Tris, 150 mM NaCl, pH 8, 0.1 mg/mL lysozyme, 0.01 mg/mL DNAse I, 1 mM PMSF) and lysed by sonication. The insoluble fraction was cleared by centrifugation (16,000×g for 45 min), and the proteins were purified from the soluble fraction by affinity chromatography using Strep-Tactin XT Superflow™ High-Capacity resin (IBA Lifesciences). Elutions were performed with 100 mM Tris, 150 mM NaCl, 50 mM biotin, pH 8, and the proteins were further purified by size-exclusion chromatography using a Superdex™ 200 10/300 increase column equilibrated with 20 mM sodium phosphate, 100 mM NaCl, pH 7.4, 0.05% v/v Tween 20.

### Binding Assays

All assays were performed in 20 mM sodium phosphate, 100 mM NaCl, pH 7.4, 0.05% v/v Tween 20. Depending on the source of the protein used in the assay (purified components or lysate), soluble lysate components were also present. Reactions were assembled in 96 well plates (Corning, cat #3686) in the presence of Nano-Glo™ substrate (Promega, cat. #N1130) diluted 100× or 500× for kinetics and endpoint measurements respectively, and the luminescence signal was recorded on a Synergy Neo2 plate reader (BioTek).

Kinetic binding assays were performed under pseudo first-order conditions, with the final concentration of one protein at 1 nM and the other at 10 nM. Stock solutions were mixed in a 1:1 volume ratio in the presence of substrate, and the dead-time between mixing and starting the measurement (typically 15-30 s) added during data-processing. For long kinetic measurements (FIG. 11A), the proteins were pre-mixed, and kept in a sealed tube at room temperature over the course of the experiment. Aliquots were taken at regular intervals, mixed with substrate, and immediately recorded. All kinetic measurements were fitted to a single exponential decay function:

$$S = A * exp ( - kobs * t ) + B$$

where t is time (the independent variable), S is the observed luminescence signal (the dependent variable), and the fitted parameters are: A the amplitude, k_{obs} the observed rate constant, and B the endpoint luminescence.

Equilibrium binding assays were performed with one component kept constant at 1 nM while titrating the other protein. Serial dilutions curves were prepared over 12 points, with a ¼ dilution factor between each step. The concentration of protein in the soluble lysate provided the highest concentration point of the curve. To avoid serial dilution of the other lysate components, all stocks were prepared with neutral lysate. The assembled plates were incubated overnight at room temperature before adding substrate and immediately measuring luminescence. The data was fitted to the following equation to obtain K_{d} values:

$$S = S 0 + S 1 * fAB + a 2 * BT * S 2$$

$$fAB = ( AT + BT + Kd - {( AT + BT + Kd )}^{2} - 4 ATBT ) / ( 2 AT )$$

where A_{T} and B_{T} are the total concentrations of each species (the independent variables, A_{T}=1 nM, B_{T} is the titrated species), and S is the observed signal (the dependent variable). The fitted parameters are: S₀ the pre-saturation baseline, S₁ the post-saturation baseline, a₂ and S₂ the correction terms, and K_{d} the equilibrium dissociation constant.

Specificity matrices were obtained by preparing all combinations of smBiT and lgBiT proteins at 100 nM and 1 nM final concentrations respectively. The reactions were incubated overnight at room temperature before adding substrate and immediately measuring luminescence.

Ternary complex equilibrium binding experiments were performed with pure protein, using the concentration indicated in the figure legend of FIG. 11G for the constant components, and titratring B. After assembly, the plates were incubated overnight before adding substrate and immediately measuring luminescence.

Ternary complex reconfiguration kinetics (FIG. 5B) were measured with pure proteins. Components A and C were briefly pre-incubated in the presence of substrate, before adding component B to start the reaction. Once the association was complete, the assay plate was briefly taken out of the plate reader, component B′ was added to the reactions, and data acquisition was resumed until dissociation was complete.

### Simulation of Ternary Complex

Systems of ordinary differential equations describing the kinetics of interactions between the species involved in the formation of the ternary complex were numerically integrated using integrate.odeint( ) as implemented in Scipy (version 1.6.3). Steady-state values were used to determine the distribution of species at thermodynamic equilibrium.

The ternary system is composed of the following species: A, B, C, AB, BC, ABC. The following set of equations was used to describe the system:

$$d [ A ] dt = - k 1 [ A ] [ B ] + k - 1 [ AB ] - k 1 [ A ] [ BC ] + k - 1 [ ABC ]$$

$$d [ B ] dt = - k 1 [ A ] [ B ] + k - 1 [ AB ] - k 2 [ B ] [ C ] + k - 2 [ BC ]$$

$$d [ C ] dt = - k 2 [ B ] [ C ] + k - 2 [ BC ] - k 2 [ AB ] [ C ] + k - 2 [ ABC ]$$

$$d [ AB ] dt = k 1 [ A ] [ B ] - k - 1 [ AB ] + k - 2 [ ABC ] - k 2 [ AB ] [ C ]$$

$$d [ BC ] dt = k 2 [ B ] [ C ] - k - 2 [ BC ] + k - 1 [ ABC ] - k 1 [ A ] [ BC ]$$

$$d [ ABC ] dt = k 1 [ A ] [ BC ] - k 1 [ ABC ] + k 2 [ AB ] [ C ] - k - 2 [ ABC ]$$

where ki describe bimolecular association rate constants and k-irepresent unimolecular dissociation rate constants. K1=k−1/k1, and K2=k−2/k2 describe the affinity of the A:B and B:C interfaces respectively.

1. A polypeptide comprising an amino acid sequence at least 25% identical to the amino acid sequence selected from the group consisting of SEQ ID NOS:1-28, not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity.

2. The polypeptide of claim 1, wherein 1, 2, 3, 4, 5, 6, 7, 8, 9, 10, 11, 12, 13, 14, 15, 16, 17, 18, 19, 20, 21, or all of identified interface amino acid residues are identical at that residue position to the reference polypeptide.

3. The polypeptide of claim 1, wherein 1, 2, 3, 4, or 5 N-terminal and/or C-terminal amino acid residues are not included when determining the percent identity relative to the reference polypeptide; or wherein all residues are included when determining the percent identity relative to the reference polypeptide.

4. (canceled)

5. The polypeptide of claim 1, wherein amino acid substitutions relative to the reference polypeptide are conservative substitutions.

6. A heterodimer-forming polypeptide, comprising the amino acid sequence selected from the group consisting of SEQ ID NOS: SEQ ID NOS:29-33, 35-55, and 190-191 or comprising the amino acid sequence of any one of SEQ ID NOS:29, 35-55, and 190-191, wherein any functional domains added fused to the polypeptides (whether N-terminal, C-terminal, or internal other than within the interface region), and wherein the 1, 2, 3, 4, or 5 N-terminal and/or C-terminal amino acid residues may be present or absent.

7. A heterodimer-forming polypeptide, comprising the amino acid sequence selected from the group consisting of SEQ ID NOS:56-77, or comprising the amino acid sequence of any one of SEQ ID NOS:56 and 60-77.

8. A fusion protein, comprising:
(a) the polypeptide claim 1; and
(b) a second polypeptide; optionally including an amino acid linker between the polypeptide and the second polypeptide.

9. The fusion protein of claim 8, wherein the second polypeptide comprises a repeat polypeptide.

10. The fusion protein of claim 9 wherein the repeat protein comprises an amino acid sequence at least 50% identical to the amino acid sequence selected from the group consisting of SEQ ID NOS:78-89.

11. The fusion protein of claim 8, further comprising a third functional polypeptide C-terminal to the repeat protein, or N-terminal to the polypeptide, comprising an amino acid sequence at least 25% identical to the amino acid sequence selected from the group consisting of SEQ ID NOS:1-28.

12. The fusion protein of claim 8, comprising an amino acid sequence at least 25% identical to the amino acid sequence selected from the group consisting of SEQ ID NOS: 90-189 and 196-199, not including any functional domains added (whether N-terminal, C-terminal, or internal other than within the interface region), and wherein the 1, 2, 3, 4, or 5 N-terminal and/or C-terminal amino acid residues, and any N-terminal methionine residue, may be present or absent when considering the percent identity.

13. The fusion protein of claim 8, comprising an amino acid sequence at least 25% identical to the amino acid sequence selected from the group consisting of SEQ ID NOS: 90-189, not including any functional domains added (whether N-terminal, C-terminal, or internal other than within the interface region), and wherein the 1, 2, 3, 4, or 5 N-terminal and/or C-terminal amino acid residues, and any N-terminal methionine residue, may be present or absent when considering the percent identity

14. A nucleic acid encoding the polypeptide of claim 1.

15. An expression vector comprising the nucleic acid of claim 14 operatively linked to a suitable control sequence.

16. A host cell comprising the expression vector of claim 15.

17. A heterodimer, comprising two polypeptides according to claim 1, wherein the two polypeptides are capable of self-assembly to form a heterodimer.

18. The heterodimer of claim 17, wherein the two polypeptides or fusion proteins are a Chain A and Chain B pair comprising an amino acid sequence at least 25% identical to the amino acid sequence of selected from the following pairs (Chain A listed first; Chain B listed second), not including any functional domains added fused to the polypeptides (whether N-terminal, C-terminal, or internal other than within the interface region), and wherein the 1, 2, 3, 4, or 5 N-terminal and/or C-terminal amino acid residues may be present or absent when considering the percent identity:
(a) one of SEQ ID NOS:1-5 and SEQ ID NO:6;
(b) SEQ ID NO:7 and SEQ ID NO: 8;
(c) SEQ ID NO:9 and SEQ ID NO: 10;
(d) SEQ ID NO:11 and SEQ ID NO: 12;
(e) SEQ ID NO:13 and SEQ ID NO: 14;
(f) SEQ ID NO:15 and SEQ ID NO: 16;
(g) SEQ ID NO:17 and SEQ ID NO: 18;
(h) SEQ ID NO:19 and SEQ ID NO: 20;
(i) SEQ ID NO:21 and SEQ ID NO:22;
(j) SEQ ID NO:23 and SEQ ID NO:24;
(k) SEQ ID NO:25 and SEQ ID NO:26; and
(l) SEQ ID NO:27 and SEQ ID NO:28.

19. The heterodimer of claim 17, wherein the two polypeptides or fusion proteins are a Chain A and Chain B pair comprising the amino acid sequence selected from the following pairs (Chain A listed first; Chain B listed second):
(a) one of SEQ ID NOS:29-32 and SEQ ID NO:33;
(b) SEQ ID NO:190 and SEQ ID NO:191;
(c) SEQ ID NO:35 and SEQ ID NO:36;
(d) SEQ ID NO:37 and SEQ ID NO:38;
(e) SEQ ID NO:39 and SEQ ID NO:40;
(f) SEQ ID NO:41 and SEQ ID NO: 42;
(g) SEQ ID NO:43 and SEQ ID NO:44;
(h) SEQ ID NO:46 and SEQ ID NO:47;
(i) SEQ ID NO:48 and SEQ ID NO:49;
(j) SEQ ID NO:50 and SEQ ID NO:51;
(k) SEQ ID NO:52 and SEQ ID NO: 53;
(l) SEQ ID NO:54 and SEQ ID NO:55;
(m) one of SEQ ID NO:56-59 and SEQ ID NO:60;
(n) SEQ ID NO:61 and SEQ ID NO:191;
(o) SEQ ID NO:62 and SEQ ID NO:63;
(p) SEQ ID NO:64 and SEQ ID NO:65;
(q) SEQ ID NO:66 and SEQ ID NO: 67;
(r) SEQ ID NO:68 and SEQ ID NO:69;
(s) SEQ ID NO:70 and SEQ ID NO: 71;
(t) SEQ ID NO:72 and SEQ ID NO: 73;
(u) SEQ ID NO:74 and SEQ ID NO:75; and
(v) SEQ ID NO:76 and SEQ ID NO:77.

20. An asymmetric hetero-oligomeric assembly comprising a plurality (2, 3, 4, 5, 6, 7, 8, 9, 10, or more) of the heterodimers of claim 17.

21. (canceled)

22. A method for making a heterodimer, comprising mixing two or more of the polypeptides of claim 1, resulting in self-assembly of the heterodimer.

23. (canceled)


SEQUENCE LISTING (199 sequences)

SEQ ID NO: 1 - protein, 75 aa, synthetic construct
  REGION 1..75: note = Synthetic
GRQEKVLKSIEETVRKMGVTMETHRSGNEVKVVIKGLHIKQQRQLYRDVRETSKKQGVETEIEVEGDTVTIVVRE

SEQ ID NO: 2 - protein, 75 aa, synthetic construct
  REGION 1..75: note = Synthetic
GRQEKVLKSIEETVRKMGVTMETHRSGNEVKVVIKGLHIKQMRQLYRDVRETSKKQGVETEIEVEGDTVTIVVRE

SEQ ID NO: 3 - protein, 75 aa, synthetic construct
  REGION 1..75: note = Synthetic
GRQEKVLKSIEETVRKMGVTMETHRSGNEVKVVIKGLHIKQQVQLYRDVRETSKKQGVETEIEVEGDTVTIVVRE

SEQ ID NO: 4 - protein, 75 aa, synthetic construct
  REGION 1..75: note = Synthetic
GRQEKVLKSIEETVRKMGVTMETHRSGNEVKVVIKGLHIKQQRQLYRDVRETSKKQGVETEIEVEGDTQTIVVRE

SEQ ID NO: 5 - protein, 75 aa, synthetic construct
  REGION 1..75: note = Synthetic
GRQEKVLKSIEETVRKMGVTMETHRSGNEVKVVIKGLHIKQQRQLYRDVRETSKKQGVETEIEVEGDTVWIVVRE

SEQ ID NO: 6 - protein, 75 aa, synthetic construct
  REGION 1..75: note = Synthetic
GRQEKVLKSIEETVRKMGVTMETHRSGNEVKVVIKGLHESQQEQLLEDVLRTAEKQGVRVRIRFKGDTVTIVVRE

SEQ ID NO: 7 - protein, 75 aa, synthetic construct
  REGION 1..75: note = Synthetic
GRQEKVLKSIEETVRKMGVTMETHRSGNKVKVVIKGLHESQQEQLRKDVHETLRKQGVVAVTQKHGDTVTIYVTE

SEQ ID NO: 8 - protein, 65 aa, synthetic construct
  REGION 1..65: note = Synthetic
SVEFHIVNISEEQRQRIEEYVRRISKKEGTEVRFEKRDGELTIEVKNLHEKRLQEILEYIERVNK

SEQ ID NO: 9 - protein, 80 aa, synthetic construct
  REGION 1..80: note = Synthetic
TDELLERLRQLFEELHERGTEIVVEVHINGRKTEIEVQGIDKRLLKIILEVIREEIEREGSSEVEVNVHSGGQTWTFNEK

SEQ ID NO: 10 - protein, 75 aa, synthetic construct
  REGION 1..75: note = Synthetic
GRQEKVLKSIEETVRKMGVTMETHRSGNEVKVVIKGLHKSQQEQLLKDVLKTANKQGVNVHISFRGDTVTIRVRE

SEQ ID NO: 11 - protein, 65 aa, synthetic construct
  REGION 1..65: note = Synthetic
TTNFHLINGSEEARQRIEEYVRRISKKEGTEVHFEKSDGTLEIRVKNLHEKREREIKEYIERVLL

SEQ ID NO: 12 - protein, 65 aa, synthetic construct
  REGION 1..65: note = Synthetic
NTHFIVVHGSEEARQRAEEYVRRISKKEGTEVRFEKKDGLLSIEVKNLSEERQREIQEYLQRVQK

SEQ ID NO: 13 - protein, 124 aa, synthetic construct
  REGION 1..124: note = Synthetic
GRQEKVLKSIEETVRKMGVEMLTFRAGNAVIVVIRGLHPEQAKQLLRDVSQTAHKQGVTVTLTFHGDVVFILVLVGASEEEQKHMQERIQELARIIHEAKRRGVSEEQLREIAEKMAKEIQEWG

SEQ ID NO: 14 - protein, 243 aa, synthetic construct
  REGION 1..243: note = Synthetic
DVEWRYTNISEETQQKSAEFVLEIALRAGTGVTFTTRQGELQIQVHNLDELLAIAMLCYTLGLLLGDHRVQELAKRAVEAWERGDEERVKKLLIEALKRLVETAEEVVRERPGSNLAKLALEIILRAAEALARAEDPESLKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 15 - protein, 124 aa, synthetic construct
  REGION 1..124: note = Synthetic
GRQEKVLKSIEETVRKMGVRMLTHRGGNAVIVVIEGLHPSQIKQLMQDVIKTAKKQGVTVTITVSGDIVVIMVVVGASDEEQEEARRLVQEIARALQEAKRKGANEEQLEQLLRELLERAEREG

SEQ ID NO: 16 - protein, 220 aa, synthetic construct
  REGION 1..220: note = Synthetic
TVTFDITNIDWKSAELIMLAVYDIAQQEGTDVTFSFKEGELQITVKNLHEKWKRLIEMLIEACRRAQDPDPESLKEAVRIAEELVRLHPGNPLARAALKVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 17 - protein, 80 aa, synthetic construct
  REGION 1..80: note = Synthetic
TDELLERLRQLFEELHERGTIIIVEVHINGERQTKYLILAPKEELKKHLERIREKIEREGSSEVEVKVTSGGTTWTFNEK

SEQ ID NO: 18 - protein, 246 aa, synthetic construct
  REGION 1..246: note = Synthetic
PHQFYVYQIDEHVAQLIEKFVRDISRREGTEVRFEKRDGQLEIEVKNLHEAQAIAIGIYIMILLLHQSGTSEDEIAEEIAKLIKGFIEHLKREGSSYEVICEAVAAAVAAIVKALKGCGTSEDEIAEIVARVISEVIRTLKESGSSYEVICECVARIVAEIVQALKESGTSEDEIAEIVARVISEVIRTLKESGSSYEVIKECVQRIVEEIVEALKRSGTSEDEINEIVRRVKSEVERTLKESGSS

SEQ ID NO: 19 - protein, 120 aa, synthetic construct
  REGION 1..120: note = Synthetic
GRQEKVLKSIEETVRKMGVTMLTHRHGNVVFVVILGLHKQQALQLLRDVHRTAHKQGVTLSITFSGDIVVIAVTVGASEEEKKEVRKIVKEIAKQLRHAETEEEAKEIVQRVIEEWQEEG

SEQ ID NO: 20 - protein, 220 aa, synthetic construct
  REGION 1..220: note = Synthetic
TVTFDITNISHEAIEIILYGVLGIAAMEGTEVTFHSERGQLQIEVKNLHEKQKRNIEKLIEAALRAQSPDPEDLKEAVRIAEELVRAHPGTPLAHAALQVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 21 - protein, 65 aa, synthetic construct
  REGION 1..65: note = Synthetic
TWQWVLINISEEARQRIEEYVRRISKKEGTEVHFEKDDGVLHIRVKNLHEKRAREIHEYAKRVIL

SEQ ID NO: 22 - protein, 65 aa, synthetic construct
  REGION 1..65: note = Synthetic
SSIFLLSNVSEEARQRAEEYVRRISKKEGTEVRFEKDDGFLTIEVKNLSEERLREIAEYLWRVAV

SEQ ID NO: 23 - protein, 75 aa, synthetic construct
  REGION 1..75: note = Synthetic
GRQEKVLKSIEETVRKMGVTMETHRSGNTVKVVIKGLHESQQEQLHKDVEETVQKEGVFVLVSHHGDTVTIQVYE

SEQ ID NO: 24 - protein, 185 aa, synthetic construct
  REGION 1..185: note = Synthetic
SHSFILGQASEEARQEIEEVVEAISRKLGTEVRFEKKDGTLHIEVKNLHDEYAQLIADAILLIILAQESDDSEAKKVARLALEIVAQLPNTELAHEALKLAEEALKSTDSEALKVVYLALRIVQQLPDTELAREALELAKEAVKSTDQEALKSVYEALQRVQDKPNTEEARESLERAKEDVKSTD

SEQ ID NO: 25 - protein, 124 aa, synthetic construct
  REGION 1..124: note = Synthetic
GRQEKVLKSIEETVRKMGVRMLTHRGGNAVIVVIEGLHPSQAEQLLRDVHRTAKKQGVTVHLVFTGDIVVIMVVVGASEEEQEEMHRLVREIAEALHEAKRKGANEEQLEQLLRELLERAEREG

SEQ ID NO: 26 - protein, 237 aa, synthetic construct
  REGION 1..237: note = Synthetic
DVEWRFTNVSEEEQEKLARFVLQVAQLAGTQVIFTTRPGELRIRVHNLDELLALAIELYAQGLRLGDKHVQHLAKKAIEAILRGDRKLARFLLEAARAMSRATERPGSNLAKKALEEILRLAEELAKDPDPESLKAAVHCAEFVVRYQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 27 - protein, 100 aa, synthetic construct
  REGION 1..100: note = Synthetic
TKEELKRAIEEAHRKGDKEKLKEVIKRAQEEGDEEVYREAIQALAKLIAEEAGVDDVRVEVHNGRVRLEIRGQSQAVVRVATEVVTELGKLGIRVTVQLG

SEQ ID NO: 28 - protein, 220 aa, synthetic construct
  REGION 1..220: note = Synthetic
TVTFDITNIDDKSTKLIATAVIHIAGREGTTVHFQGHDGQLEIEVKNLHEKWKRLIEMLIEACRRAQDPDPESLKEAVRIAEELVRLHPGNMLAEAALKVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 29 - protein, 75 aa, synthetic construct
  REGION 1..75: note = Synthetic
  REGION 1..38: note = MISC_FEATURE - X is any amino acid
  SITE 41: note = MISC_FEATURE - X is any amino acid
  SITE 45: note = MISC_FEATURE - X is any amino acid
  REGION 48..49: note = MISC_FEATURE - X is any amino acid
  REGION 51..53: note = MISC_FEATURE - X is any amino acid
  REGION 55..58: note = MISC_FEATURE - X is any amino acid
  SITE 60: note = MISC_FEATURE - X is any amino acid
  REGION 68..75: note = MISC_FEATURE - X is any amino acid
XXXXXXXXXXXXXXXXXXXXXXXXXXXXXXXXXXXXXXIKXQRQXYRXXRXXXKXXXXEXEIEVEGDXXXXXXXX

SEQ ID NO: 30 - protein, 75 aa, synthetic construct
  REGION 1..75: note = Synthetic
  REGION 1..38: note = MISC_FEATURE - X is any amino acid
  SITE 41: note = MISC_FEATURE - X is any amino acid
  SITE 45: note = MISC_FEATURE - X is any amino acid
  REGION 48..49: note = MISC_FEATURE - X is any amino acid
  REGION 51..53: note = MISC_FEATURE - X is any amino acid
  REGION 55..58: note = MISC_FEATURE - X is any amino acid
  SITE 60: note = MISC_FEATURE - X is any amino acid
  REGION 68..75: note = MISC_FEATURE - X is any amino acid
XXXXXXXXXXXXXXXXXXXXXXXXXXXXXXXXXXXXXXIKXMRQXYRXXRXXXKXXXXEXEIEVEGDXXXXXXXX

SEQ ID NO: 31 - protein, 75 aa, synthetic construct
  REGION 1..75: note = Synthetic
  REGION 1..38: note = MISC_FEATURE - X is any amino acid
  SITE 41: note = MISC_FEATURE - X is any amino acid
  SITE 45: note = MISC_FEATURE - X is any amino acid
  REGION 48..49: note = MISC_FEATURE - X is any amino acid
  REGION 51..53: note = MISC_FEATURE - X is any amino acid
  REGION 55..58: note = MISC_FEATURE - X is any amino acid
  SITE 60: note = MISC_FEATURE - X is any amino acid
  REGION 68..75: note = MISC_FEATURE - X is any amino acid
XXXXXXXXXXXXXXXXXXXXXXXXXXXXXXXXXXXXXXIKXQVQXYRXXRXXXKXXXXEXEIEVEGDXXXXXXXX

SEQ ID NO: 32 - protein, 75 aa, synthetic construct
  REGION 1..75: note = Synthetic
  REGION 1..38: note = MISC_FEATURE - X is any amino acid
  SITE 41: note = MISC_FEATURE - X is any amino acid
  SITE 45: note = MISC_FEATURE - X is any amino acid
  REGION 48..49: note = MISC_FEATURE - X is any amino acid
  REGION 51..53: note = MISC_FEATURE - X is any amino acid
  REGION 55..58: note = MISC_FEATURE - X is any amino acid
  SITE 60: note = MISC_FEATURE - X is any amino acid
  REGION 68..75: note = MISC_FEATURE - X is any amino acid
XXXXXXXXXXXXXXXXXXXXXXXXXXXXXXXXXXXXXXIKXMVQXYRXXRXXXKXXXXEXEIEVEGDXXXXXXXX

SEQ ID NO: 33 - protein, 75 aa, synthetic construct
  REGION 1..75: note = Synthetic
  REGION 1..38: note = MISC_FEATURE - X is any amino acid
  REGION 40..41: note = MISC_FEATURE - X is any amino acid
  SITE 45: note = MISC_FEATURE - X is any amino acid
  SITE 48: note = MISC_FEATURE - X is any amino acid
  SITE 52: note = MISC_FEATURE - X is any amino acid
  REGION 55..58: note = MISC_FEATURE - X is any amino acid
  REGION 67..75: note = MISC_FEATURE - X is any amino acid
XXXXXXXXXXXXXXXXXXXXXXXXXXXXXXXXXXXXXXEXXQEQXLEXVLRXAEXXXXRVRIRFKGXXXXXVXXX

SEQ ID NO: 34 - skipped

SEQ ID NO: 35 - protein, 80 aa, synthetic construct
  REGION 1..80: note = Synthetic
  REGION 1..30: note = MISC_FEATURE - X is any amino acid
  SITE 42: note = MISC_FEATURE - X is any amino acid
  SITE 45: note = MISC_FEATURE - X is any amino acid
  SITE 49: note = MISC_FEATURE - X is any amino acid
  SITE 53: note = MISC_FEATURE - X is any amino acid
  REGION 56..80: note = MISC_FEATURE - X is any amino acid
XXXXXXXXXXXXXXXXXXXXXXVXXVXIXXRKTEIEVQGIDXRLXKIIXEVIXEEXXXXXXXXXXXXXXXXXXXXXXXXX

SEQ ID NO: 36 - protein, 75 aa, synthetic construct
  REGION 1..75: note = Synthetic
  REGION 1..38: note = MISC_FEATURE - X is any amino acid
  REGION 40..41: note = MISC_FEATURE - X is any amino acid
  REGION 44..45: note = MISC_FEATURE - X is any amino acid
  REGION 48..49: note = MISC_FEATURE - X is any amino acid
  SITE 52: note = MISC_FEATURE - X is any amino acid
  REGION 55..56: note = MISC_FEATURE - X is any amino acid
  REGION 67..73: note = MISC_FEATURE - X is any amino acid
  SITE 75: note = MISC_FEATURE - X is any amino acid
XXXXXXXXXXXXXXXXXXXXXXXXXXXXXXXXXXXXXXKXXQEXXLKXXLKXANXXGVNVHISFRGXXXXXRXRX

SEQ ID NO: 37 - protein, 65 aa, synthetic construct
  REGION 1..65: note = Synthetic
  REGION 11..36: note = MISC_FEATURE - X is any amino acid
  SITE 41: note = MISC_FEATURE - X is any amino acid
  REGION 45..55: note = MISC_FEATURE - X is any amino acid
  SITE 59: note = MISC_FEATURE - X is any amino acid
  REGION 62..63: note = MISC_FEATURE - X is any amino acid
TTNFHLINGSXXAXXXXXXXXXXXXXXXXXXXXXXXSDGTXEIRXXXXXXXXEXXIKEXIEXXLL

SEQ ID NO: 38 - protein, 65 aa, synthetic construct
  REGION 1..65: note = Synthetic
  REGION 10..36: note = MISC_FEATURE - X is any amino acid
  REGION 46..47: note = MISC_FEATURE - X is any amino acid
  SITE 49: note = MISC_FEATURE - X is any amino acid
  REGION 51..52: note = MISC_FEATURE - X is any amino acid
  SITE 55: note = MISC_FEATURE - X is any amino acid
  SITE 59: note = MISC_FEATURE - X is any amino acid
  SITE 62: note = MISC_FEATURE - X is any amino acid
NTHFIVVHGXXXAXXXAXXXXXXXXXXXXXXXXXXXKDGLLSIEVXXLXEXXQRXIQEXLQXVQK

SEQ ID NO: 39 - protein, 124 aa, synthetic construct
  REGION 1..124: note = Synthetic
  REGION 1..38: note = MISC_FEATURE - X is any amino acid
  SITE 41: note = MISC_FEATURE - X is any amino acid
  SITE 45: note = MISC_FEATURE - X is any amino acid
  SITE 48: note = MISC_FEATURE - X is any amino acid
  SITE 56: note = MISC_FEATURE - X is any amino acid
  REGION 67..69: note = MISC_FEATURE - X is any amino acid
  REGION 72..81: note = MISC_FEATURE - X is any amino acid
  REGION 83..85: note = MISC_FEATURE - X is any amino acid
  REGION 87..88: note = MISC_FEATURE - X is any amino acid
  REGION 91..92: note = MISC_FEATURE - X is any amino acid
  REGION 94..124: note = MISC_FEATURE - X is any amino acid
XXXXXXXXXXXXXXXXXXXXXXXXXXXXXXXXXXXXXXPEXAKQXLRXVSQTAHKXGVTVTLTFHGXXXFIXXXXXXXXXXQXXXQXXIQXXAXXXXXXXXXXXXXXXXXXXXXXXXXXXXXXX

SEQ ID NO: 40 - protein, 243 aa, synthetic construct
  REGION 1..243: note = Synthetic
  REGION 1..10: note = MISC_FEATURE - X is any amino acid
  REGION 12..13: note = MISC_FEATURE - X is any amino acid
  SITE 17: note = MISC_FEATURE - X is any amino acid
  SITE 20: note = MISC_FEATURE - X is any amino acid
  SITE 24: note = MISC_FEATURE - X is any amino acid
  SITE 28: note = MISC_FEATURE - X is any amino acid
  SITE 40: note = MISC_FEATURE - X is any amino acid
  SITE 43: note = MISC_FEATURE - X is any amino acid
  SITE 45: note = MISC_FEATURE - X is any amino acid
  REGION 47..243: note = MISC_FEATURE - X is any amino acid
XXXXXXXXXXEXXQQKXAEXVLEXALRXGTGVTFTTRQGXLQXQXHXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 41 - protein, 124 aa, synthetic construct
  REGION 1..124: note = Synthetic
  REGION 1..38: note = MISC_FEATURE - X is any amino acid
  REGION 40..41: note = MISC_FEATURE - X is any amino acid
  REGION 44..45: note = MISC_FEATURE - X is any amino acid
  REGION 48..49: note = MISC_FEATURE - X is any amino acid
  SITE 52: note = MISC_FEATURE - X is any amino acid
  REGION 55..56: note = MISC_FEATURE - X is any amino acid
  REGION 67..78: note = MISC_FEATURE - X is any amino acid
  REGION 80..81: note = MISC_FEATURE - X is any amino acid
  SITE 84: note = MISC_FEATURE - X is any amino acid
  REGION 87..88: note = MISC_FEATURE - X is any amino acid
  SITE 91: note = MISC_FEATURE - X is any amino acid
  REGION 94..96: note = MISC_FEATURE - X is any amino acid
  REGION 98..124: note = MISC_FEATURE - X is any amino acid
XXXXXXXXXXXXXXXXXXXXXXXXXXXXXXXXXXXXXXPXXIKXXMQXXIKXAKXXGVTVTITVSGXXXXXXXXXXXXDXXQEXARXXVQXIAXXXQXXXXXXXXXXXXXXXXXXXXXXXXXXX

SEQ ID NO: 42 - protein, 220 aa, synthetic construct
  REGION 1..220: note = Synthetic
  REGION 1..10: note = MISC_FEATURE - X is any amino acid
  SITE 13: note = MISC_FEATURE - X is any amino acid
  REGION 20..21: note = MISC_FEATURE - X is any amino acid
  SITE 30: note = MISC_FEATURE - X is any amino acid
  SITE 39: note = MISC_FEATURE - X is any amino acid
  SITE 41: note = MISC_FEATURE - X is any amino acid
  SITE 43: note = MISC_FEATURE - X is any amino acid
  SITE 45: note = MISC_FEATURE - X is any amino acid
  REGION 47..220: note = MISC_FEATURE - X is any amino acid
XXXXXXXXXXWKXAELIMLXXYDIAQQEGXDVTFSFKEXEXQXTXKXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 43 - protein, 80 aa, synthetic construct
  REGION 1..80: note = Synthetic
  REGION 1..19: note = MISC_FEATURE - X is any amino acid
  REGION 24..31: note = MISC_FEATURE - X is any amino acid
  REGION 42..43: note = MISC_FEATURE - X is any amino acid
  REGION 45..47: note = MISC_FEATURE - X is any amino acid
  REGION 49..68: note = MISC_FEATURE - X is any amino acid
  REGION 75..80: note = MISC_FEATURE - X is any amino acid
XXXXXXXXXXXXXXXXXXXTIIIXXXXXXXXRQTKYLILAPXXEXXXHXXXXXXXXXXXXXXXXXXKXTSGGTTXXXXXX

SEQ ID NO: 44 - protein, 246 aa, synthetic construct
  REGION 1..246: note = Synthetic
  REGION 10..39: note = MISC_FEATURE - X is any amino acid
  REGION 41..49: note = MISC_FEATURE - X is any amino acid
  REGION 51..52: note = MISC_FEATURE - X is any amino acid
  REGION 54..55: note = MISC_FEATURE - X is any amino acid
  REGION 62..63: note = MISC_FEATURE - X is any amino acid
  REGION 65..80: note = MISC_FEATURE - X is any amino acid
  REGION 90..91: note = MISC_FEATURE - X is any amino acid
  REGION 94..246: note = MISC_FEATURE - X is any amino acid
PHQFYVYQIXXXXXXXXXXXXXXXXXXXXXXXXXXXXXXQXXXXXXXXXEXXAXXIGIYIMXXLXXXXXXXXXXXXXXXXKLIKGFIEHXXREXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 45 - skipped

SEQ ID NO: 46 - protein, 120 aa, synthetic construct
  REGION 1..120: note = Synthetic
  REGION 1..38: note = MISC_FEATURE - X is any amino acid
  SITE 41: note = MISC_FEATURE - X is any amino acid
  REGION 44..45: note = MISC_FEATURE - X is any amino acid
  SITE 48: note = MISC_FEATURE - X is any amino acid
  SITE 52: note = MISC_FEATURE - X is any amino acid
  SITE 55: note = MISC_FEATURE - X is any amino acid
  SITE 56: note = MISC_FEATURE - X is any amino acid
  SITE 58: note = MISC_FEATURE - X is any amino acid
  REGION 67..69: note = MISC_FEATURE - X is any amino acid
  REGION 71..85: note = MISC_FEATURE - X is any amino acid
  SITE 87: note = MISC_FEATURE - X is any amino acid
  REGION 91..92: note = MISC_FEATURE - X is any amino acid
  REGION 94..96: note = MISC_FEATURE - X is any amino acid
  REGION 98..120: note = MISC_FEATURE - X is any amino acid
XXXXXXXXXXXXXXXXXXXXXXXXXXXXXXXXXXXXXXKQXALXXLRXVHRXAHXXGXTLSITFSGXXXVXXXXXXXXXXXKXXXRXIVKXXAXXXRXXXXXXXXXXXXXXXXXXXXXXX

SEQ ID NO: 47 - protein, 220 aa, synthetic construct
  REGION 1..220: note = Synthetic
  REGION 1..13: note = MISC_FEATURE - X is any amino acid
  REGION 16..17: note = MISC_FEATURE - X is any amino acid
  SITE 21: note = MISC_FEATURE - X is any amino acid
  SITE 24: note = MISC_FEATURE - X is any amino acid
  SITE 28: note = MISC_FEATURE - X is any amino acid
  SITE 30: note = MISC_FEATURE - X is any amino acid
  SITE 39: note = MISC_FEATURE - X is any amino acid
  REGION 40..41: note = MISC_FEATURE - X is any amino acid
  SITE 43: note = MISC_FEATURE - X is any amino acid
  SITE 45: note = MISC_FEATURE - X is any amino acid
  REGION 47..91: note = MISC_FEATURE - X is any amino acid
  REGION 93..220: note = MISC_FEATURE - X is any amino acid
XXXXXXXXXXHXXIEXXLYGXLGXAAMXGXEVTFHSERXXXQXEXKXXXXXXXXXXXXXXXXXXXXXXXXXXXXXXXXXXXXXXXXXXXXXPXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 48 - protein, 65 aa, synthetic construct
  REGION 1..65: note = Synthetic
  REGION 10..12: note = MISC_FEATURE - X is any amino acid
  REGION 14..36: note = MISC_FEATURE - X is any amino acid
  SITE 41: note = MISC_FEATURE - X is any amino acid
  REGION 45..52: note = MISC_FEATURE - X is any amino acid
  REGION 54..56: note = MISC_FEATURE - X is any amino acid
  REGION 58..59: note = MISC_FEATURE - X is any amino acid
  REGION 62..63: note = MISC_FEATURE - X is any amino acid
TWQWVLINIXXXAXXXXXXXXXXXXXXXXXXXXXXXDDGVXHIRXXXXXXXXAXXXHXXAKXXIL

SEQ ID NO: 49 - protein, 65 aa, synthetic construct
  REGION 1..65: note = Synthetic
  REGION 10..16: note = MISC_FEATURE - X is any amino acid
  REGION 18..36: note = MISC_FEATURE - X is any amino acid
  SITE 38: note = MISC_FEATURE - X is any amino acid
  SITE 44: note = MISC_FEATURE - X is any amino acid
  REGION 46..47: note = MISC_FEATURE - X is any amino acid
  SITE 49: note = MISC_FEATURE - X is any amino acid
  REGION 51..52: note = MISC_FEATURE - X is any amino acid
  SITE 55: note = MISC_FEATURE - X is any amino acid
  SITE 59: note = MISC_FEATURE - X is any amino acid
  SITE 62: note = MISC_FEATURE - X is any amino acid
SSIFLLSNVXXXXXXXAXXXXXXXXXXXXXXXXXXXDXGFLTIXVXXLXEXXLRXIAEXLWXVAV

SEQ ID NO: 50 - protein, 75 aa, synthetic construct
  REGION 1..75: note = Synthetic
  REGION 1..27: note = MISC_FEATURE - X is any amino acid
  REGION 30..48: note = MISC_FEATURE - X is any amino acid
  REGION 51..52: note = MISC_FEATURE - X is any amino acid
  REGION 66..69: note = MISC_FEATURE - X is any amino acid
  SITE 71: note = MISC_FEATURE - X is any amino acid
  SITE 73: note = MISC_FEATURE - X is any amino acid
XXXXXXXXXXXXXXXXXXXXXXXXXXXNTXXXXXXXXXXXXXXXXHXXVEXXVQKEGVFVLVSHHXXXXTXQXYE

SEQ ID NO: 51 - protein, 185 aa, synthetic construct
  REGION 1..185: note = Synthetic
  REGION 10..38: note = MISC_FEATURE - X is any amino acid
  SITE 41: note = MISC_FEATURE - X is any amino acid
  REGION 43..47: note = MISC_FEATURE - X is any amino acid
  SITE 49: note = MISC_FEATURE - X is any amino acid
  REGION 51..52: note = MISC_FEATURE - X is any amino acid
  SITE 55: note = MISC_FEATURE - X is any amino acid
  SITE 59: note = MISC_FEATURE - X is any amino acid
  SITE 62: note = MISC_FEATURE - X is any amino acid
  REGION 65..185: note = MISC_FEATURE - X is any amino acid
SHSFILGQAXXXXXXXXXXXXXXXXXXXXXXXXXXXXXGTXHXXXXXLXFXXAQXIADXILXIIXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 52 - protein, 124 aa, synthetic construct
  REGION 1..124: note = Synthetic
  REGION 1..38: note = MISC_FEATURE - X is any amino acid
  REGION 40..41: note = MISC_FEATURE - X is any amino acid
  REGION 44..45: note = MISC_FEATURE - X is any amino acid
  SITE 48: note = MISC_FEATURE - X is any amino acid
  REGION 68..85: note = MISC_FEATURE - X is any amino acid
  REGION 87..88: note = MISC_FEATURE - X is any amino acid
  SITE 91: note = MISC_FEATURE - X is any amino acid
  SITE 95: note = MISC_FEATURE - X is any amino acid
  REGION 98..124: note = MISC_FEATURE - X is any amino acid
XXXXXXXXXXXXXXXXXXXXXXXXXXXXXXXXXXXXXXPXXAEXXLRXVHRTAKKQGVTVHLVFTGDXXXXXXXXXXXXXXQXXXHXXVRXIAEXLHXXXXXXXXXXXXXXXXXXXXXXXXXXX

SEQ ID NO: 53 - protein, 237 aa, synthetic construct
  REGION 1..237: note = Synthetic
  REGION 1..10: note = MISC_FEATURE - X is any amino acid
  REGION 12..13: note = MISC_FEATURE - X is any amino acid
  REGION 16..17: note = MISC_FEATURE - X is any amino acid
  REGION 20..21: note = MISC_FEATURE - X is any amino acid
  SITE 24: note = MISC_FEATURE - X is any amino acid
  SITE 28: note = MISC_FEATURE - X is any amino acid
  SITE 40: note = MISC_FEATURE - X is any amino acid
  REGION 43..237: note = MISC_FEATURE - X is any amino acid
XXXXXXXXXXEXXQEXXARXXLQXAQLXGTQVIFTTRPGXLRXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 54 - protein, 100 aa, synthetic construct
  REGION 1..100: note = Synthetic
  REGION 1..64: note = MISC_FEATURE - X is any amino acid
  REGION 66..71: note = MISC_FEATURE - X is any amino acid
  SITE 74: note = MISC_FEATURE - X is any amino acid
  SITE 76: note = MISC_FEATURE - X is any amino acid
  SITE 80: note = MISC_FEATURE - X is any amino acid
XXXXXXXXXXXXXXXXXXXXXXXXXXXXXXXXXXXXXXXXXXXXXXXXXXXXXXXXXXXXXXXXRXXXXXXGQXQXVVRXATEVVTELGKLGIRVTVQLG

SEQ ID NO: 55 - protein, 220 aa, synthetic construct
  REGION 1..220: note = Synthetic
  REGION 1..10: note = MISC_FEATURE - X is any amino acid
  SITE 13: note = MISC_FEATURE - X is any amino acid
  SITE 24: note = MISC_FEATURE - X is any amino acid
  SITE 28: note = MISC_FEATURE - X is any amino acid
  REGION 39..40: note = MISC_FEATURE - X is any amino acid
  REGION 42..91: note = MISC_FEATURE - X is any amino acid
  REGION 93..94: note = MISC_FEATURE - X is any amino acid
  REGION 96..220: note = MISC_FEATURE - X is any amino acid
XXXXXXXXXXDKXTKLIATAVIHXAGRXGTTVHFQGHDXXLXXXXXXXXXXXXXXXXXXXXXXXXXXXXXXXXXXXXXXXXXXXXXXXXXXMXXEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 56 - protein, 29 aa, synthetic construct
  REGION 1..29: note = Synthetic
  SITE 3: note = MISC_FEATURE - X is any amino acid
  SITE 7: note = MISC_FEATURE - X is any amino acid
  REGION 10..11: note = MISC_FEATURE - X is any amino acid
  REGION 13..15: note = MISC_FEATURE - X is any amino acid
  REGION 17..20: note = MISC_FEATURE - X is any amino acid
  SITE 22: note = MISC_FEATURE - X is any amino acid
IKXQRQXYRXXRXXXKXXXXEXEIEVEGD

SEQ ID NO: 57 - protein, 29 aa, synthetic construct
  REGION 1..29: note = Synthetic
  SITE 3: note = MISC_FEATURE - X is ay amino acid
  SITE 7: note = MISC_FEATURE - X is ay amino acid
  REGION 10..11: note = MISC_FEATURE - X is ay amino acid
  REGION 13..15: note = MISC_FEATURE - X is ay amino acid
  REGION 17..20: note = MISC_FEATURE - X is ay amino acid
  SITE 22: note = MISC_FEATURE - X is ay amino acid
IKXMRQXYRXXRXXXKXXXXEXEIEVEGD

SEQ ID NO: 58 - protein, 29 aa, synthetic construct
  REGION 1..29: note = Synthetic
  SITE 3: note = MISC_FEATURE - X is any amino acid
  SITE 7: note = MISC_FEATURE - X is any amino acid
  REGION 10..11: note = MISC_FEATURE - X is any amino acid
  REGION 13..15: note = MISC_FEATURE - X is any amino acid
  REGION 17..20: note = MISC_FEATURE - X is any amino acid
  SITE 22: note = MISC_FEATURE - X is any amino acid
IKXQVQXYRXXRXXXKXXXXEXEIEVEGD

SEQ ID NO: 59 - protein, 29 aa, synthetic construct
  REGION 1..29: note = Synthetic
  SITE 3: note = MISC_FEATURE - X is any amino acid
  SITE 7: note = MISC_FEATURE - X is any amino acid
  REGION 10..11: note = MISC_FEATURE - X is any amino acid
  REGION 13..15: note = MISC_FEATURE - X is any amino acid
  REGION 17..20: note = MISC_FEATURE - X is any amino acid
  SITE 22: note = MISC_FEATURE - X is any amino acid
IKXMVQXYRXXRXXXKXXXXEXEIEVEGD

SEQ ID NO: 60 - protein, 34 aa, synthetic construct
  REGION 1..34: note = Synthetic
  REGION 2..3: note = MISC_FEATURE - X is any amino acid
  SITE 7: note = MISC_FEATURE - X is any amino acid
  SITE 10: note = MISC_FEATURE - X is any amino acid
  SITE 14: note = MISC_FEATURE - X is any amino acid
  REGION 17..20: note = MISC_FEATURE - X is any amino acid
  REGION 29..33: note = MISC_FEATURE - X is any amino acid
EXXQEQXLEXVLRXAEXXXXRVRIRFKGXXXXXV

SEQ ID NO: 61 - protein, 47 aa, synthetic construct
  REGION 1..47: note = Synthetic
  REGION 2..17: note = MISC_FEATURE - X is any amino acid
  REGION 19..20: note = MISC_FEATURE - X is any amino acid
  REGION 23..24: note = MISC_FEATURE - X is any amino acid
  REGION 38..41: note = MISC_FEATURE - X is any amino acid
  SITE 45: note = MISC_FEATURE - X is any amino acid
KXXXXXXXXXXXXXXXXRXXVHXXLRKQGVVAVTQKHXXXXTIYXTE

SEQ ID NO: 62 - protein, 33 aa, synthetic construct
  REGION 1..33: note = Synthetic
  REGION 2..3: note = MISC_FEATURE - X is any amino acid
  SITE 5: note = MISC_FEATURE - X is any amino acid
  REGION 7..8: note = MISC_FEATURE - X is any amino acid
  SITE 20: note = MISC_FEATURE - X is any amino acid
  SITE 23: note = MISC_FEATURE - X is any amino acid
  SITE 27: note = MISC_FEATURE - X is any amino acid
  SITE 31: note = MISC_FEATURE - X is any amino acid
VXXVXIXXRKTEIEVQGIDXRLXKIIXEVIXEE

SEQ ID NO: 63 - protein, 36 aa, synthetic construct
  REGION 1..36: note = Synthetic
  REGION 2..3: note = MISC_FEATURE - X is any amino acid
  REGION 6..7: note = MISC_FEATURE - X is any amino acid
  REGION 10..11: note = MISC_FEATURE - X is any amino acid
  SITE 14: note = MISC_FEATURE - X is any amino acid
  REGION 17..18: note = MISC_FEATURE - X is any amino acid
  REGION 29..33: note = MISC_FEATURE - X is any amino acid
  SITE 35: note = MISC_FEATURE - X is any amino acid
KXXQEXXLKXXLKXANXXGVNVHISFRGXXXXXRXR

SEQ ID NO: 64 - protein, 55 aa, synthetic construct
  REGION 1..55: note = Synthetic
  SITE 3: note = MISC_FEATURE - X is any amino acid
  SITE 7: note = MISC_FEATURE - X is any amino acid
  SITE 10: note = MISC_FEATURE - X is any amino acid
  SITE 18: note = MISC_FEATURE - X is any amino acid
  REGION 29..31: note = MISC_FEATURE - X is any amino acid
  REGION 34..43: note = MISC_FEATURE - X is any amino acid
  REGION 45..47: note = MISC_FEATURE - X is any amino acid
  REGION 49..50: note = MISC_FEATURE - X is any amino acid
  REGION 53..54: note = MISC_FEATURE - X is any amino acid
PEXAKQXLRXVSQTAHKXGVTVTLTFHGXXXFIXXXXXXXXXXQXXXQXXIQXXA

SEQ ID NO: 65 - protein, 36 aa, synthetic construct
  REGION 1..36: note = Synthetic
  REGION 2..3: note = MISC_FEATURE - X is any amino acid
  SITE 7: note = MISC_FEATURE - X is any amino acid
  SITE 10: note = MISC_FEATURE - X is any amino acid
  SITE 14: note = MISC_FEATURE - X is any amino acid
  SITE 18: note = MISC_FEATURE - X is any amino acid
  REGION 30..33: note = MISC_FEATURE - X is any amino acid
  SITE 35: note = MISC_FEATURE - X is any amino acid
EXXQQKXAEXVLEXALRXGTGVTFTTRQGXLQXQXH

SEQ ID NO: 66 - protein, 59 aa, synthetic construct
  REGION 1..59: note = Synthetic
  REGION 2..3: note = MISC_FEATURE - X is any amino acid
  REGION 6..7: note = MISC_FEATURE - X is any amino acid
  REGION 10..11: note = MISC_FEATURE - X is any amino acid
  SITE 14: note = MISC_FEATURE - X is any amino acid
  REGION 17..18: note = MISC_FEATURE - X is any amino acid
  REGION 29..40: note = MISC_FEATURE - X is any amino acid
  REGION 42..43: note = MISC_FEATURE - X is any amino acid
  SITE 46: note = MISC_FEATURE - X is any amino acid
  REGION 49..50: note = MISC_FEATURE - X is any amino acid
  SITE 53: note = MISC_FEATURE - X is any amino acid
  REGION 56..58: note = MISC_FEATURE - X is any amino acid
PXXIKXXMQXXIKXAKXXGVTVTITVSGXXXXXXXXXXXXDXXQEXARXXVQXIAXXXQ

SEQ ID NO: 67 - protein, 36 aa, synthetic construct
  REGION 1..36: note = Synthetic
  SITE 3: note = MISC_FEATURE - X is any amino acid
  REGION 10..11: note = MISC_FEATURE - X is any amino acid
  SITE 20: note = MISC_FEATURE - X is any amino acid
  SITE 29: note = MISC_FEATURE - X is any amino acid
  SITE 31: note = MISC_FEATURE - X is any amino acid
  SITE 33: note = MISC_FEATURE - X is any amino acid
  SITE 35: note = MISC_FEATURE - X is any amino acid
WKXAELIMLXXYDIAQQEGXDVTFSFKEXEXQXTXK

SEQ ID NO: 68 - protein, 55 aa, synthetic construct
  REGION 1..55: note = Synthetic
  REGION 5..12: note = MISC_FEATURE - X is any amino acid
  REGION 23..24: note = MISC_FEATURE - X is any amino acid
  REGION 26..28: note = MISC_FEATURE - X is any amino acid
  REGION 30..47: note = MISC_FEATURE - X is any amino acid
  SITE 49: note = MISC_FEATURE - X is any amino acid
TIIIXXXXXXXXRQTKYLILAPXXEXXXHXXXXXXXXXXXXXXXXXXKXTSGGTT

SEQ ID NO: 69 - protein, 93 aa, synthetic construct
  REGION 1..93: note = Synthetic
  REGION 10..52: note = MISC_FEATURE - X is any amino acid
  REGION 54..55: note = MISC_FEATURE - X is any amino acid
  REGION 62..63: note = MISC_FEATURE - X is any amino acid
  REGION 65..91: note = MISC_FEATURE - X is any amino acid
PHQFYVYQIXXXXXXXXXXXXXXXXXXXXXXXXXXXXXXQXXXXXXXXXEXXAXXIGIYIMXXLXXXXXXXXXXXXXXXXKLIKGFIEHXXRE

SEQ ID NO: 70 - protein, 59 aa, synthetic construct
  REGION 1..59: note = Synthetic
  SITE 3: note = MISC_FEATURE - X is any amino acid
  REGION 6..7: note = MISC_FEATURE - X is any amino acid
  SITE 10: note = MISC_FEATURE - X is any amino acid
  SITE 14: note = MISC_FEATURE - X is any amino acid
  REGION 17..18: note = MISC_FEATURE - X is any amino acid
  SITE 20: note = MISC_FEATURE - X is any amino acid
  REGION 29..31: note = MISC_FEATURE - X is any amino acid
  REGION 33..43: note = MISC_FEATURE - X is any amino acid
  REGION 45..47: note = MISC_FEATURE - X is any amino acid
  SITE 49: note = MISC_FEATURE - X is any amino acid
  REGION 53..54: note = MISC_FEATURE - X is any amino acid
  REGION 56..58: note = MISC_FEATURE - X is any amino acid
KQXALXXLRXVHRXAHXXGXTLSITFSGXXXVXXXXXXXXXXXKXXXRXIVKXXAXXXR

SEQ ID NO: 71 - protein, 36 aa, synthetic construct
  REGION 1..36: note = Synthetic
  REGION 2..3: note = MISC_FEATURE - X is any amino acid
  REGION 6..7: note = MISC_FEATURE - X is any amino acid
  SITE 11: note = MISC_FEATURE - X is any amino acid
  SITE 14: note = MISC_FEATURE - X is any amino acid
  SITE 18: note = MISC_FEATURE - X is any amino acid
  SITE 20: note = MISC_FEATURE - X is any amino acid
  REGION 29..31: note = MISC_FEATURE - X is any amino acid
  SITE 33: note = MISC_FEATURE - X is any amino acid
  SITE 35: note = MISC_FEATURE - X is any amino acid
HXXIEXXLYGXLGXAAMXGXEVTFHSERXXXQXEXK

SEQ ID NO: 72 - protein, 48 aa, synthetic construct
  REGION 1..48: note = Synthetic
  REGION 3..18: note = MISC_FEATURE - X is any amino acid
  REGION 20..21: note = MISC_FEATURE - X is any amino acid
  REGION 24..25: note = MISC_FEATURE - X is any amino acid
  REGION 39..42: note = MISC_FEATURE - X is any amino acid
  SITE 44: note = MISC_FEATURE - X is any amino acid
  SITE 46: note = MISC_FEATURE - X is any amino acid
NTXXXXXXXXXXXXXXXXHXXVEXXVQKEGVFVLVSHHXXXXTXQXYE

SEQ ID NO: 73 - protein, 64 aa, synthetic construct
  REGION 1..64: note = Synthetic
  REGION 10..38: note = MISC_FEATURE - X is any amino acid
  SITE 41: note = MISC_FEATURE - X is any amino acid
  REGION 43..47: note = MISC_FEATURE - X is any amino acid
  SITE 49: note = MISC_FEATURE - X is any amino acid
  REGION 51..52: note = MISC_FEATURE - X is any amino acid
  SITE 55: note = MISC_FEATURE - X is any amino acid
  SITE 59: note = MISC_FEATURE - X is any amino acid
  SITE 62: note = MISC_FEATURE - X is any amino acid
SHSFILGQAXXXXXXXXXXXXXXXXXXXXXXXXXXXXXGTXHXXXXXLXFXXAQXIADXILXII

SEQ ID NO: 74 - protein, 59 aa, synthetic construct
  REGION 1..59: note = Synthetic
  REGION 2..3: note = MISC_FEATURE - X is any amino acid
  REGION 6..7: note = MISC_FEATURE - X is any amino acid
  SITE 10: note = MISC_FEATURE - X is any amino acid
  REGION 30..43: note = MISC_FEATURE - X is any amino acid
  REGION 45..47: note = MISC_FEATURE - X is any amino acid
  REGION 49..50: note = MISC_FEATURE - X is any amino acid
  SITE 53: note = MISC_FEATURE - X is any amino acid
  SITE 57: note = MISC_FEATURE - X is any amino acid
PXXAEXXLRXVHRTAKKQGVTVHLVFTGDXXXXXXXXXXXXXXQXXXHXXVRXIAEXLH

SEQ ID NO: 75 - protein, 32 aa, synthetic construct
  REGION 1..32: note = Synthetic
  REGION 2..3: note = MISC_FEATURE - X is any amino acid
  REGION 6..7: note = MISC_FEATURE - X is any amino acid
  REGION 10..11: note = MISC_FEATURE - X is any amino acid
  SITE 14: note = MISC_FEATURE - X is any amino acid
  SITE 18: note = MISC_FEATURE - X is any amino acid
  SITE 30: note = MISC_FEATURE - X is any amino acid
EXXQEXXARXXLQXAQLXGTQVIFTTRPGXLR

SEQ ID NO: 76 - protein, 36 aa, synthetic construct
  REGION 1..36: note = Synthetic
  REGION 2..7: note = MISC_FEATURE - X is any amino acid
  SITE 10: note = MISC_FEATURE - X is any amino acid
  SITE 12: note = MISC_FEATURE - X is any amino acid
  SITE 16: note = MISC_FEATURE - X is any amino acid
RXXXXXXGQXQXVVRXATEVVTELGKLGIRVTVQLG

SEQ ID NO: 77 - protein, 31 aa, synthetic construct
  REGION 1..31: note = Synthetic
  SITE 3: note = MISC_FEATURE - X is any amino acid
  SITE 14: note = MISC_FEATURE - X is any amino acid
  SITE 18: note = MISC_FEATURE - X is any amino acid
  REGION 29..30: note = MISC_FEATURE - X is any amino acid
DKXTKLIATAVIHXAGRXGTTVHFQGHDXXL

SEQ ID NO: 78 - protein, 200 aa, synthetic construct
  REGION 1..200: note = Synthetic
YEDECEEKARRVAEKVERLKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEDEIAEIVARVISEVIRTLKESGSSYEVIKECVQRIVEEIVEALKRSGTSEDEINEIVRRVKSEVERTLKESGSS

SEQ ID NO: 79 - protein, 180 aa, synthetic construct
  REGION 1..180: note = Synthetic
DEMKKVMEALKKAVELAKKNNDDEVAREIERAAKEIVEALRENNSDEMAKVMLALAKAVLLAAKNNDDEVAREIARAAAEIVEALRENNSDEMAKVMLALAKAVLLAAKNNDDEVAREIARAAAEIVEALRENNSDEMAKKMLELAKRVLDAAKNNDDETAREIARQAAEEVEADRENNS

SEQ ID NO: 80 - protein, 200 aa, synthetic construct
  REGION 1..200: note = Synthetic
YEDEAEEKARRVAEKVERLKRSGTSEDEIAEEVAREISEVIRTLKESGSSYEVIAEIVARIVAEIVEALKRSGTSEDEIAEIVARVISEVIRTLKESGSSYEVIAEIVARIVAEIVEALKRSGTSEDEIAEIVARVISEVIRTLKESGSSYEVIKEIVQRIVEEIVEALKRSGTSEDEINEIVRRVKSEVERTLKESGSS

SEQ ID NO: 81 - protein, 200 aa, synthetic construct
  REGION 1..200: note = Synthetic
SSEKEELRERLVKIVVENAKRKGDDTEEAREAAREAFELVREAAERAGIDSSEVLELAIRLIKEVVENAQREGYDISEAARAAAEAFKRVAEAAKRAGITSSEVLELAIRLIKEVVENAQREGYDISEAARAAAEAFKRVAEAAKRAGITSSETLKRAIEEIRKRVEEAQREGNDISEAARQAAEEFRKKAEELKRRGDV

SEQ ID NO: 82 - protein, 160 aa, synthetic construct
  REGION 1..160: note = Synthetic
SEEVNERVKQLAEKAKEATDKEEVIEIVKELAELAKQSTDSELVNEIVKQLAEVAKEATDKELVIYIVKILAELAKQSTDSELVNEIVKQLAEVAKEATDKELVIYIVKILAELAKQSTDSELVNEIVKQLEEVAKEATDKELVEHIEKILEELKKQSTD

SEQ ID NO: 83 - protein, 152 aa, synthetic construct
  REGION 1..152: note = Synthetic
SEKEKVEELAQRIREQLPDTELAREAQELADEARKSDDSEALKVVYLALRIVQQLPDTELAREALELAKEAVKSTDSEALKVVYLALRIVQQLPDTELAREALELAKEAVKSTDQEALKSVYEALQRVQDKPNTEEARESLERAKEDVKSTD

SEQ ID NO: 84 - protein, 172 aa, synthetic construct
  REGION 1..172: note = Synthetic
CEDRKEKIRELERKARENTGSDEARQAVKEIARIAKEALEEGCCDTAKEAIQRLEDLARDYSGSDVASLAVKAIAKIAETALRNGCCDTAKEAIQRLEDLARDYSGSDVASLAVKAIAKIAETALRNGCKETAEEAIKRLRELAEDYKGSEVAKLAEEAIERIEKVSRERGQ

SEQ ID NO: 85 - protein, 172 aa, synthetic construct
  REGION 1..172: note = Synthetic
NDEKEKLKELLKRAEELAKSPDPEDLKEAVRLAEEVVRERPGSNLAKKALEIILRAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 86 - protein, 172 aa, synthetic construct
  REGION 1..172: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVEVAVKAARVAVEAAKQAGDQDVLRKVSEQAERISKEAKKQGNSEVSEEARKVADEAKKQTGD

SEQ ID NO: 87 - protein, 228 aa, synthetic construct
  REGION 1..228: note = Synthetic
PEDELKRVEKLVKEAEELLRQAKEKGSEEDLEKALRTAEEAAREAKKVLEQAEKEGDPEVALRAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGD

SEQ ID NO: 88 - protein, 220 aa, synthetic construct
  REGION 1..220: note = Synthetic
PELEEWIRRAKEVAKEVEKVAQRAEEEGNPDLRDSAKELRRAVEEAIEEAKKQGNPELVEWVARAAKVAAEVIKVAIQAEKEGNRDLFRAALELVRAVIEAIEEAVKQGNPELVEWVARAAKVAAEVIKVAIQAEKEGNRDLFRAALELVRAVIEAIEEAVKQGNPELVERVARLAKKAAELIKRAIRAEKEGNRDERREALERVREVIERIEELVRQGN

SEQ ID NO: 89 - protein, 232 aa, synthetic construct
  REGION 1..232: note = Synthetic
DEEVQEAVERAEELREEAEELIKKARKTGDPELLRKALEALEEAVRAVEEAIKRNPDNDEAVETAVRLARELKKVAEELQERAKKTGDPELLKLALRALEVAVRAVELAIKSNPDNDEAVETAVRLARELKKVAEELQERAKKTGDPELLKLALRALEVAVRAVELAIKSNPDNEEAVETAKRLAEELRKVAELLEERAKETGDPELQELAKRAKEVADRARELAKKSNPNN

SEQ ID NO: 90 - protein, 390 aa, synthetic construct
  REGION 1..390: note = Synthetic
GNTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSELAKKALEIILRAAEELAKLPDPEALHEAVRAAEHVVRSQPGSEAAKEALRIIQEAAELLKESPDPTAIIRAARALLKIARTTGDEEAAKEAIEAAKKAADLARERGDDELVCEALALLVAAQVELLKQQGTSAVEIAKIVARVISEVIRTLKEKGSSYEVICECVARIVAEIVEALKRSGTSAAIIALIVALVISEVIRTLKESGSSFEVILECVIRIVLEIIEALKRSGTSEQDVMLIVMAVLLVVLATLQLSGSGSLEHHHHHH

SEQ ID NO: 91 - protein, 379 aa, synthetic construct
  REGION 1..379: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSELAKKALEIILRAAEELAKLPDPEALHEAVRAAEHVVRSQPGSEAAKEALRIIQEAAELLKESPDPTAIIRAARALLKIARTTGDEEAAKEAIEAAKKAADLARERGDDELVCEALALLVAAQVELLKQQGTSAVEIAKIVARVISEVIRTLKEKGSSYEVICECVARIVAEIVEALKRSGTSAAIIALIVALVISEVIRTLKESGSSFEVILECVIRIVLEIIEALKRSGTSEQDVMLIVMAVLLVVLATLQLSGS

SEQ ID NO: 92 - protein, 296 aa, synthetic construct
  REGION 1..296: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSHHWGSGSHHHHHH

SEQ ID NO: 93 - protein, 281 aa, synthetic construct
  REGION 1..281: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 94 - protein, 300 aa, synthetic construct
  REGION 1..300: note = Synthetic
MTWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGSGGSGSHHWGLEHHHHHH

SEQ ID NO: 95 - protein, 281 aa, synthetic construct
  REGION 1..281: note = Synthetic
TWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 96 - protein, 339 aa, synthetic construct
  REGION 1..339: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSHHWGSGSHHHHHH

SEQ ID NO: 97 - protein, 324 aa, synthetic construct
  REGION 1..324: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 98 - protein, 296 aa, synthetic construct
  REGION 1..296: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSHHWGSGSHHHHHH

SEQ ID NO: 99 - protein, 281 aa, synthetic construct
  REGION 1..281: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 100 - protein, 339 aa, synthetic construct
  REGION 1..339: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSHHWGSGSHHHHHH

SEQ ID NO: 101 - protein, 324 aa, synthetic construct
  REGION 1..324: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 102 - protein, 339 aa, synthetic construct
  REGION 1..339: note = Synthetic
NTHFIVVHGTEEARQLAEEIVRLIAEALGTEVRFEKKDGLLSIEVKNLPEEAQRLIQKLLQLAVRIAQAAKSGDNDQLRELAEDALRLAKEAEKLGDLGAAAVAARLAVEAAKQAGDNDVLRKVAEQALRIAKEAEKQGLVDVAVEAARVAVEAAKQAGDNDVLRKVAEQALRIAKEAEKQGNVEVAVKAARVAVEAAKQAGDNDVLRKVAEQALRIVREALKQGNKEVAKKALEVAIEAANQAGDQKLLAEILLLAIEVLVVEMGVTMETHKSGNKVKVVIKGLHESQQETLRKLVHELLRKLGVVAVTQKHGDTVTIYVTEGGSHHWGSGSHHHHHH

SEQ ID NO: 103 - protein, 323 aa, synthetic construct
  REGION 1..323: note = Synthetic
NTHFIVVHGTEEARQLAEEIVRLIAEALGTEVRFEKKDGLLSIEVKNLPEEAQRLIQKLLQLAVRIAQAAKSGDNDQLRELAEDALRLAKEAEKLGDLGAAAVAARLAVEAAKQAGDNDVLRKVAEQALRIAKEAEKQGLVDVAVEAARVAVEAAKQAGDNDVLRKVAEQALRIAKEAEKQGNVEVAVKAARVAVEAAKQAGDNDVLRKVAEQALRIVREALKQGNKEVAKKALEVAIEAANQAGDQKLLAEILLLAIEVLVVEMGVTMETHKSGNKVKVVIKGLHESQQETLRKLVHELLRKLGVVAVTQKHGDTVTIYVTE

SEQ ID NO: 104 - protein, 359 aa, synthetic construct
  REGION 1..359: note = Synthetic
NTHFIVVHGTEEARQLAEEIVRLIAEALGTEVRFEKKDGLLSIEVKNLPEEAQRLIQKLLQLAVRIAAAAESGDNDQLRELAEDALRLAEEAEKLGDLGAAAVAARLAVEAAKQAGDNDVLREVAEQALEIAKEAEKQGLVDVAVEAARVAVEAAKQAGDNDVLRKVAEQALRIAKEAEKQGNVEVAVKAARVAVEAAKQAGDNDVLRKVAEQALRIAKEAEKQGNVEVAALALVVAVNAAQAAGDQDLLRKIAEQAERLAKLAEKQGRRDVALLASIIALVAKMGVPMEVHPSGNEVKVVIKGLHKSQQEQLLKEVLKAANKLGVNVHISFRGDTVTIRVRGGGSHHWGSGSHHHHHH

SEQ ID NO: 105 - protein, 343 aa, synthetic construct
  REGION 1..343: note = Synthetic
NTHFIVVHGTEEARQLAEEIVRLIAEALGTEVRFEKKDGLLSIEVKNLPEEAQRLIQKLLQLAVRIAAAAESGDNDQLRELAEDALRLAEEAEKLGDLGAAAVAARLAVEAAKQAGDNDVLREVAEQALEIAKEAEKQGLVDVAVEAARVAVEAAKQAGDNDVLRKVAEQALRIAKEAEKQGNVEVAVKAARVAVEAAKQAGDNDVLRKVAEQALRIAKEAEKQGNVEVAALALVVAVNAAQAAGDQDLLRKIAEQAERLAKLAEKQGRRDVALLASIIALVAKMGVPMEVHPSGNEVKVVIKGLHKSQQEQLLKEVLKAANKLGVNVHISFRGDTVTIRVRG

SEQ ID NO: 106 - protein, 547 aa, synthetic construct
  REGION 1..547: note = Synthetic
HHHHHHGSGSNTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGLVDVAVEAARVAVEAAKQAGDNDVLRKVAEQALRIAKEAEKQGNVEVAIKALEVAAEAAAQAGDKDVLKKILEQLERLAELAKKQGNKELAIKIFELFIKVIVALMGVRMLSHKGGNAVIVVIEGLHPSQAEQLLRLVHRIAKKAGVTVHLVFTGDIVVIMVVVGASEEEQEDMHRLVREIAEALHFAKSFGADEKALELLLKALLALLELVVASKEGDEEEFRKLAEKALELAKQLVELAKKLGIAALVLLAARIALKVAELAAKNGDKEVFKKAAESALEVAKRLVEVASKEGDAELVLEAAKVALRVAELAAKNGDKEVFKKAAESALEVAKRLVEVASKEGDAELVEEAAKVAEEVRKLAKKQGDEEVYEKARETAREVKEELKRVREEKGDGS

SEQ ID NO: 107 - protein, 535 aa, synthetic construct
  REGION 1..535: note = Synthetic
NTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGLVDVAVEAARVAVEAAKQAGDNDVLRKVAEQALRIAKEAEKQGNVEVAIKALEVAAEAAAQAGDKDVLKKILEQLERLAELAKKQGNKELAIKIFELFIKVIVALMGVRMLSHKGGNAVIVVIEGLHPSQAEQLLRLVHRIAKKAGVTVHLVFTGDIVVIMVVVGASEEEQEDMHRLVREIAEALHFAKSFGADEKALELLLKALLALLELVVASKEGDEEEFRKLAEKALELAKQLVELAKKLGIAALVLLAARIALKVAELAAKNGDKEVFKKAAESALEVAKRLVEVASKEGDAELVLEAAKVALRVAELAAKNGDKEVFKKAAESALEVAKRLVEVASKEGDAELVEEAAKVAEEVRKLAKKQGDEEVYEKARETAREVKEELKRVREEKGD

SEQ ID NO: 108 - protein, 410 aa, synthetic construct
  REGION 1..410: note = Synthetic
HHHHHHGSGSGRQEKVLKSIEETVRKMGVEMLTFRAGKAVIVVIRGLHPEQAKQLLRDVSQTAHKQGVTVTLTFHGDVVFILVLVGASEEQQRAMQLLIQALARIIHEAKRRGVSEEQLKRMIEAAARLIEVLLKALEAAREGNTDEVREQLQRALEIVREIGLTAAVRLALLVVEAVATLAAKRGNTDAVREALEVALEIARESGTTEAVKLALEVVARVAIEAARRGNTDAVREALEVALEIARESGTTEAVKLALEVVARVAIEAARRGNTDAVREALAVAVKIALKSGTEEAFRLAKEVIKRVSDEAKKQGNEDAVKEAESFDAAAELILSLLKLFRELHERGTEIVVEVHINGRKTEIEVQGIDKRLLQIILEVIIEEIAREGPDKVEVNVHSGGQTWTFRYGGS

SEQ ID NO: 109 - protein, 398 aa, synthetic construct
  REGION 1..398: note = Synthetic
GRQEKVLKSIEETVRKMGVEMLTFRAGKAVIVVIRGLHPEQAKQLLRDVSQTAHKQGVTVTLTFHGDVVFILVLVGASEEQQRAMQLLIQALARIIHEAKRRGVSEEQLKRMIEAAARLIEVLLKALEAAREGNTDEVREQLQRALEIVREIGLTAAVRLALLVVEAVATLAAKRGNTDAVREALEVALEIARESGTTEAVKLALEVVARVAIEAARRGNTDAVREALEVALEIARESGTTEAVKLALEVVARVAIEAARRGNTDAVREALAVAVKIALKSGTEEAFRLAKEVIKRVSDEAKKQGNEDAVKEAESFDAAAELILSLLKLFRELHERGTEIVVEVHINGRKTEIEVQGIDKRLLQIILEVIIEEIAREGPDKVEVNVHSGGQTWTFRYG

SEQ ID NO: 110 - protein, 357 aa, synthetic construct
  REGION 1..357: note = Synthetic
HHHHHHGSGSPHQFYVYQIDEHVAQLIEKFVRDISRREGTEVRFEKRDGQLEIEVKNLHEAQAIAIGIYIMILLLHQSGTSEDEIAEEIAKLIKGFIEHLKREGSSYEVICEAVAAAVAAIVKALKGCGTSEDEIAEIVARVISEVIRTLKESGSSYEVICECVARIVAEIVQALKESGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRGGS

SEQ ID NO: 111 - protein, 345 aa, synthetic construct
  REGION 1..345: note = Synthetic
PHQFYVYQIDEHVAQLIEKFVRDISRREGTEVRFEKRDGQLEIEVKNLHEAQAIAIGIYIMILLLHQSGTSEDEIAEEIAKLIKGFIEHLKREGSSYEVICEAVAAAVAAIVKALKGCGTSEDEIAEIVARVISEVIRTLKESGSSYEVICECVARIVAEIVQALKESGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRG

SEQ ID NO: 112 - protein, 352 aa, synthetic construct
  REGION 1..352: note = Synthetic
TVTFDITNIDDKSTKLIATAVIHIAGREGTTVHFQGHDGQLEIEVKNLHEKWKRLIEMLIEAARRAQDPDPESLKEAVRIAEELVRLHPGNMLAEAALKVILTAAEELAKLPDPEALKEAVKAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVEREQPGSELAVVAAIISAVARMGVTMELHPSGNEVKVVIKGLHIKQQRQLYRDVREAAKKAGVEVEIEVEGDTVTIVVRGGGSHHWGSGSHHHHHH

SEQ ID NO: 113 - protein, 336 aa, synthetic construct
  REGION 1..336: note = Synthetic
TVTFDITNIDDKSTKLIATAVIHIAGREGTTVHFQGHDGQLEIEVKNLHEKWKRLIEMLIEAARRAQDPDPESLKEAVRIAEELVRLHPGNMLAEAALKVILTAAEELAKLPDPEALKEAVKAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVEREQPGSELAVVAAIISAVARMGVTMELHPSGNEVKVVIKGLHIKQQRQLYRDVREAAKKAGVEVEIEVEGDTVTIVVRG

SEQ ID NO: 114 - protein, 362 aa, synthetic construct
  REGION 1..362: note = Synthetic
GSSIFLLSNVSEDAAQLAEELVREISKKEGTEVRFEKDDGFLTIEVKNLSEERLREIAKALQLIVDVANAERVVRERPGSNLAKKALEIILRAAEELAKLDLKASLKAAVRAALKVVAEQPGSNLAKKALEIIQRAAEELAKLPDPEAQKEAQLAAELVRAAELAKSPDPEDLKEAVRLAEEVVRERPGSNLAKAALAIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGGSWGLEHHHHHH

SEQ ID NO: 115 - protein, 396 aa, synthetic construct
  REGION 1..396: note = Synthetic
TWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKAQEIILRAAEELAKLPDPEAQKEAAKAIARRVAAKVERLKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRG

SEQ ID NO: 116 - protein, 408 aa, synthetic construct
  REGION 1..408: note = Synthetic
GTWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKAMEIILRAAEELAKLPDPEAQKEAAKAIARRVAAKVEELKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRGGGSLEHHHHHH

SEQ ID NO: 117 - protein, 396 aa, synthetic construct; identical to SEQ ID NO: 115
  REGION 1..396: note = Synthetic

SEQ ID NO: 118 - protein, 569 aa, synthetic construct
  REGION 1..569: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSGSGSGSSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 119 - protein, 324 aa, synthetic construct; identical to SEQ ID NO: 97
  REGION 1..324: note = Synthetic

SEQ ID NO: 120 - protein, 578 aa, synthetic construct
  REGION 1..578: note = Synthetic
MSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGSGSGSTTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGLEHHHHHH

SEQ ID NO: 121 - protein, 324 aa, synthetic construct; identical to SEQ ID NO: 97
  REGION 1..324: note = Synthetic

SEQ ID NO: 122 - protein, 264 aa, synthetic construct
  REGION 1..264: note = Synthetic
HHHHHHGSGLNDIFEAQKIEWHEAENLYFQSGSSSEKEELRERLVKIVVENAKRKGDDTEEAREAAREAFELVREAAERAGIDSSEVLELAIRLIKEVVENAQREGYDISEAARAAAEAFKRVAEAAKRAGITSSEVLELAIILIKLVVELAQRKGYDISEAARAAAELFKRLAEALKRAGKTSERALALLILLLAIEILVRDMGVTMETHPSGNEVKVVIKGLHIKQQRQLYRLVREAAKLLGVEVEIEVEGDTVTIVVRGGS

SEQ ID NO: 123 - protein, 229 aa, synthetic construct
  REGION 1..229: note = Synthetic
SSEKEELRERLVKIVVENAKRKGDDTEEAREAAREAFELVREAAERAGIDSSEVLELAIRLIKEVVENAQREGYDISEAARAAAEAFKRVAEAAKRAGITSSEVLELAIILIKLVVELAQRKGYDISEAARAAAELFKRLAEALKRAGKTSERALALLILLLAIEILVRDMGVTMETHPSGNEVKVVIKGLHIKQQRQLYRLVREAAKLLGVEVEIEVEGDTVTIVVRG

SEQ ID NO: 124 - protein, 218 aa, synthetic construct
  REGION 1..218: note = Synthetic
HHHHHHGSGLNDIFEAQKIEWHEAENLYFQSGSSEKEKVEELAQRIREQLPDTELAREAQELADEARKSDDSEALKVVYLALRIVQQLPDTELAREALELAKEAVKSTDSEKLKVVYLALRVVQQLPDTEEARKALEIAKEAVKADAQILLAIARAVLKMGVEMEVHPSGNEVKVVIKGLHIKQQRQLYRDVREAAKKAGVEVEIEVEGDTVTIVVRE

SEQ ID NO: 125 - protein, 185 aa, synthetic construct
  REGION 1..185: note = Synthetic
SEKEKVEELAQRIREQLPDTELAREAQELADEARKSDDSEALKVVYLALRIVQQLPDTELAREALELAKEAVKSTDSEKLKVVYLALRVVQQLPDTEEARKALEIAKEAVKADAQILLAIARAVLKMGVEMEVHPSGNEVKVVIKGLHIKQQRQLYRDVREAAKKAGVEVEIEVEGDTVTIVVRE

SEQ ID NO: 126 - protein, 269 aa, synthetic construct
  REGION 1..269: note = Synthetic
HHHHHHGSGLNDIFEAQKIEWHEAENLYFQSGSCEDRKEKIRELERKARENTGSDEARQAVKEIARIAKEALEEGCCDTAKEAIQRLEDLARDYSGSDVASLAVKAIAKIAETALRNGCCDTAKEAIQRLEDLARDYSGSDVASLAVEAILRIALIALANGCEETAEEARKRLRELAEDYKGSEVAKLAESAERLIEILKIIAKTVRKMGVTMDVRPSGTEVEVVIKGLHIKQQRQLYRDVREAAKKLGVEVEIEVEGDTVTIVVRGGS

SEQ ID NO: 127 - protein, 234 aa, synthetic construct
  REGION 1..234: note = Synthetic
CEDRKEKIRELERKARENTGSDEARQAVKEIARIAKEALEEGCCDTAKEAIQRLEDLARDYSGSDVASLAVKAIAKIAETALRNGCCDTAKEAIQRLEDLARDYSGSDVASLAVEAILRIALIALANGCEETAEEARKRLRELAEDYKGSEVAKLAESAERLIEILKIIAKTVRKMGVTMDVRPSGTEVEVVIKGLHIKQQRQLYRDVREAAKKLGVEVEIEVEGDTVTIVVRG

SEQ ID NO: 128 - protein, 235 aa, synthetic construct
  REGION 1..235: note = Synthetic
HHHHHHGSGLNDIFEAQKIEWHEAENLYFQSGSNDEKEKLKELLKRAEELAKSPDPEDLKEAVRLAEEVVRERPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 129 - protein, 202 aa, synthetic construct
  REGION 1..202: note = Synthetic
NDEKEKLKELLKRAEELAKSPDPEDLKEAVRLAEEVVRERPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 130 - protein, 282 aa, synthetic construct
  REGION 1..282: note = Synthetic
HHHHHHGSGLNDIFEAQKIEWHEAENLYFQSGSYEDECEEKARRVAEKVERLKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRG

SEQ ID NO: 131 - protein, 249 aa, synthetic construct
  REGION 1..249: note = Synthetic
YEDECEEKARRVAEKVERLKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEEEIAEIVARVIQEVIRTLKESGSSYEVIRECLRRILEEVIEALKRSGVDSSEIVLIIIKIAVAVMGVTMEEHRSGNEVKVVIKGLHESQQEELLELVLRAAELAGVRVRIRFKGDTVTIVVRG

SEQ ID NO: 132 - protein, 242 aa, synthetic construct
  REGION 1..242: note = Synthetic
HHHHHHGSGLNDIFEAQKIEWHEAENLYFQSGSDEMKKVMEALKKAVELAKKNNDDEVAREIERAAKEIVEALRENNSDEMAKVMLALAKAVLLAAKNNDDEVAREIARAAAEIVEALRENNLEVMALVARLLAEAVLLAAKNNDDEVAREIAREAAEIVEKLRENNDATMAVRAAIRAAVIRMGVTMEEHRSGNEVKVVIKGLHESQQEELLEIVLRAAELAGVRVRIRFKGDTVTIVVRG

SEQ ID NO: 133 - protein, 209 aa, synthetic construct
  REGION 1..209: note = Synthetic
DEMKKVMEALKKAVELAKKNNDDEVAREIERAAKEIVEALRENNSDEMAKVMLALAKAVLLAAKNNDDEVAREIARAAAEIVEALRENNLEVMALVARLLAEAVLLAAKNNDDEVAREIAREAAEIVEKLRENNDATMAVRAAIRAAVIRMGVTMEEHRSGNEVKVVIKGLHESQQEELLEIVLRAAELAGVRVRIRFKGDTVTIVVRG

SEQ ID NO: 134 - protein, 254 aa, synthetic construct
  REGION 1..254: note = Synthetic
HHHHHHGSGLNDIFEAQKIEWHEAENLYFQSGSSEEVNERVKQLAEKAKEATDKEEVIEIVKELAELAKQSTDSELVNEIVKQLAEVAKEATDKELVIYIVKILAELAKQSTDSELVNEIVKQLAEVAKEATDKELVIYIVDILLKLAEQADDDELVEEIRKQLEEVAKEATDKELVEIIKAVIVLLVIISVVARMGVTMEIHKSGREVKVVIKGLHESQQEQLLEAVLRAAEEAGVRVRIRFKGDTVTIVVRG

SEQ ID NO: 135 - protein, 221 aa, synthetic construct
  REGION 1..221: note = Synthetic
SEEVNERVKQLAEKAKEATDKEEVIEIVKELAELAKQSTDSELVNEIVKQLAEVAKEATDKELVIYIVKILAELAKQSTDSELVNEIVKQLAEVAKEATDKELVIYIVDILLKLAEQADDDELVEEIRKQLEEVAKEATDKELVEIIKAVIVLLVIISVVARMGVTMEIHKSGREVKVVIKGLHESQQEQLLEAVLRAAEEAGVRVRIRFKGDTVTIVVRG

SEQ ID NO: 136 - protein, 246 aa, synthetic construct
  REGION 1..246: note = Synthetic
HHHHHHGSGLNDIFEAQKIEWHEAENLYFQSGSNDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEALKQGNVDVAAKAAQVAEEAAKQAGDQDVLRKVKEVQIEIVLAAIELTVRKMGVTMETHRSGREVKVVIKGLHESQQEQLLEDVLRIAELAGVRVRIRFKGDTVTIVVRG

SEQ ID NO: 137 - protein, 213 aa, synthetic construct
  REGION 1..213: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEALKQGNVDVAAKAAQVAEEAAKQAGDQDVLRKVKEVQIEIVLAAIELTVRKMGVTMETHRSGREVKVVIKGLHESQQEQLLEDVLRIAELAGVRVRIRFKGDTVTIVVRG

SEQ ID NO: 138 - protein, 287 aa, synthetic construct
  REGION 1..287: note = Synthetic
HHHHHHGSGLNDIFEAQKIEWHEAENLYFQSGSDEEVQEAVERAEELREEAEELIKKARKTGDAELLRKALEALEEAVRAVEEAIKRNPDNDEAVETAVRLARELKKVAEELQERAKKTGDAELLKLALRALEVAVRAVELAIKSNPDNDEAVETAVRLARELAKVAEELIERAKKTGDKELLKLAKRALEVAMRAVSLALKSNPDNEEARRVAAELVLLVIRAAVIEMGVTMEEHRSGNRVKVVIKGLHESQQEQLLEDVLRAAEIAGVRVRIRFKGDTVTIVVEG

SEQ ID NO: 139 - protein, 254 aa, synthetic construct
  REGION 1..254: note = Synthetic
DEEVQEAVERAEELREEAEELIKKARKTGDAELLRKALEALEEAVRAVEEAIKRNPDNDEAVETAVRLARELKKVAEELQERAKKTGDAELLKLALRALEVAVRAVELAIKSNPDNDEAVETAVRLARELAKVAEELIERAKKTGDKELLKLAKRALEVAMRAVSLALKSNPDNEEARRVAAELVLLVIRAAVIEMGVTMEEHRSGNRVKVVIKGLHESQQEQLLEDVLRAAEIAGVRVRIRFKGDTVTIVVEG

SEQ ID NO: 140 - protein, 223 aa, synthetic construct
  REGION 1..223: note = Synthetic
HHHHHHGSGLNDIFEAQKIEWHEAENLYFQSGSSEKEKVEELAQRIREQLPDTELAREAQELADEARKSDDSEALKVVYLALRIVQQLPDTELAREALELAKEAVKSTDPAQLIVVQLALKIVQKLPDTEEARRALELAKEAVKSTNKAELVVIAIELLVLLMGVTMEVHKSGNKVKVVIKGLHESQQEQLRKLVHEALRAAGVVAVTQKHGDTVTIYVTEGS

SEQ ID NO: 141 - protein, 188 aa, synthetic construct
  REGION 1..188: note = Synthetic
SEKEKVEELAQRIREQLPDTELAREAQELADEARKSDDSEALKVVYLALRIVQQLPDTELAREALELAKEAVKSTDPAQLIVVQLALKIVQKLPDTEEARRALELAKEAVKSTNKAELVVIAIELLVLLMGVTMEVHKSGNKVKVVIKGLHESQQEQLRKLVHEALRAAGVVAVTQKHGDTVTIYVTE

SEQ ID NO: 142 - protein, 241 aa, synthetic construct
  REGION 1..241: note = Synthetic
HHHHHHGSGLNDIFEAQKIEWHEAENLYFQSGSNDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIVREALKQGNKEVAKKALEVAIEAANQAGDQKLLSKILQLAIEVLVVEMGVTMETHKSGNKVKVVIKGLHESQQETLRKLVHELLRKLGVVAVTQKHGDTVTIYVTEGS

SEQ ID NO: 143 - protein, 206 aa, synthetic construct
  REGION 1..206: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIVREALKQGNKEVAKKALEVAIEAANQAGDQKLLSKILQLAIEVLVVEMGVTMETHKSGNKVKVVIKGLHESQQETLRKLVHELLRKLGVVAVTQKHGDTVTIYVTE

SEQ ID NO: 144 - protein, 251 aa, synthetic construct
  REGION 1..251: note = Synthetic
GSSVEFHIVNISEKAAQIIERAVRAISKELGTEVRFEKRDGELTIEVKNLHERRLQEILLLIEAVKLLLLALKAVKEDPSTDALRAVLEAVRFASEVAKRVENPEAVAVLAELVIELALEAVKEDPSTDALRAVLEAVRLASEVAKRVTDADKALKIAKLVIELALEAVKEDPSEEAKRAVEEAKRLAEEVSKRVTDPELSEKIRQLVKELEEEAQKEDPSGSHHWGSGLNDIFEAQKIEWHEGSHHHHHH

SEQ ID NO: 145 - protein, 219 aa, synthetic construct
  REGION 1..219: note = Synthetic
SVEFHIVNISEKAAQIIERAVRAISKELGTEVRFEKRDGELTIEVKNLHERRLQEILLLIEAVKLLLLALKAVKEDPSTDALRAVLEAVRFASEVAKRVENPEAVAVLAELVIELALEAVKEDPSTDALRAVLEAVRLASEVAKRVTDADKALKIAKLVIELALEAVKEDPSEEAKRAVEEAKRLAEEVSKRVTDPELSEKIRQLVKELEEEAQKEDPS

SEQ ID NO: 146 - protein, 314 aa, synthetic construct
  REGION 1..314: note = Synthetic
GSSVEFHIVNIDEDVAQLIELAVKLISKEEGTEVRFEKRDGELTIEVKNLHEKDLQLILELIEALLLIARAIELLRQAKEKGSEEDLEKALRTAEESARRLKKVLEKAEKLGNLGVALAAVAGVVLVAELLLRIAKESGSEEALERALRVAEEAARLAKRVLELAEKLGDAEAALLAVELVVRVAELLLRIAKESGSEEALERALRVAEEAARLAKRVLELAEKQGDAEVARRAVELVKRVAELLERIARESGSEEAKERAERVREEARELQERVKELREREGDGSHHWGSGLNDIFEAQKIEWHEGSHHHHHH

SEQ ID NO: 147 - protein, 282 aa, synthetic construct
  REGION 1..282: note = Synthetic
SVEFHIVNIDEDVAQLIELAVKLISKEEGTEVRFEKRDGELTIEVKNLHEKDLQLILELIEALLLIARAIELLRQAKEKGSEEDLEKALRTAEESARRLKKVLEKAEKLGNLGVALAAVAGVVLVAELLLRIAKESGSEEALERALRVAEEAARLAKRVLELAEKLGDAEAALLAVELVVRVAELLLRIAKESGSEEALERALRVAEEAARLAKRVLELAEKQGDAEVARRAVELVKRVAELLERIARESGSEEAKERAERVREEARELQERVKELREREGD

SEQ ID NO: 148 - protein, 279 aa, synthetic construct
  REGION 1..279: note = Synthetic
HHHHHHGSGLNDIFEAQKIEWHEAENLYFQSGSTEDERRELEKVARKAIEAAREGNTDEVREQLQRALEIARESGTTEAVKLALEVVARVAIEAARRGNTDAVREALEVALEIARESGTTEAVKLALEVVARVAIEAARRGNTDAVREALAVAVKIALKSGTEEAFRLAKEVIKRVSDEAKKQGNEDAVKEAESFDAAAELILSLLKLFRELHERGTEIVVEVHINGRKTEIEVQGIDKRLLQIILEVIIEEIAREGPDKVEVNVHSGGQTWTFRYGGS

SEQ ID NO: 149 - protein, 244 aa, synthetic construct
  REGION 1..244: note = Synthetic
TEDERRELEKVARKAIEAAREGNTDEVREQLQRALEIARESGTTEAVKLALEVVARVAIEAARRGNTDAVREALEVALEIARESGTTEAVKLALEVVARVAIEAARRGNTDAVREALAVAVKIALKSGTEEAFRLAKEVIKRVSDEAKKQGNEDAVKEAESFDAAAELILSLLKLFRELHERGTEIVVEVHINGRKTEIEVQGIDKRLLQIILEVIIEEIAREGPDKVEVNVHSGGQTWTFRYG

SEQ ID NO: 150 - protein, 261 aa, synthetic construct
  REGION 1..261: note = Synthetic
HHHHHHGSGLNDIFEAQKIEWHEAENLYFQSGSNDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVEVAALALVVATNAAQAAGDQDLLRKIAEQAERLAKLAKKQGRRDVALLALIIALVSKMGVPMEVHPSGKEVKVVIKGLHKSQQEQLLKLVLKAANKLGVNVHISFRGDTVTIRVRGGS

SEQ ID NO: 151 - protein, 226 aa, synthetic construct
  REGION 1..226: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVEVAALALVVATNAAQAAGDQDLLRKIAEQAERLAKLAKKQGRRDVALLALIIALVSKMGVPMEVHPSGKEVKVVIKGLHKSQQEQLLKLVLKAANKLGVNVHISFRGDTVTIRVRG

SEQ ID NO: 152 - protein, 261 aa, synthetic construct
  REGION 1..261: note = Synthetic
HHHHHHGSGLNDIFEAQKIEWHEAENLYFQSGSNDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVPVAVKALLVALNAAVAAGDQDVLRKISEQAERARKLAEKQGDKLLAFVLALISLVAQMGVPMEIHPSGNEVKVVIKGLHKSQQEQLLKLVLKLANKLGVNVHISFRGDTVTIRVRGGS

SEQ ID NO: 153 - protein, 226 aa, synthetic construct
  REGION 1..226: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVPVAVKALLVALNAAVAAGDQDVLRKISEQAERARKLAEKQGDKLLAFVLALISLVAQMGVPMEIHPSGNEVKVVIKGLHKSQQEQLLKLVLKLANKLGVNVHISFRGDTVTIRVRG

SEQ ID NO: 154 - protein, 284 aa, synthetic construct
  REGION 1..284: note = Synthetic
GSTTNFHLINGSEEARQLIQKAVEAISKKEGTEVHFEKSDGTLEIRVKNLHPRQEDLIKKFIEALLLALVAKGELEQAEKEGDAEVALRAVEKVVRVAELLLRLAKEAGSEEALKAALEIAEQAARLAKRVLELAEKQGDAEVALRAVELVVRVAELLLRIAKESGSEEALERALRVAEEAARLAKRVLELAEKQGDAEVARRAVELVKRVAELLERIARESGSEEAKERAERVREEARELQERVKELREREGDGSHHWGSGLNDIFEAQKIEWHEGSHHHHHH

SEQ ID NO: 155 - protein, 252 aa, synthetic construct
  REGION 1..252: note = Synthetic
TTNFHLINGSEEARQLIQKAVEAISKKEGTEVHFEKSDGTLEIRVKNLHPRQEDLIKKFIEALLLALVAKGELEQAEKEGDAEVALRAVEKVVRVAELLLRLAKEAGSEEALKAALEIAEQAARLAKRVLELAEKQGDAEVALRAVELVVRVAELLLRIAKESGSEEALERALRVAEEAARLAKRVLELAEKQGDAEVARRAVELVKRVAELLERIARESGSEEAKERAERVREEARELQERVKELREREGD

SEQ ID NO: 156 - protein, 279 aa, synthetic construct
  REGION 1..279: note = Synthetic
GSTTNFHLINGSEEARQVIEEIVEIIARLAGTEVHFEKSDGTLEIRVKNLHEELERLIKELIELALLLQLAKKEAIEEAKKQGNPELVEWVARAAEVVKEVLRVAAEAAGAGNPDLAKAAAELARAVIEAIEEAVKQGNAELVEWVARAAKVAAEVIKVAIQAEKEGNRDLFRAALELVRAVIEAIEEAVKQGNAELVERVARLAKKAAELIKRAIRAEKEGNRDERREALERVREVIERIEELVRQGNGSHHWGSGLNDIFEAQKIEWHEGSHHHHHH

SEQ ID NO: 157 - protein, 247 aa, synthetic construct
  REGION 1..247: note = Synthetic
TTNFHLINGSEEARQVIEEIVEIIARLAGTEVHFEKSDGTLEIRVKNLHEELERLIKELIELALLLQLAKKEAIEEAKKQGNPELVEWVARAAEVVKEVLRVAAEAAGAGNPDLAKAAAELARAVIEAIEEAVKQGNAELVEWVARAAKVAAEVIKVAIQAEKEGNRDLFRAALELVRAVIEAIEEAVKQGNAELVERVARLAKKAAELIKRAIRAEKEGNRDERREALERVREVIERIEELVRQGN

SEQ ID NO: 158 - protein, 235 aa, synthetic construct
  REGION 1..235: note = Synthetic
GSNTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGLVDVAVEAARVAVEAAKQAGDQDVLRKVSEQAERISKEAKKQGNSEVSEEARKVADEAKKQTGDGSHHWGSGLNDIFEAQKIEWHEGSHHHHHH

SEQ ID NO: 159 - protein, 203 aa, synthetic construct
  REGION 1..203: note = Synthetic
NTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGLVDVAVEAARVAVEAAKQAGDQDVLRKVSEQAERISKEAKKQGNSEVSEEARKVADEAKKQTGD

SEQ ID NO: 160 - protein, 292 aa, synthetic construct
  REGION 1..292: note = Synthetic
GSNTHFIVVHGGEEARQLAETAVREISKKEGTEVRFEKKDGLLSIEVKNLSEELQRLIQELLQLLVRLAALLEAVRAVEEAIKRNPDNDEAVETAVRLARELKKVAELLQELAKKAGVPAILRGALLALEVAVRAVELAIKSNPDNDEAVETAVRLARELKKVAEELQERAKKTGDAELLKLALRALEVAVRAVELAIKSNPDNEEAVETAKRLAEELRKVAELLEERAKETGDPELQELAKRAKEVADRARELAKKSNPNNGSHHWGSGLNDIFEAQKIEWHEGSHHHHHH

SEQ ID NO: 161 - protein, 260 aa, synthetic construct
  REGION 1..260: note = Synthetic
NTHFIVVHGGEEARQLAETAVREISKKEGTEVRFEKKDGLLSIEVKNLSEELQRLIQELLQLLVRLAALLEAVRAVEEAIKRNPDNDEAVETAVRLARELKKVAELLQELAKKAGVPAILRGALLALEVAVRAVELAIKSNPDNDEAVETAVRLARELKKVAEELQERAKKTGDAELLKLALRALEVAVRAVELAIKSNPDNEEAVETAKRLAEELRKVAELLEERAKETGDPELQELAKRAKEVADRARELAKKSNPNN

SEQ ID NO: 162 - protein, 235 aa, synthetic construct
  REGION 1..235: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSELAKKALEIIERAAEELKKSPDPEAQKEAKKAEQKVREESGSHHWGSGLNDIFEAQKIEWHEGSHHHHHH

SEQ ID NO: 163 - protein, 204 aa, synthetic construct
  REGION 1..204: note = Synthetic
TTNFHLINGSEEARQLIEKAVRAISKKEGTEVHFEKSDGTLEIRVKNLHEKREREIKKVIELILEVAAAERIVRERPGSNLAKKALEIILRAAEELAKADVDAALEAAVRAAEKVVREQPGSNLAKKALEIILRAAEELAKLPDPEALKEAVKAAEKVVREQPGSELAKKALEIIERAAEELKKSPDPEAQKEAKKAEQKVREE

SEQ ID NO: 164 - protein, 235 aa, synthetic construct
  REGION 1..235: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSELAKKALEIIERAAEELKKSPDPEAQKEAKKAEQKVREESGSHHWGSGLNDIFEAQKIEWHEGSHHHHHH

SEQ ID NO: 165 - protein, 204 aa, synthetic construct
  REGION 1..204: note = Synthetic
NTHFIVVHGSEDAAQLAEELVREISKKEGTEVRFEKKDGLLSIEVKNLSEERQREIQKALQLVQDVANAERVVRERPGSNLAKKALEIILRAAEELAKLDLKASLKAAVRAAEKVVREQPGSNLAKKALEIILRAAEELAKLPDPEALKEAVKAAEKVVREQPGSELAKKALEIIERAAEELKKSPDPEAQKEAKKAEQKVREE

SEQ ID NO: 166 - protein, 293 aa, synthetic construct
  REGION 1..293: note = Synthetic
HHHHHHGSGLNDIFEAQKIEWHEAENLYFQSGSDEEVQEAVERAEELREEAEELIKKARKTGDAELLRKALEALEEAVRAVEEAIKRNPDNDEAVETAVRLARELKKVAEELQERAKKTGDAELLKLALRALEVAVRAVELAIKSNPDNDEAVETAVRLAKELLKVAILLAKRAQETGDKELEKLARRALEVAKRAVELAIKSNPDNKEARILKLLLELAELLIELALRGTIIIVEVHINGERQTKYLILAPVEELLKHLERIEEKIKREGASEVEVKVTSGGTTWTFNIKGS

SEQ ID NO: 167 - protein, 258 aa, synthetic construct
  REGION 1..258: note = Synthetic
DEEVQEAVERAEELREEAEELIKKARKTGDAELLRKALEALEEAVRAVEEAIKRNPDNDEAVETAVRLARELKKVAEELQERAKKTGDAELLKLALRALEVAVRAVELAIKSNPDNDEAVETAVRLAKELLKVAILLAKRAQETGDKELEKLARRALEVAKRAVELAIKSNPDNKEARILKLLLELAELLIELALRGTIIIVEVHINGERQTKYLILAPVEELLKHLERIEEKIKREGASEVEVKVTSGGTTWTFNIK

SEQ ID NO: 168 - protein, 217 aa, synthetic construct
  REGION 1..217: note = Synthetic
TWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKALEIILRAAEELAKLPDPEALKEAVKAAEKVVREQPGSELAKKALEIIERAAEELKKSPDPEAQKEAKKAEQKVREERPGSSGSHHHHHH

SEQ ID NO: 169 - protein, 208 aa, synthetic construct
  REGION 1..208: note = Synthetic
TWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 170 - protein, 217 aa, synthetic construct
  REGION 1..217: note = Synthetic
SSIFLLSNVDESARQLAEELVREISKKEGTEVRFEKDDGFLTIEVKNLSEERLREIARALQLIVDVANAERVVRERPGSNLAKKALEIILRAAEELAKLPLKASLKAAVIAAELVVREQPGSNLAKKALEIILRAAEELAKLPDPEALKEAVKAAEKVVREQPGSELAKKALEIIERAAEELKKSPDPEAQKEAKKAEQKVREERPGSSGSHHHHHH

SEQ ID NO: 171 - protein, 208 aa, synthetic construct
  REGION 1..208: note = Synthetic
SSIFLLSNVDESARQLAEELVREISKKEGTEVRFEKDDGFLTIEVKNLSEERLREIARALQLIVDVANAERVVRERPGSNLAKKALEIILRAAEELAKLPLKASLKAAVIAAELVVREQPGSNLAKKALEIILRAAEELAKLPDPEALKEAVKAAEKVVREQPGSELAKKALEIIERAAEELKKSPDPEAQKEAKKAEQKVREERPGS

SEQ ID NO: 172 - protein, 264 aa, synthetic construct
  REGION 1..264: note = Synthetic
GGSDVEWRYTNISEETQQKSAEFVLEIALRAGTGVTFTTRQGELQIQVHNLDELLAIAMLCYTLGLLLGDHRVQELAKRAVEAWERGDEERVKKLLIEALKRLVETAEEVVRERPGSNLAKLALEIILRAAEALARAEDPESLKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGSGGSGSHHWGSGSHHHHHH

SEQ ID NO: 173 - protein, 243 aa, synthetic construct; identical to SEQ ID NO: 14
  REGION 1..243: note = Synthetic

SEQ ID NO: 174 - protein, 241 aa, synthetic construct
  REGION 1..241: note = Synthetic
GGSTVTFDITNIDWKSAELIMLAVYDIAQQEGTDVTFSFKEGELQITVKNLHEKWKRLIEMLIEACRRAQDPDPESLKEAVRIAEELVRLHPGNPLARAALKVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGSGGSGSHHWGSGSHHHHHH

SEQ ID NO: 175 - protein, 220 aa, synthetic construct; identical to SEQ ID NO: 16
  REGION 1..220: note = Synthetic

SEQ ID NO: 176 - protein, 267 aa, synthetic construct
  REGION 1..267: note = Synthetic
GGSPHQFYVYQIDEHVAQLIEKFVRDISRREGTEVRFEKRDGQLEIEVKNLHEAQAIAIGIYIMILLLHQSGTSEDEIAEEIAKLIKGFIEHLKREGSSYEVICEAVAAAVAAIVKALKGCGTSEDEIAEIVARVISEVIRTLKESGSSYEVICECVARIVAEIVQALKESGTSEDEIAEIVARVISEVIRTLKESGSSYEVIKECVQRIVEEIVEALKRSGTSEDEINEIVRRVKSEVERTLKESGSSGGSGSHHWGSGSHHHHHH

SEQ ID NO: 177 - protein, 246 aa, synthetic construct; identical to SEQ ID NO: 18
  REGION 1..246: note = Synthetic

SEQ ID NO: 178 - protein, 241 aa, synthetic construct
  REGION 1..241: note = Synthetic
GGSTVTFDITNISHEAIEIILYGVLGIAAMEGTEVTFHSERGQLQIEVKNLHEKQKRNIEKLIEAALRAQSPDPEDLKEAVRIAEELVRAHPGTPLAHAALQVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGSGGSGSHHWGSGSHHHHHH

SEQ ID NO: 179 - protein, 220 aa, synthetic construct; identical to SEQ ID NO: 20
  REGION 1..220: note = Synthetic

SEQ ID NO: 180 - protein, 206 aa, synthetic construct
  REGION 1..206: note = Synthetic
GGSSHSFILGQASEEARQEIEEVVEAISRKLGTEVRFEKKDGTLHIEVKNLHDEYAQLIADAILLIILAQESDDSEAKKVARLALEIVAQLPNTELAHEALKLAEEALKSTDSEALKVVYLALRIVQQLPDTELAREALELAKEAVKSTDQEALKSVYEALQRVQDKPNTEEARESLERAKEDVKSTDGGSGSHHWGSGSHHHHHH

SEQ ID NO: 181 - protein, 185 aa, synthetic construct; identical to SEQ ID NO: 24
  REGION 1..185: note = Synthetic

SEQ ID NO: 182 - protein, 258 aa, synthetic construct
  REGION 1..258: note = Synthetic
GGSDVEWRFTNVSEEEQEKLARFVLQVAQLAGTQVIFTTRPGELRIRVHNLDELLALAIELYAQGLRLGDKHVQHLAKKAIEAILRGDRKLARFLLEAARAMSRATERPGSNLAKKALEEILRLAEELAKDPDPESLKAAVHCAEFVVRYQPGSNLAKKALEIILRAAEELAKLPDPEALKEAVKAAEKVVREQPGSELAKKALEIIERAAEELKKSPDPEAQKEAKKAEQKVREERPGSGGSGSHHWGSGSHHHHHH

SEQ ID NO: 183 - protein, 237 aa, synthetic construct; identical to SEQ ID NO: 26
  REGION 1..237: note = Synthetic

SEQ ID NO: 184 - protein, 241 aa, synthetic construct
  REGION 1..241: note = Synthetic
GGSTVTFDITNIDDKSTKLIATAVIHIAGREGTTVHFQGHDGQLEIEVKNLHEKWKRLIEMLIEACRRAQDPDPESLKEAVRIAEELVRLHPGNMLAEAALKVILTAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGSGGSGSHHWGSGSHHHHHH

SEQ ID NO: 185 - protein, 220 aa, synthetic construct; identical to SEQ ID NO: 28
  REGION 1..220: note = Synthetic

SEQ ID NO: 186 - protein, 582 aa, synthetic construct
  REGION 1..582: note = Synthetic
HHHHHHGSGSNTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGNVEVAVKAARVAVEAAKQAGDNDVLRKVAEQALRIAKEALKQGNFRVAIEALKVAEEAAKQAGDQDVLKKVEELKLEIFAKAIEDLVRKMGVEMLVFKAGRAVIVVIRGLHPEQAKQLLRFVSQLAHDLGVTVTLTFHGDVVFILVLVGASEEEQKVMQLAIQLLARIIHEAKRRGVSEEALKAIAEFVAIVLEALKRAGILSEEALELATRLLKEVLENAQREGYDESEAIRAAAEALKRVAEAAKRAGITSSEVLELAIRLIKEVVENAQREGYDISEAARAAAEAFKRVAEAAKRAGITSSEVLELAIILIKLVVELAQRKGYDISEAARAAAELFKRLAEALKRAGKTSERALALLILLLAIEILVRDMGVTMETHPSGNEVKVVIKGLHIKQQRQLYRLVREAAKLLGVEVEIEVEGDTVTIVVRGGS

SEQ ID NO: 187 - protein, 570 aa, synthetic construct
  REGION 1..570: note = Synthetic
NTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGNVEVAVKAARVAVEAAKQAGDNDVLRKVAEQALRIAKEALKQGNFRVAIEALKVAEEAAKQAGDQDVLKKVEELKLEIFAKAIEDLVRKMGVEMLVFKAGRAVIVVIRGLHPEQAKQLLRFVSQLAHDLGVTVTLTFHGDVVFILVLVGASEEEQKVMQLAIQLLARIIHEAKRRGVSEEALKAIAEFVAIVLEALKRAGILSEEALELATRLLKEVLENAQREGYDESEAIRAAAEALKRVAEAAKRAGITSSEVLELAIRLIKEVVENAQREGYDISEAARAAAEAFKRVAEAAKRAGITSSEVLELAIILIKLVVELAQRKGYDISEAARAAAELFKRLAEALKRAGKTSERALALLILLLAIEILVRDMGVTMETHPSGNEVKVVIKGLHIKQQRQLYRLVREAAKLLGVEVEIEVEGDTVTIVVRG

SEQ ID NO: 188 - protein, 624 aa, synthetic construct
  REGION 1..624: note = Synthetic
HHHHHHGSGSNTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGNVEVAVKAARVAVEAAKQAGDNDVLRKVAEQALRIAKEAEKQGNVEVAIKALEVAAEAAAQAGDKDVLKKILEQLERLAELAKKQGNKELAIKIFELFIKVIVALMGVRMLSHKGGNAVIVVIEGLHPSQAEQLLRLVHRIAKKAGVTVHLVFTGDIVVIMVVVGASEEEQELMHELVRLIAEALHEAKRLGANEEFLEQLLKLLTLVVRAALRTGSDEARQALEELARIAKEALEEGNAELAKFAIRLLEWLARLYSGSDVASLAVKAIAKIAETALRNGNADTAKEAIQRLEDLARDYSGSDVASLAVKAIAKIAETALRNGDADTAKEAIQRLEDLARDYSGSDVASLAVEAILRIALIALANGNEETAEEARKRLRELAEDYKGSEVAKLAESAERLIEILKIIAKTVRKMGVTMDVRPSGTEVEVVIKGLHIKQQRQLYRDVREAAKKLGVEVEIEVEGDTVTIVVRGGS

SEQ ID NO: 189 - protein, 612 aa, synthetic construct
  REGION 1..612: note = Synthetic
NTHFIVVHGTEEARQLAEEIVRLIAKALGTEVRFEKKDGLLSIEVKNLPEEAQRLIQKLLQLAVRIAAAAKSGDNDVLRKLAEDALRLAKEAEKLGDLGAAAVAARLAVEAAKQAGDNDVLRKVAEQALRIAKEAEKQGNVEVAVKAARVAVEAAKQAGDNDVLRKVAEQALRIAKEAEKQGNVEVAIKALEVAAEAAAQAGDKDVLKKILEQLERLAELAKKQGNKELAIKIFELFIKVIVALMGVRMLSHKGGNAVIVVIEGLHPSQAEQLLRLVHRIAKKAGVTVHLVFTGDIVVIMVVVGASEEEQELMHELVRLIAEALHEAKRLGANEEFLEQLLKLLTLVVRAALRTGSDEARQALEELARIAKEALEEGNAELAKFAIRLLEWLARLYSGSDVASLAVKAIAKIAETALRNGNADTAKEAIQRLEDLARDYSGSDVASLAVKAIAKIAETALRNGDADTAKEAIQRLEDLARDYSGSDVASLAVEAILRIALIALANGNEETAEEARKRLRELAEDYKGSEVAKLAESAERLIEILKIIAKTVRKMGVTMDVRPSGTEVEVVIKGLHIKQQRQLYRDVREAAKKLGVEVEIEVEGDTVTIVVRG

SEQ ID NO: 190 - protein, 75 aa, synthetic construct
  REGION 1..75: note = Synthetic
  REGION 1..45: note = MISC_FEATURE - X is any amino acid
  REGION 47..48: note = MISC_FEATURE - X is any amino acid
  REGION 51..52: note = MISC_FEATURE - X is any amino acid
  REGION 66..69: note = MISC_FEATURE - X is any amino acid
  SITE 73: note = MISC_FEATURE - X is any amino acid
XXXXXXXXXXXXXXXXXXXXXXXXXXXXKXXXXXXXXXXXXXXXXRXXVHXXLRKQGVVAVTQKHXXXXTIYXTE

SEQ ID NO: 191 - protein, 65 aa, synthetic construct
  REGION 1..65: note = Synthetic
  REGION 10..41: note = MISC_FEATURE - X is any amino acid
  REGION 43..47: note = MISC_FEATURE - X is any amino acid
  SITE 49: note = MISC_FEATURE - X is any amino acid
  REGION 51..52: note = MISC_FEATURE - X is any amino acid
  SITE 55: note = MISC_FEATURE - X is any amino acid
  SITE 59: note = MISC_FEATURE - X is any amino acid
  REGION 62..63: note = MISC_FEATURE - X is any amino acid
SVEFHIVNIXXXXXXXXXXXXXXXXXXXXXXXXXXXXXXEXTXXXXXLXEXXLQXILEXIEXXNK

SEQ ID NO: 192 - DNA, 21 bp, synthetic construct
  misc_feature 1..21: note = Synthetic
  source 1..21: mol_type = other DNA
taaagaaggagatatcatatg

SEQ ID NO: 193 - protein, 8 aa, synthetic construct
  REGION 1..8: note = Synthetic
GGSHHWGS

SEQ ID NO: 194 - protein, 6 aa, synthetic construct
  REGION 1..6: note = Synthetic
HHHHHH

SEQ ID NO: 195 - protein, 15 aa, synthetic construct
  REGION 1..15: note = Synthetic
GLNDIFEAQKIEWHE

SEQ ID NO: 196 - protein, 379 aa, synthetic construct
MDVEWRYTNISEETQQKSAEFVLEIALRAGTGVTFTTRQGELQIQVHNLDELLAIAMLCYTLGLLLGDHRVQELAKRAVEAWERGDEERVKKLLIEALKRLVETAEEVVRERPGSNLAKLALEIILRAAEALARAEDPESLKEAVKAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRGGSGSGSSRGPYPYDVPDYA

SEQ ID NO: 197 - protein, 359 aa, synthetic construct
DVEWRYTNISEETQQKSAEFVLEIALRAGTGVTFTTRQGELQIQVHNLDELLAIAMLCYTLGLLLGDHRVQELAKRAVEAWERGDEERVKKLLIEALKRLVETAEEVVRERPGSNLAKLALEIILRAAEALARAEDPESLKEAVKAAEKVVREQPGSNLAKKALEIILRAAEELAKLPDPEALKEAVKAAEKVVREQPGSNLAKKALEIILRAAEELAKLPDPEALKEAVKAAEKVVREQPGSNLAKKALEIILRAAAALANLPDPESRKEADKAADKVRREQPGSELAVVAAIISAVARMGVKMELHPSGNEVKVVIKGLHIKQQRQLYRDVREAAKKAGVEVEIEVEGDTVTIVVRG

SEQ ID NO: 198 - protein, 378 aa, synthetic construct
CVEELLLLARAAHHSGTTVEEAYKLAKKLGISVKELLLLARAAHNSGTTVEEAYKLALKLGISVEELLLLAKAAHYSGTTVEEAYKLALELGISVRELLLLAKAAHFAGRTVREAYALCLALGALRLEDRARELIKEAEKKGDPEKLREALEALEEAVRLVEEAIKLRPDMDLAVEIAVRLARMLKRVAELLQELAKKTGDPELLKLALRALEVAVRAVELAIKSNPDNDEAVETAVRLARELAKVAEELIERAKKTGDKELLKLAKRALEVAMRAVSLALKSNPDNEEARRVAAELVLLVIRAAVIEMGVTMEEHRSGNRVKVVIKGLHESQQEQLLEDVLRAAEIAGVRVRIRFKGDTVTIVVEGSGSGSHHHHHH

SEQ ID NO: 199 - protein, 367 aa, synthetic construct
CVEELLLLARAAHHSGTTVEEAYKLAKKLGISVKELLLLARAAHNSGTTVEEAYKLALKLGISVEELLLLAKAAHYSGTTVEEAYKLALELGISVRELLLLAKAAHFAGRTVREAYALCLALGALRLEDRARELIKEAEKKGDPEKLREALEALEEAVRLVEEAIKLRPDMDLAVEIAVRLARMLKRVAELLQELAKKTGDPELLKLALRALEVAVRAVELAIKSNPDNDEAVETAVRLARELAKVAEELIERAKKTGDKELLKLAKRALEVAMRAVSLALKSNPDNEEARRVAAELVLLVIRAAVIEMGVTMEEHRSGNRVKVVIKGLHESQQEQLLEDVLRAAEIAGVRVRIRFKGDTVTIVVEG